(19) United States
(12) Patent Application Publication
(10) Pub. No.: US 2012/0021995 A1
(43) Pub. Date: Jan. 26, 2012

(54) Polypeptides that Bind TRAIL-R1 and TRAIL-R2

(75) Inventors: Katherine Bowdish, Del Mar, CA (US); Anke Kretz-Rommel, San Diego, CA (US); Mark Renshaw, San Diego, CA (US); Bing Lin, San Diego, CA (US); Jean de Silva Correia, San Diego, CA (US); Roger Ferrini, San Diego, CA (US); Elise Chen, Del Mar, CA (US)

(73) Assignee: ANAPHORE, INC., La Jolla, CA (US)

(21) Appl. No.: 13/190,914

(22) Filed: Jul. 26, 2011

Related U.S. Application Data
(63) Continuation-in-part of application No. 12/577,067, filed on Oct. 9, 2009.
(60) Provisional application No. 61/367,864, filed on Jul. 26, 2010.
(60) Provisional application No. 61/104,358, filed on Oct. 10, 2008.

(51) Int. Cl. A61K 38/16; A61P 35/00; A61K 38/10; C07K 7/04; C07K 14/00

(52) U.S. Cl. 514/19.3; 530/326; 530/324

(57) ABSTRACT

Agonists for TRAIL death receptors including polypeptides that bind to TRAIL death receptor TRAIL-R1 (DR4) and/or TRAIL-R2 (DR5) and optionally having a multimerizing, e.g. trimerizing domain. Agonists are described that do not bind to TRAIL decoy receptors. The multimerizing domain may be derived from human tetranectin. The agonists can induce apoptosis in pathogenic cells expressing a TRAIL death receptor. Pharmaceutical compositions are described for treating diseases associated with cells expressing DR4 and DR5, such as tumor cells. Methods for selecting polypeptides and preparing multimeric complexes.

### CROSS-REFERENCE TO RELATED APPLICATION

This application claims the benefit of U.S. provisional application 61/367,684 filed Jul. 26, 2010 and is a continuation in part of U.S. application Ser. No. 12/577,067, filed Oct. 9, 2009, which claims the benefit of U.S. Provisional Application Ser. No. 61/104,358, filed Oct. 10, 2008, each of which is incorporated by reference herein in their entirety.

### SEQUENCE LISTING STATEMENT

The sequence listing is filed in this application in electronic format only and is incorporated by reference herein. The sequence listing text file “08-831-US-CIP_SEQLIST.txt” was created on Jul. 26, 2010, and is 413,414 bytes in size.

### FIELD OF THE INVENTION

The invention relates broadly to the treatment of cancer and other disorders. In particular, the invention relates to polypeptides that bind to a TRAIL death receptor and that induce apoptosis in pathogenic cells expressing a TRAIL death receptor.

### BACKGROUND OF THE INVENTION

TRAIL (tumor necrosis factor-related apoptosis-inducing ligand, also referred to in the literature as Apo2L and TNFSF10, among other things) belongs to the tumor necrosis factor (TNF) superfamily and has been identified as an activator of programmed cell death, or apoptosis, in tumor cells. TRAIL is expressed in cells of the immune system including NK cells, T cells, macrophages, and dendritic cells and is located in the cell membrane. TRAIL can be processed by cysteine proteases, generating a soluble form of the protein. Both the membrane-bound and soluble forms of TRAIL function as trimers and are able to trigger apoptosis via interaction with TRAIL receptors located on target cells. In humans, five receptors have been identified to have binding activity for TRAIL. Two of these five receptors, TRAIL-R1 (DR4, TNFRSF10a) and TRAIL-R2 (DR5, TNFRSF10b), contain a cytoplasmic region called the death domain (DD). The death domain on these two receptor molecules is required for TRAIL-activation of the extrinsic apoptotic pathway upon the binding of TRAIL to the receptors. The remaining three TRAIL receptors (called TRAIL-R3 (DcR1, TNFRSF10c), TRAIL-R4 (DcR2, TNFRSF10d) and circulating osteoprotegerin (OPG, TNFRSF11b)) are thought to serve as decoy receptors. These three receptors lack functional DDs and are thought to be mainly involved in negatively regulating apoptosis by sequestering TRAIL or stimulating pro-survival signals.

Upon binding of TRAIL to TRAIL-R1 (DR4) or -R2 (DR5) the trimerized receptors recruit several cytosolic proteins that form the death-inducing signaling complex (DISC) which subsequently leads to activation of caspase-8 or caspase-10. This triggers one of two different routes that cause irreversible cell death, one in which caspase-8 directly activates the effector caspases (caspases-3, -6, -7) leading to the disassembly of the cell, and the other route involving the caspase-8 dependent cleavage of the pro-death Bcl-2 family protein, Bid, and engaging the mitochondrial or intrinsic death pathway.

In light of this cell death activity, several TRAIL-based therapeutic approaches are being pursued. In some preclinical studies recombinant soluble TRAIL has induced apoptosis in a broad spectrum of human tumor cell lines derived from leukemia, multiple myeloma, and neuroblastoma, as well as lung, colon, breast, prostate, pancreas, kidney and thyroid carcinoma. Dose-dependent suppression of tumor growth has been observed in multiple tumor xenografts with no or little systemic toxicity (Ashkenazi 1999, Jin 2004). In these studies, the recombinant TRAIL formulation appears to be important for selectivity and antitumor properties, as highly aggregated forms of TRAIL were associated with hepatotoxicity. Recombinant TRAIL has safely been administered to patients.

Several DR4 or DR5 human agonistic monoclonal antibodies are being developed. In cell lines and mouse models, these antibodies potently induced apoptosis. At least five monoclonal antibodies are currently in clinical development either as single agent therapies or combined with small molecule chemotherapeutics. In at least one study, monoclonal anti-DR4 or -DR5 antibodies were overall safe and well tolerated, resulting in a number of patients with stable disease (i.e. they lack sufficient potency on their own), with studies of combination chemotherapy currently being evaluated. Preclinical studies with monoclonal antibodies that bind to DR5 indicate that super-clustering of TRAIL receptors mediated through secondary cross-linking in vitro with a secondary antibody (and in vivo likely through the antibody Fc domain binding to immune cell surface receptors at the tumor site) appears to enhance activity.

Nevertheless, the therapeutic approaches detailed above have several deficiencies. For example, while native/recombinant TRAIL can bind both DR4 and DR5 (both of the DD containing receptors), it also binds to the decoy receptors, broadly limiting its activity. Additionally TRAIL has a very short half-life, on the order of minutes, which further limits its potency. Each antibody approach, while providing molecules with longer half-lives, is specific for a single given receptor. Furthermore, the large size of antibodies can limit their tumor penetration.

Accordingly, there is a need in the art for additional molecules that bind to DR4 and DR5, compositions comprising those molecules, methods for screening for such molecules, and methods for using such molecules in the therapeutic treatment of a wide variety of cancers.

### SUMMARY OF THE INVENTION

In one aspect, the invention is directed to a TRAIL death receptor agonist including a polypeptide that binds to TRAIL death receptor DR4 and includes a C-Type Lectin Like Domain (CLTD) having one of the following combinations of sequences in loops 1 and 4:

| | Loop 1 | | Loop 4 SEQ |
|---|---|---|---|
| Loop 1 | SEQ ID NO | Loop 4 | ID NO |
| GWLEGSGW | 428 | DGGVQWRWEN | 436 |
| GYMTGVGW | 429 | DGGRSWKWEN | 437 |
| GWMEGVGW | 430 | DGGPPWRWEN | 438 |
| GWLEGSGW | 428 | DGGFPARWEN | 439 |
| GWMDGSGW | 431 | DGGRLWRWEN | 440 |
| GWMAGVGW | 290 | DGGPGLRWEN | 441 |
| GYLAGTGW | 432 | DGGRVLAWEN | 443 |
| GWLAGSGW | 433 | DGGGGWPWEN | 443 |
| GWVAGVGW | 434 | DGGGGWRWEN | 444 |
| GWIEGAGW | 435 | DGGWRSRWEN | 445 |
| GWLEGYGW | 265 | DGGAERAWEN | 446 |
| GWLEGVGW | 261 | DGGWPFSNEN | 315 |

The agonist may include one of the following sequences for loop 3:

| Loop3 SEQ | SEQ ID NO |
|---|---|
| NWGDQRLAQ | 496 |
| NWADERRNQ | 497 |
| NWADKRWLQ | 498 |
| NWKDDRFNQ | 499 |
| NWLDPRMGQ | 500 |
| NWYSDYLNQ | 501 |
| NWHYqKYIQ | 502 |
| NWALDRYNQ | 503 |
| NWGRPELAQ | 504 |
| NWANPSFMQ | 505 |
| NWADERFLQ | 506 |
| NWGRELAQ | 507 |
| NWTQRHSGQ | 451 |
| NWARHINEQ | 452 |
| NWYSWPKLQ | 453 |
| NWGWSARVQ | 457 |
| NWGWMDSKQ | 458 |
| NWWFPTLSQ | 459 |
| NWGDPRWSQ | 545 |
| NWADPKWSQ | 569 |
| NWFHDRFNQ | 570 |

In various embodiments of the invention, Loop 1 of the agonist is SEQ ID NO: 428 and Loop 4 of the agonist is SEQ ID NO: 436.

Still further, the invention is directed to an agonist for a TRAIL death receptor the agonist does not bind to a TRAIL decoy receptor, for example at least one of DcR1, DcR2, and circulating osteoprotegerin (OPG).

Optionally, the agonist polypeptide binds to DR5, and may include a second polypeptide that binds to DR4.

In another embodiment, the invention is directe to a non-natural polypeptide having a trimerizing domain and at agonist for a TRAIL death receptor, wherein the trimerizing domain comprises a polypeptide of SEQ ID NO: 10 having up to five amino acid substitutions at positions 10, 17, 20, 21, 24, 25, 26, 28, 29, 30, 31, 32, 33, 34, or 35, and wherein three trimerizing domains form a trimeric complex. The trimerizing domain may include a trimerizing polypeptide that is derived from a polypeptide selected from the group consisting of hTRAF3 [SEQ ID NO: 2], hMBP [SEQ ID NO: 3], hSPC300 [SEQ ID NO: 4], hNEMO [SEQ ID NO: 5], hcubilin [SEQ ID NO: 6], hThrombospondins [SEQ ID NO: 7], and neck region of human SP-D, [SEQ ID NO: 8], neck region of bovine SP-D [SEQ ID NO: 9], neck region of rat SP-D [SEQ ID NO: 11], neck region of bovine conglutinin: [SEQ ID NO: 12]; neck region of bovine collectin: [SEQ ID NO: 13]; and neck region of human SP-D: [SEQ ID NO: 14]. In another embodiment, the polypeptide is at least 85% identical to SEQ ID NOS: 2-9 and 11-14.

In further aspects of the invention, the agonist includes a polypeptide that binds DR4 positioned at one of the N-terminus and the C-terminus of the trimerizing domain, and a polypeptide sequence that binds a tumor-associated antigen (TAA) or tumor-specific antigen (TSA) at the other of the N-terminus and the C-terminus. In one embodiment, the polypeptide binds to a tumor-associated antigen (TAA) or tumor-specific antigen (TSA) with at least two times greater affinity than the polypeptide binds to DR4 or DR5.

Even further, the agonist includes a polypeptide that binds DR4 positioned at one of the N-terminus and the C-terminus of the trimerizing domain, and a polypeptide sequence that binds a receptor selected from the group consisting of Fn14, FAS receptor, TNF receptor, and LIGHT receptor, at the other of the N-terminus and the C-terminus.

Further aspects of the invention include a trimeric complex of three agonists for a TRAIL death receptor. The trimeric complex may also include three polypeptide sequences that specifically bind DR5, wherein the sequences can be the same or different.

The trimeric complex may be used in a method of inducing apoptosis in a tumor cell in a patient expressing at least one of DR4 and DR5. The method includes contacting the cell with the trimeric complex. In a particular aspect, the trimeric complex induces caspase-dependent apoptosis. The trimeric complexes may be used in pharmaceutical compositions that include the complexes and at least one pharmaceutically acceptable excipient. The pharmaceutical compositions may be used in a method for treating a cancer patient.

### DESCRIPTION OF THE FIGURES

FIG. 1 depicts an alignment of the nucleotide and amino acid sequences of the coding regions of the mature forms of human (SEQ ID NOS: 99 [nucleotide sequence] and 100 [amino acid sequence]) and murine tetranectin (SEQ ID NOS: 15 [nucleotide sequence] and 16 [amino acid sequence]) with an indication of known secondary structural elements.

FIG. 2 shows alignment of the amino acid sequences of the trimerising structural element of members of the tetranectin protein family. Amino acid sequences (one letter code) corresponding to residue E1 to K52 comprising exon 2 and the first three residues of exon 3 of human tetranectin (SEQ ID NO: 1) are shown: murine tetranectin (SEQ ID NO: 17) (Sorensen et al., Gene, 152: 243-245, 1995); tetranectin homologous protein isolated from reefshark cartilage (SEQ ID NO: 24) (Neame and Boynton, 1992, 1996); and tetranectin homologous protein isolated from bovine cartilage (SEQ ID NO: 23) (Neame and Boynton, database accession number PATCHX:u22298). Residues at a and d positions in the heptad repeats are listed in boldface. The listed truncated consensus sequence (SEQ ID NO: 10) of the tetranectin protein family trimerising structural element includes the residues present at a and d positions in the heptad repeats shown in the figure in addition to the other conserved residues of the region (“*” denotes an aliphatic hydrophobic residue).

FIG. 3A, B, C and D show examples of tetranectin trimerizing module truncations for use with exemplary polypeptides of the invention.

FIG. 4 shows an alignment of the amino acid sequences of ten CTLDs of known 3D-structure. The sequence locations of main secondary structure elements are indicated above each sequence, labeled in sequential numerical order as “αN”, denoting a α-helix number N, and “βM”, denoting β-strand number M. The four cysteine residues involved in the formation of the two conserved disulfide bridges of CTLDs are indicated and enumerated in the Figure as “CI”, “CII”, “CIII” and “CIV” respectively. The two conserved disulfide bridges are CI-CIV and CII-CIII, respectively. The various loops 1-4 of loop segment A (LSA) and loop segment B (LSB) (loop 5) in the human tetranectin sequence are indicated by underlining. The ten C-type lectins are hTN: human tetranectin (SEQ ID NO: 117), MBP: mannose binding protein (SEQ ID NO: 118); SP-D: surfactant protein D (SEQ ID NO: 119); LY49A: NK receptor LY49A (SEQ ID NO: 120); H1-ASR: H1 subunit of the asialoglycoprotein receptor (SEQ ID NO: 121); MMR-4:macrophage mannose receptor domain 4 (SEQ ID NO: 122); IX-A (SEQ ID NO: 123) and IX-B (SEQ ID NO: 124): coagulation factors IX/X-binding protein domain A and B, respectively; Lit: lithostatine (SEQ ID NO: 125); TU14: tunicate C-type lectin (SEQ ID NO: 126). All of these CTLDs are from human proteins except TU14.

FIG. 5 depicts an alignment of several C-type lectin domains from tetranectins isolated from human (Swissprot P05452) (SEQ ID NO: 127), mouse (Swissprot P43025) (SEQ ID NO: 128), chicken (Swissprot Q9DDD4) (SEQ ID NO: 129), bovine (Swissprot Q2KIS7) (SEQ ID NO: 130), Atlantic salmon (Swissprot B5XCV4) (SEQ ID NO: 131), frog (Swissprot Q510R9) (SEQ ID NO: 132), zebrafish (GenBank XP 701303) (SEQ ID NO: 133), and related CTLD homologues isolated from cartilage of cattle (Swissprot u22298) (SEQ ID NO: 134) and reef shark (Swissprot p26258) (SEQ ID NO: 135).

FIG. 6 shows the PCR strategy for creating randomized loops in a CTLD.

FIG. 7 shows the DNA and amino acid sequence of the human tetranectin CTLD modified to contain restriction sites for cloning, indicating the Ca2+ binding sites. Restriction sites are underscored with solid lines. Loops are underlined with dashed lines. Calcium coordinating residues are in bold italics and include Site 1: D116, E120, G147, E150, N151; Site 2: Q143, D145, E150, D165. The CTLD domain starts at amino acid A45 in bold (i.e. ALQTVCL . . . ). Changes to the native tetranectin (TNCTLD) base sequence are shown in lower case. The restriction sites were created using silent mutations that did not alter the native amino acid sequence.

FIG. 8 depicts results of experiments showing ED50 values for clones generated to bind human DR4.

FIG. 9 depicts results of experiments showing agonist activity of hybrid clones on ST486 cancer cells expressing DR4.

FIGS. 10A-10D depicts results of experiments showing agonist activity of hybrid clones on additional cancer cells expressing DR4.

FIG. 11 depicts results of experiments showing agonist cell killing activity is not present on cancer cells lacking DR4 expression.

FIGS. 12(A) and 12(B) depicts results of experiments showing DR4 specific ATRIMERS™ do not induce cell death in normal B cells and hepatocytes.

FIG. 13 depicts results of experiments showing agonist activity is mediated through the caspase pathway

FIG. 14 depicts results of experiments showing agonist activity of 71p881B3 affinity matured clones.

FIG. 15 shows a schematic of the peptide phage display library that was constructed to select for peptides which would bind as part of a trimeric conformation when fused to the trimerization domain of human tetranectin.

FIG. 16 depicts results of experiments showing agonist activity of deletion mutations of the clone 132p18P3A10

FIG. 17 depicts results of experiments showing agonist activity of alanine scanning mutations of the clone 132p18P3A10.

FIG. 18 depicts results of experiments showing cell-based killing activity of ATRIMER™ clones in comparison to TRAIL. Activity was measured using ST486, Colo205, and H2122 cells, via binding to DR4.

FIG. 19 depicts results of experiments showing cell-based killing activity of ATRIMER™ clones in comparison to TRAIL. Activity was measured using ST486 cells, via binding to DR4.

FIG. 20 depicts results of experiments showing cell-based killing activity of ATRIMER™ clones in comparison to TRAIL. Activity was measured using Colo205 cells, via binding to both DR4.

FIG. 21 depicts results of experiments showing DR4 specificity of cell-based killing activity of ATRIMER™ clones in comparison to TRAIL. Activity was measured using A2780 cells which express DR5 only.

### DETAILED DESCRIPTION OF THE INVENTION

In various aspects, the invention is directed to TRAIL receptor agonists that include a polypeptide having the backbone structure of a CTLD wherein the polypeptides bind to a TRAIL death receptor. In various embodiments, the agonists also include a multimerizing domain. Two, three, or more of the polypeptides can multimerize to form an agonist that is a multimeric complex including the polypeptides that bind the TRAIL death receptor. Upon binding to a TRAIL death receptor on a cell presenting such receptor, the agonist induces cell apoptosis. In an alternative embodiment, the polypeptide binds the death receptor but is not an agonist for the receptor, allowing targeted delivery of therapeutic agents such as auristatin, maytansinoids, among others, that are associated (e.g., covalently bound to) with the polypeptide. In addition, the invention provides methods for treating cancer and other disorders in a subject by administering an agonist to the subject. The polypeptides include one or more polypeptides that specifically bind to one or both of TRAIL-R1 (DR4) or TRAIL-R2 (DR5), and, preferably, do not bind to a TRAIL decoy receptor.

### DEFINITIONS

Before defining the invention in further detail, a number of terms are defined. Unless a particular definition for a term is provided herein, the terms and phrases used throughout this disclosure should be taken to have the meaning as commonly understood in the art. Also, as used in this specification and the appended claims, the singular forms “a,” “an,” and “the” include plural referents unless the context clearly dictates otherwise.

“TRAIL” or “TRAIL polypeptide” refers to SEQ ID NO: 136, as well as biologically active fragments of SEQ ID NO: 136. Fragments include, but are not limited to, sequences having about 5 to about 50 amino acid residues, or about 5 to about 25, or about 10 to about 20 residues, or about 12 to about 20 amino acid residues of SEQ ID NO: 136. Optionally, the TRAIL peptide consists of no more than 25 amino acid residues (e.g., 25, 23, 21, 19, 17, 15 or less amino acid residues).

The term “TRAIL death receptor” as used herein refers to a protein that binds TRAIL and, upon binding TRAIL, activates programmed cell death (apoptosis) in tumor cells. Certain non-limiting examples of a TRAIL death receptor include either of the receptor proteins commonly referred to as TRAIL-R1 (DR4) (SEQ ID NO: 137) or TRAIL-R2 (DR5) (SEQ ID NO: 138).

The term “DR4,” “DR4 receptor” and “TRAIL-R1” are used interchangeably herein to refer to the full length TRAIL receptor sequence of SEQ ID NO: 137 and soluble, extracellular domain forms of the receptor described in Pan et al., Science, 276:111-113 (1997); WO98/32856 published Jul. 30, 1998; U.S. Pat. No. 6,342,363 issued Jan. 29, 2002; and WO99/37684 published Jul. 29, 1999.

The term “DR5,” “DR5 receptor” and “TRAIL-R2” are used interchangeably herein to refer to the full length TRAIL receptor sequence of SEQ ID NO: 138 and soluble, extracellular domain forms of the receptor described in Sheridan et al., Science, 277:818-821 (1997); Pan et al., Science, 277:815-818 (1997), U.S. Pat. No. 6,072,047 issued Jun. 6, 2000; U.S. Pat. No. 6,342,369, WO98/51793 published Nov. 19, 1998; WO98/41629 published Sep. 24, 1998; Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999, each of which is incorporated herein by reference in its entirety.

The term “TRAIL decoy receptor” as used herein refers to a protein that binds TRAIL and, upon binding TRAIL, does not activate programmed cell death (apoptosis) in tumor cells. Accordingly, TRAIL decoy receptors are believed to function as inhibitors, rather than transducers of programmed cell death signaling. Certain non-limiting examples of a TRAIL decoy receptor include any of the receptor proteins commonly referred to as TRAIL-R3 (also DcR1, TRID, LIT or TNFRSF10c) (SEQ ID NO: 141) [(Pan et al., Science, 276:111-113 (1997) Sheridan et al., Science, 277:818-821 (1997); McFarlane et al., J. Biol. Chem., 272:25417-25420 (1997); Schneider et al., FEBS Letters, 416:329-334 (1997); Degli-Esposti et al., J. Exp. Med., 186:1165-1170 (1997); and Mongkolsapaya et al., J. Immunol., 160:3-6 (1998)] (SEQ ID NO: 139), TRAIL-R4 (also DcR2, TRUNDD and TNFRSF10d) (SEQ ID NO: 140) [Marsters et al., Curr. Biol., 7:1003-1006 (1997); Pan et al., FEBS Letters, 424:41-45 (1998); Degli-Esposti et al., Immunity, 7:813-820 (1997)] and circulating osteoprotegerin (also OPG, TNFRSF11b), each of which is incorporated herein by reference in its entirety.

The term “TRAIL receptor agonist” or “agonist” is used in the broadest sense, and includes any molecule that partially or fully enhances, stimulates or activates one or more biological activities of DR4 or DR5, and biologically active variants thereof, in vitro, in situ, or in vivo. Examples of such biological activities include apoptosis as well as those further reported in the literature. An agonist may function in a direct or indirect manner. For instance, a “TRAIL death receptor agonist” may function to partially or fully enhance, stimulate or activate one or more biological activities of DR4 or DR5, in vitro, in situ, or in vivo as a result of its direct binding to DR4 or DR5, which causes receptor activation or signal transduction. TRAIL receptor agonists include TRAIL polypeptides as defined herein as well as polypeptides that bind to TRAIL receptors that would not be considered a TRAIL polypeptide; for example, polypeptides that specifically bind a TRAIL death receptor but not a TRAIL decoy receptor as identified using the methods described herein.

“ATRIMER™ polypeptide complex,” “ATRIMER™ complex,” or simply “ATRIMER™” refers to a trimeric complex of three trimerizing domains that also include a CLTD (Anaphore, Inc., San Diego, Calif.).

The term “binding member” as used herein refers to a member of a pair of molecules which have binding specificity for one another. The members of a binding pair may be naturally derived or wholly or partially synthetically produced. One member of the pair of molecules has an area on its surface, or a cavity, which binds to and is therefore complementary to a particular spatial and polar organization of the other member of the pair of molecules. Thus the members of the pair have the property of binding specifically to each other.

In various aspects of the invention, the binding members for a TRAIL death receptor are TRAIL receptor agonists. These members include TRAIL polypeptides as described herein, as well as polypeptides including a TRAIL polypeptide and a multimerizing (e.g., trimerizing) domain, and polypeptides including a multimerizing domain and a polypeptide that is not a TRAIL polypeptide, but which binds to and stimulates the TRAIL death receptor, as further described herein. In other aspects, the polypeptides of the invention bind to a TRAIL death receptor but are not agonists for the receptor.

As used herein, the term “multimerizing domain” means an amino acid sequence that comprises the functionality that can associate with two or more other amino acid sequences to form trimers or other multimeric complexes. In one example, the polypeptide contains an amino acid sequence—a “trimerizing domain”—which forms a trimeric complex with two other trimerizing domains. A trimerizing domain can associate with other trimerizing domains of identical amino acid sequence (a homotrimer), or with trimerizing domains of different amino acid sequence (a heterotrimer). Such an interaction may be caused by covalent bonds between the components of the trimerizing domains as well as by hydrogen bond forces, hydrophobic forces, van der Waals forces and salt bridges. In various embodiments so of the invention, the multimerizing domain is a dimerizing domain, a trimerizing domain, a tetramerizing domain, a pentamerizing domain, etc. These domains are capable of forming polypeptide complexes of two, three, four, five or more polypeptides of the invention.

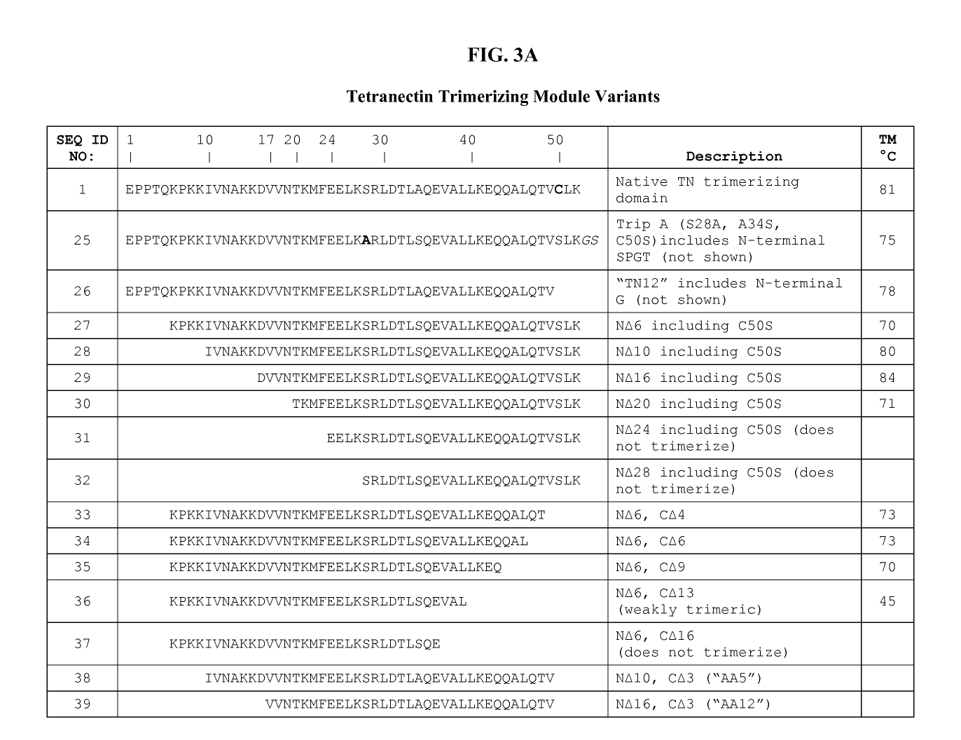

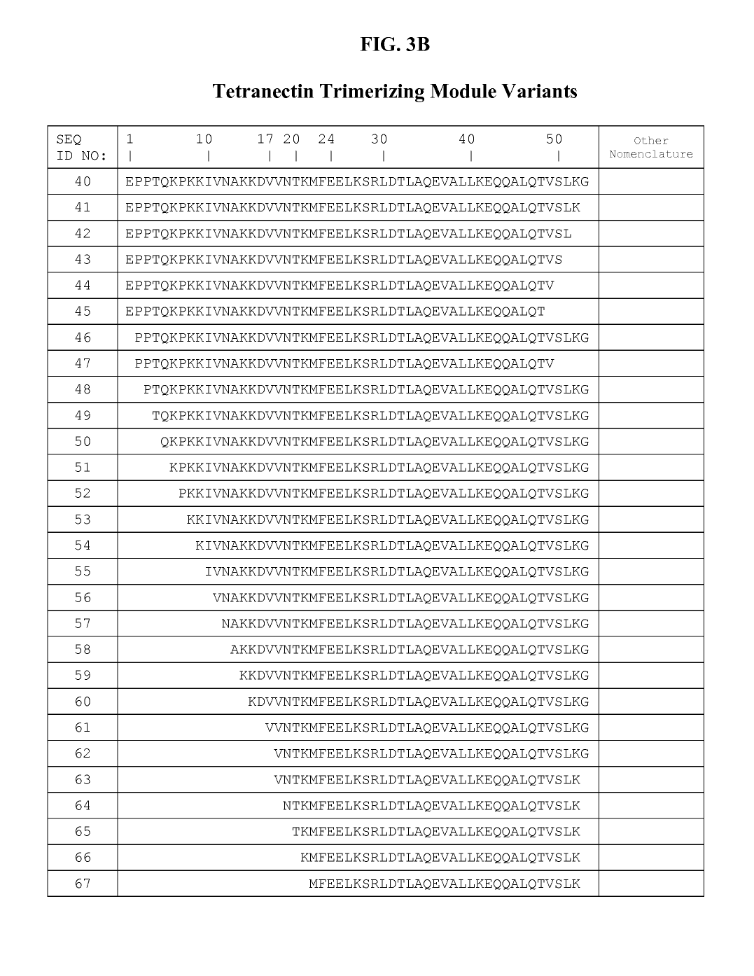

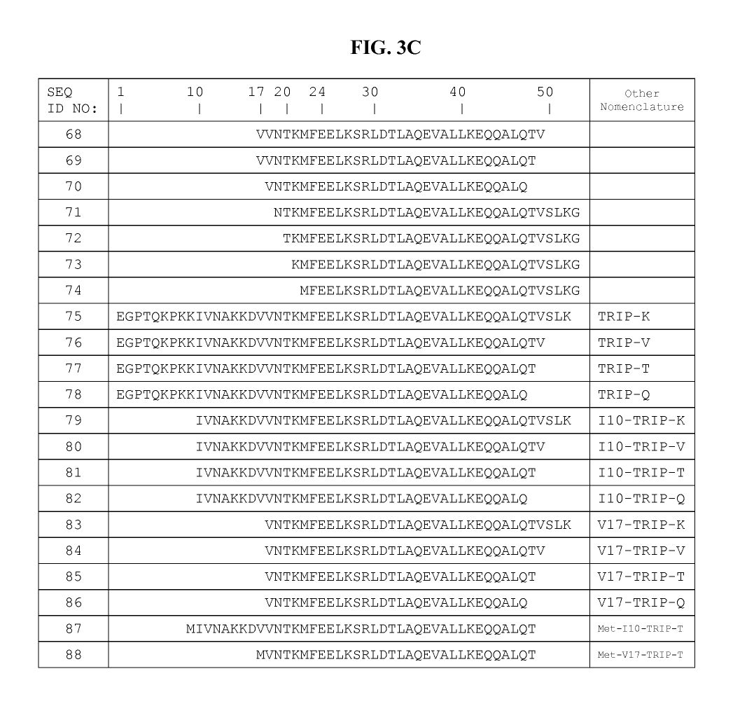

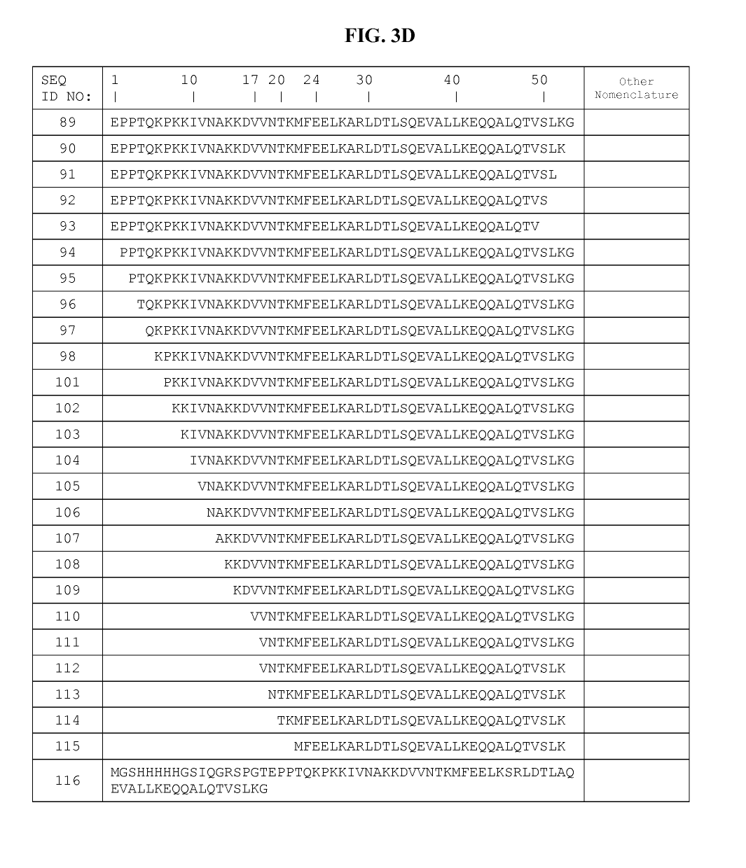

The trimerizing domain of a polypeptide of the invention may be derived from tetranectin as described in U.S. Patent Application Publication No. 2007/0154901 (901 Application), which is incorporated by reference in its entirety. The mature human tetranectin single chain polypeptide sequence is provided herein as SEQ ID NO: 100. Examples of a tetranectin trimerizing domain includes the amino acids 17 to 49, 17 to 50, 17 to 51 and 17-52 of SEQ ID NO: 1, which represent the amino acids encoded by exon 2 of the human tetranectin gene, and optionally the first one, two or three amino acids encoded by exon 3 of the gene. Other examples include amino acids 1 to 49, 1 to 50, 1 to 51 and 1 to 52, which represents all of exons 1 and 2, and optionally the first one, two or three amino acids encoded by exon 3 of the gene. Alternatively, only a part of the amino acid sequence encoded by exon 1 is included in the trimerizing domain. In particular, the N-terminus of the trimerizing domain may begin at any of residues 1, 2, 3, 4, 5, 6, 7, 8, 9, 10, 11, 12, 13, 14, 15, 16 and 17 of SEQ ID NO: 1. In particular embodiments, the N terminus is 110 or V17 and the C-terminus is Q47, T48, V49, C(S)50, L51 or K52 (numbering according to SEQ ID NO: 1). In addition, FIGS. 3A-3D provide a number of potential truncation variants of the human tetranectin trimerizing domain. Furthermore, U.S. Patent Application Publication No 2010/028995 shows a number of human tetranectin trimerizing module truncation variants.

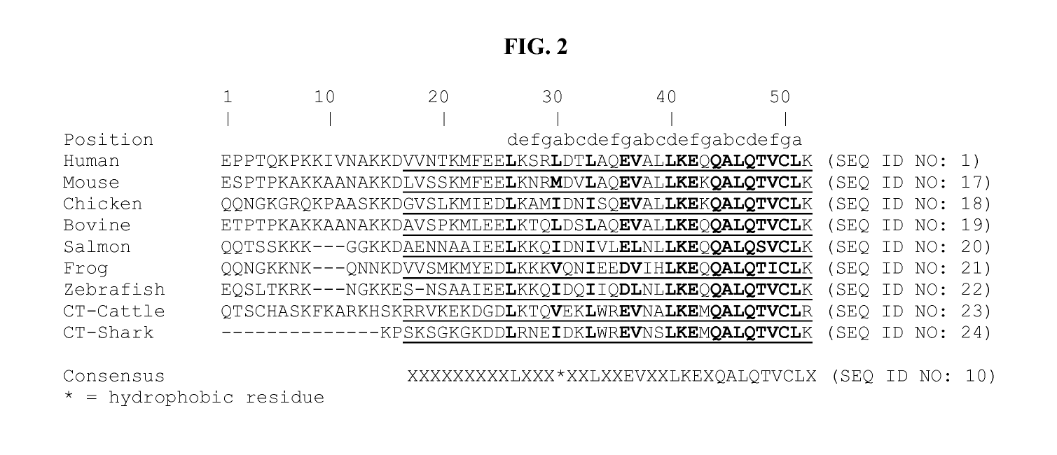

In one aspect of the invention, the trimerizing domain is a tetranectin trimerizing structural element (“TTSE”) having a amino acid sequence of SEQ ID NO: 1 which is a consensus sequence of the tetranectin family trimerizing structural element as more fully described in US 2007/00154901, which is incorporated herein by reference in its entirety. As shown in FIG. 2, the TTSE embraces variants of a naturally occurring member of the tetranectin family of proteins, and in particular variants that have been modified in the amino acid sequence without adversely affecting, to any substantial degree, the ability of the TTSE to form alpha helical coiled coil trimers. In various aspects of the invention, the trimeric polypeptide according to the invention includes a TTSE as a trimerizing domain having at least 66% amino acid sequence identity to the consensus sequence of SEQ ID NO: 10; for example at least 73%, at least 80%, at least 86% or at least 92% sequence identity to the consensus sequence of SEQ ID NO: 10 (counting only the defined (not X) residues). In other words, at least one, at least two, at least three, at least four, or at least five of the defined amino acids in SEQ ID NO: 10 may be substituted.

In one particular embodiment, the cysteine at position 50 (C50) of SEQ ID NO: 100 can be advantageously be mutagenized to serine, threonine, methionine or to any other amino acid residue in order to avoid formation of an unwanted inter-chain disulphide bridge, which can lead to unwanted multimerization. Other known variants include at least one amino acid residue selected from amino acid residue nos. 6, 21, 22, 24, 25, 27, 28, 31, 32, 35, 39, 41, and 42 (numbering according to SEQ ID NO: 1), which may be substituted by any non-helix breaking amino acid residue. These residues have been shown not to be directly involved in the intermolecular interactions that stabilize the trimeric complex between three TTSEs of native tetranectin monomers. In one aspect shown in FIG. 2, the TTSE has a repeated heptad having the formula a-b-c-d-e-f-g (N to C), wherein residues a and d (i.e., positions 26, 30, 33, 37, 40, 44, 47, and 51 may be any hydrophobic amino acid (numbering according to SEQ ID NO: 1).

In further embodiments, the TTSE trimerization domain may be modified by the incorporation of polyhistidine sequence and/or a protease cleavage site, e.g, Blood Coagulating Factor Xa or Granzyme B (see US 2005/0199251, which is incorporated herein by reference), and by including a C-terminal KG or KGS sequence. Also, to assist in purification, Proline at position 2 may be substituted with Glycine.

Particular non-limiting examples of TTSE truncations and variants are shown in FIGS. 3A-3D. In addition, a number of trimerizing domains having substantial homology (greater than 66%) to the trimerizing domain of human tetranectin are known:

TABLE 1

| | | |
|---|---|---|
| Equus caballus TN-like | KMFEELKSQLDSLAQEVALLKEQQALQTVCL | SEQ ID NO: 142 |
| Cat TN | KMFEELKSQVDSLAQEVALLKEQQALQTVCL | SEQ ID NO: 143 |
| Mouse TN | SKMFEELKNRMDVLAQEVALLKEKQALQTVCL | SEQ ID NO: 144 |
| Rat TN | KMFEELKNRLDVLAQEVALLKEKQALQTVCL | SEQ ID NO: 145 |
| Bovine TN | KMLEELKTQLDSLAQEVALLKEQQALQTVCL | SEQ ID NO: 146 |
| Equus caballus CTLD | DLKTQVEKLWREVNALKEMQALQTVCL | SEQ ID NO: 147 |
| like | | |
| Canis lupus CTLD | DLKTQVEKLWREVNALKEMQALQTVCL | SEQ ID NO: 148 |
| member A | | |
| Bovine CTLD member A | DLKTQVEKLWREVNALKEMQALQTVCL | SEQ ID NO: 149 |
| Macaca mulatta CTLD | DLKTQIEKLWTEVNALKEIQALQTVCL | SEQ ID NO: 150 |
| member A | | |
| Taeniopygia guttata | DDLKTQIDKLWREVNALKEIQALQTVCL | SEQ ID NO: 151 |
| CTLD member A | | |
| Ornithorhynchus | DLKTQVEKLWREVNALKEMQALQTVCL | SEQ ID NO: 152 |
| anatinus CTLD like | | |
| Rat CTLD member A | DLKSQVEKLWREVNALKEMQALQTVCL | SEQ ID NO: 153 |
| Monodelphis domestica | DLKTQVEKLWREVNALKEMQALQTVCL | SEQ ID NO: 154 |
| CTLD member A | | |
| Shark TN | DDLRNEIDKLWREVNSLKEMQALQTVCL | SEQ ID NO: 155 |
| Taeniopygia guttata | KMIEDLKAMIDNISQEVALLKEKQALQTVCL | SEQ ID NO: 156 |
| TN-like | | |
| Gallus gallus TN | KMIEDLKAMIDNISQEVALLKEKQALQTVCL | SEQ ID NO: 157 |
| Danio rerio CTLD | DDMKTQIDKLWQEVNSLKEMQALQTVCL | SEQ ID NO: 158 |
| member A | | |
| Gallus gallus, CTLD | DDLKTQIDKLWREVNALKEMQALQSVCL | SEQ ID NO: 159 |
| member A | | |
| Mouse CTLD member A | DDLKSQVEKLWREVNALKEMQALQTVCL | SEQ ID NO: 160 |
| Gallus gallus CTLD | DDLKTQIDKLWREVNALKEMQALQSVCL | SEQ ID NO: 161 |
| member A | | |
| Tetraodon | DDVRSQIEKLWQEVNSLKEMQALQTVCL | SEQ ID NO: 162 |
| nigroviridis, unkown | | |
| Xenopus laevis | DLKTQIDKLWREINSLKEMQALQTVCL | SEQ ID NO: 163 |
| MGC85438 | | |
| Tetraodon | EELRRQVSDLAQELNILKEQQALHTVCL | SEQ ID NO: 164 |
| nigroviridis, unkown | | |
| Xenopus laevis, unkown | KMYEELKQKVQNIELEVIHLKEQQALQTICL | SEQ ID NO: 165 |
| Xenopus tropicalis TN | KMYEDLKKKVQNIEEDVIHLKEQQALQTICL | SEQ ID NO: 166 |
| Salmo salar TN | EELKKQIDNIVLELNLLKEQQALQSVCL | SEQ ID NO: 167 |
| Danio rerio TN | EELKKQIDQIIQDLNLLKEQQALQTVCL | SEQ ID NO: 168 |
| Tetraodon | EQMQKQINDIVQELNLLKEQQALQAVCL | SEQ ID NO: 169 |
| nigroviridis, unknown | | |
| Tetraodon | EQMQKQINDIVQELNLLKEQQALQAVCL | SEQ ID NO: 170 |
| nigroviridis, unkown | | |

Other human polypeptides that are known to trimerizing include:

| | | |
|---|---|---|
| hTRAF3 | NTGLLESQLSRHDQMLSVHDIRLADMD | SEQ ID NO: 2 |
| | LRFQVLETASYNGVLIWKIRDYKRRKQ | |
| | EAVM | |
| hMBP | AASERKALQTEMARIKKWLTF | SEQ ID NO: 3 |
| hSPC300 | FDMSCRSRLATLNEKLTALERRIEYIE | SEQ ID NO: 4 |
| | ARVTKGETLT | |
| hNEMO | ADIYKADFQAERQAREKLAEKKELLQE | SEQ ID NO: 5 |
| | QLEQLQREYSKLKASCQESARI | |
| hcubilin | LTGSAQNIEFRTGSLGKIKLNDEDLSE | SEQ ID NO: 6 |
| | CLHQIQKNKEDIIELKGSAIGLPIYQL | |
| | NSKLVDLERKFQGLQQT | |
| hThrombos | LRGLRTIVTTLQDSIRKVTEENKELA | SEQ ID NO: 7 |
| pondins | NE | |

Another example of a trimerizing domain is disclosed in U.S. Pat. No. 6,190,886 (incorporated by reference herein in its entirety), which describes polypeptides comprising a collectin neck region. Trimers can then be made under appropriate conditions with three polypeptides comprising the collectin neck region amino acid sequence. A number of collectins are identified, including:

Collectin neck region of human SP-D:

[SEQ ID NO: 8]

VASLRQQVEALQGQVQHLQAAFSQYKK
Collectin neck region of bovine SP-D:

[SEQ ID NO: 9]

VNALRQRVGILEGQLQRLQNAFSQYKK
Collectin neck region of rat SP-D:

[SEQ ID NO: 11]

SAALRQQMEALNGKLQRLEAAFSRYKK
Collectin neck region of bovine conglutinin:

[SEQ ID NO: 12]

VNALKQRVTILDGHLRRFQNAFSQYKK
Collectin neck region of bovine collectin:

[SEQ ID NO: 13]

VDTLRQRMRNLEGEVQRLQNIVTQYRK
Neck region of human SP-D:

[SEQ ID NO: 14]

GSPGLKGDKGIPGDKGAKGESGLPDVASLRQQVEALQGQVQHLQAAFSQY
KKVELFPGGIPHRD

Other examples of a MBP trimerizing domain is described in PCT Application Serial No. U.S.08/76266, published as WO 2009/036349, which is incorporated by reference in its entirety. This trimerizing domain can oligomerize even further and create higher order multimeric complexes.

In the present context, the “trimerising domain” is capable of interacting with other, similar or identical trimerising domains. The interaction is of the type that produces trimeric proteins or polypeptides. Such an interaction may be caused by covalent bonds between the components of the trimerising domains as well as by hydrogen bond forces, hydrophobic forces, van der Waals forces, and salt bridges. The trimerising effect of trimerizing domain is caused by a coiled coil structure that interacts with the coiled coil structure of two other trimerizing domains to form a triple alpha helical coiled coil trimer that is stable even at relatively high temperatures. In various embodiments, for example a trimerizing domain based upon a tetranectin structural element, the complex is stable at least 60° C., for example in some embodiments at least 70° C.

The terms “C-type lectin-like protein” and “C-type lectin” are used to refer to any protein present in, or encoded in the genomes of, any eukaryotic species, which protein contains one or more CTLDs or one or more domains belonging to a subgroup of CTLDs (previously referred to at the carbohydrate recognition domain (the CRDs), which bind carbohydrate ligands. The definition specifically includes membrane attached C-type lectin-like proteins and C-type lectins, “soluble” C-type lectin-like proteins and C-type lectins lacking a functional transmembrane domain and variant C-type lectin-like proteins and C-type lectins in which one or more amino acid residues have been altered in vivo by glycosylation or any other post-synthetic modification, as well as any product that is obtained by chemical modification of C-type lectin-like proteins and C-type lectins.

The CTLD consists of roughly 120 amino acid residues and, characteristically, contains two or three intra-chain disulfide bridges. Although the similarity at the amino acid sequence level between CTLDs from different proteins is relatively low, the 3D-structures of a number of CTLDs have been found to be highly conserved, with the structural variability essentially confined to a so-called loop-region, often defined by up to five loops. Several CTLDs contain either one or two binding sites for calcium and most of the side chains which interact with calcium are located in the loop-region.

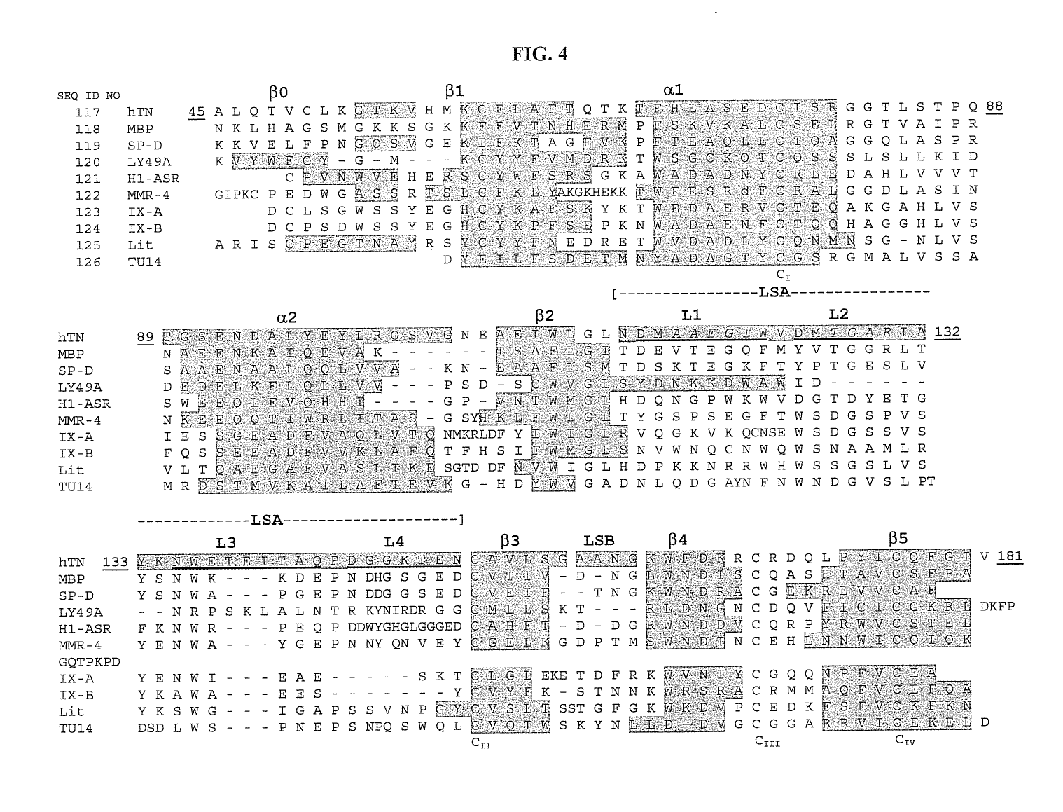

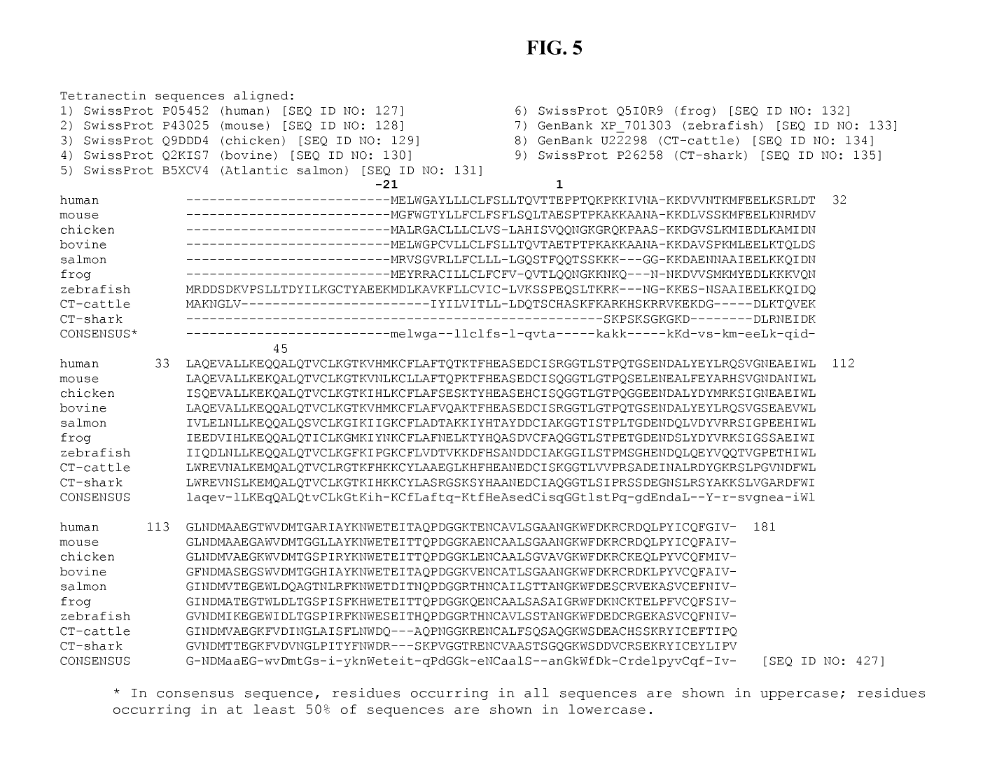

On the basis of CTLDs for which 3D structural information is available, it has been inferred that the canonical CTLD is structurally characterized by seven main secondary-structure elements (i.e. five β-strands and two α-helices) sequentially appearing in the order β1, α1, α2, β2, β3, β4, and β5. FIG. 4 illustrates an alignment of the CTLDs of known three dimensional structures of ten C-type lectins. In all CTLDs, for which 3D structures have been determined, the β-strands are arranged in two anti-parallel β-sheets, one composed of β1 and β5, the other composed of β2, β3 and β4. An additional β-strand, β0, often precedes β1 in the sequence and, where present, forms an additional strand integrating with the β1, β5-sheet. Further, two disulfide bridges, one connecting α1 and β5 (C_{I}-C_{IV}) and one connecting β3 and the polypeptide segment connecting β4 and β5 (C_{II}-C_{III}) are invariantly found in all CTLDs characterized to date. Also, FIG. 5 shows an alignment of CTLDs from human tetranectin and nine other tetranectin or tetranectin like polypeptides.

In the CTLD 3D-structure, these conserved secondary structure elements form a compact scaffold for a number of loops, which in the present context collectively are referred to as the “loop-region”, protruding out from the core. In the primary structure of the CTLDs, these loops are organized in two segments, loop segment A, LSA, and loop segment B, LSB. LSA represents the long polypeptide segment connecting β2 and β3 that often lacks regular secondary structure and contains up to four loops. LSB represents the polypeptide segment connecting the β-strands β3 and β4. Residues in LSA, together with single residues in β4, have been shown to specify the Ca²⁺—and ligand-binding sites of several CTLDs, including that of tetranectin. For example, mutagenesis studies, involving substitution of one or a few residues, have shown that changes in binding specificity, Ca²⁺-sensitivity and/or affinity can be accommodated by CTLDs. A number of CLTDs are known, including the following non-limiting examples: tetranectin, lithostatin, mouse macrophage galactose lectin, Kupffer cell receptor, chicken neurocan, perlucin, asialoglycoprotein receptor, cartilage proteoglycan core protein, IgE Fc receptor, pancreatitis-associated protein, mouse macrophage receptor, Natural Killer group, stem cell growth factor, factor IX/X binding protein, mannose binding protein, bovine conglutinin, bovine CL43, collectin liver 1, surfactant protein A, surfactant protein D, e-selectin, tunicate c-type lectin, CD94 NK receptor domain, LY49A NK receptor domain, chicken hepatic lectin, trout c-type lectin, HIV gp120-binding c-type lectin, and dendritic cell immunoreceptor. See U.S. Patent Publication No. 2007/0275393, which is incorporated herein by reference in its entirety.

In various embodiments of the invention, the amino acid sequence of the scaffold structure of the CLTD is at least 75% identical to the scaffold structures of the CTLD polypeptides of SEQ ID NOS: 15 and 117-135 (see FIGS. 4 and 5). In other embodiments, the scaffold structure is at least 80%, 85%, 90%, 95%, 96%, 97%, 98% or 99% identical to the scaffold structure of SEQ ID NOS: 15 and 117-135.

The expression “effective amount” refers to an amount of one or both of a death receptor agonist of the invention and a cytotoxic or immunosuppressive agent which is effective for preventing, ameliorating or treating the disease or condition in question whether administered simultaneously or sequentially. In particular embodiments, an effective amount is the amount of the death receptor agonist or death receptor binder, and a cytotoxic or immunosuppressive agent in combination sufficient to enhance, or otherwise increase the propensity (such as synergistically) of a cell to undergo apoptosis, reduce tumor volume, or prolong survival of a mammal having a cancer or immune related disease.

A “therapeutic agent” refers to a cytotoxic agent, a chemotherapeutic agent, an immunosuppressive agent, an immunostimulatory agent, and/or a growth inhibitory agent.

The term “immunosuppressive agent” as used herein for adjunct therapy refers to substances that act to suppress or mask the immune system of the mammal being treated herein. This would include substances that suppress cytokine production, downregulate or suppress self-antigen expression, or mask the MHC antigens. Examples of such agents include but are not limited to 2-amino-6-aryl-5-substituted pyrimidines (see U.S. Pat. No. 4,665,077); nonsteroidal antiinflammatory drugs (NSAIDs); azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g., prednisone, methylprednisolone, dexamethasone, and hydrocortisone; methotrexate (oral or subcutaneous); hydroxycloroquine; sulfasalazine; leflunomide; cytokine or cytokine receptor antagonists including anti-interferon-gamma (IFN-γ), -β, or -α antibodies, anti-tumor necrosis factor-α antibodies (infliximab or adalimumab), anti-TNFα immunoadhesin (etanercept), anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-13; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al., Science, 251: 430-432 (1991); WO 90/11294; Janeway, Nature, 341: 482 (1989); and WO 91/01133); and T-cell receptor antibodies (EP 340,109) such as T10B9.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma 11 and calicheamicin omega 11 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,22″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition are proteasome inhibitors such as bortezomib (Velcade), BCL-2 inhibitors, IAP antagonists (e.g. Smac mimics/xIAP and cIAP inhibitors such as certain peptides, pyridine compounds such as (S)—N-{6-benzo[1,3]dioxol-5-yl-1-[5-(4-fluoro-benzoyl)-pyridin-3-ylmethyl]-2-oxo-1,2-dihydro-pyridin-3-yl}-2-methylamino-propionamide, xIAP antisense), HDAC inhibitors (HDACI) and kinase inhibitors (Sorafenib).

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell, either in vitro or in vivo. Thus, the growth inhibitory agent is one that significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995, pg. 13).

Further included are agents that induce cell stress such as e.g. arginine depleting agents such as arginase.

Further included are targeted antibodies such as Rituximab. Furthermore, combinations of TRAIL agonists with aspirin and inhibitors of the NFkB pathway can be beneficial.

“Synergistic activity,” “synergy,” “synergistic effect,” or “synergistic effective amount” as used herein means that the effect observed when employing a combination of a TRAIL death receptor agonist and a therapeutic agent is (1) greater than the effect achieved when that TRAIL death receptor agonist or therapeutic agent is employed alone (or individually) and (2) greater than the sum added (additive) effect for that TRAIL death receptor agonist or therapeutic agent. Such synergy or synergistic effect can be determined by way of a variety of means known to those in the art. For example, the synergistic effect of a TRAIL death receptor agonist and a therapeutic agent can be observed in in vitro or in vivo assay formats examining reduction of tumor cell number or tumor mass.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured using well known art methods, for instance, by cell viability assays, FACS analysis or DNA electrophoresis, binding of annexin V, fragmentation of DNA, cell shrinkage, dilation of endoplasmic reticulum, cell fragmentation, and/or formation of membrane vesicles (called apoptotic bodies).

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer (NSCLC), gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

The term “immune related disease” means a disease or disorder in which a component of the immune system of a mammal causes, mediates or otherwise contributes to a morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

A “B-cell malignancy” is a malignancy involving B cells. Examples include Hodgkin's disease, including lymphocyte predominant Hodgkin's disease (LPHD); non-Hodgkin's lymphoma (NHL); follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLDi); Waldenstrom's macroglobulinemia (lymphoplasmacytic lymphoma); mucosa-associated lymphoid tissue (MALT) lymphoma; and marginal zone lymphoma/leukemia.

Non-Hodgkin's lymphoma (NHL) includes, but is not limited to, low grade/follicular NHL, relapsed or refractory NHL, front line low grade NHL, Stage III/IV NHL, chemotherapy resistant NHL, small lymphocytic (SL) NHL, intermediate grade/follicular NHL, intermediate grade diffuse NHL, diffuse large cell lymphoma, aggressive NHL (including aggressive front-line NHL and aggressive relapsed NHL), NHL relapsing after or refractory to autologous stem cell transplantation, high grade immunoblastic NHL, high grade lymphoblastic NHL, high grade small non-cleaved cell NHL, bulky disease NHL, etc.

Tumor-associated antigens (TAA) or tumor-specific antigens (TSA) are molecules produced in tumor cells that can trigger an immune response in the host. Tumor associated antigens are found on both tumor and normal cells, although at differential expression levels, whereas tumor specific antigens are exclusively expressed by tumor cells. TAAs or TSAs exhibiting on the surface of tumor cells include but are not limited to alfafetoprotein, carcinoembryonic antigen (CEA), CA-125, MUC-1, glypican-3, tumor associated glycoprotein-72 (TAG-72), epithelial tumor antigen, tyrosinase, melanoma associated antigen, MART-1, gp100, TRP-1, TRP-2, MSH-1, MAGE-1, -2, -3, -12, RAGE-1, GAGE 1-, -2, BAGE, NY-ESO-1, beta-catenin, CDCP-1, CDC-27, SART-1, EpCAM, CD20, CD23, CD33, EGFR, HER-2, breast tumor-associated antigens BTA-1 and BTA-2, RCAS1 (receptor-binding cancer antigen expressed on SiSo cells), PLACenta-specific 1 (PLAC-1), syndecan, MN (gp250), idiotype, among others. Tumor associated antigens also include the blood group antigens, for example, Le^{a}, Le^{b}, LeX, LeY, H-2, B-1, B-2 antigens. (See Table 18 at the end of the specification). Ideally, for the purposes of this invention, TAA or TSA targets do not get internalized upon binding.

Turning now to the invention in more detail, in one aspect the invention is directed to a non-natural polypeptide comprising a multimerizing domain that includes at least one polypeptide binding member that binds to at least one TRAIL death receptor. In accordance with the invention, the binding member may either be linked to the N- or the C-terminal amino acid residue of the multimerizing domain. Also, in certain embodiments it may be advantageous to link a binding member to both the N-terminal and the C-terminal amino acid of the multimerizing domain of the monomer, and thereby providing a multimeric polypeptide complex comprising, for example, up to six binding members (when the multimierizing domain is a trimerizing domain) capable of binding a TRAIL death receptor. The polypeptides of the invention are non-natural polypeptides, for example, fusion proteins of a multimerizing domain and a polypeptide sequence that binds a TRAIL death receptor. The non-natural polypeptides may also be natural polypeptides wherein the naturally occurring amino acid sequence has been altered by the addition, deletion, or substitution of amino acids. Examples of such polypeptide include polypeptides having a C-type Lectin Like Domain (CTLD) wherein one or more of the loop regions of the domains have been modified as described herein. Naturally occurring binders for TRAIL death receptors are not non-natural polypeptides within the scope of the invention. In this aspect of the invention, the trimerizing domain is not a sequence that can be obtained from, and has no substantial homology to, a naturally occurring polypeptide that binds to a TRAIL death receptor (e.g., TRAIL). In other aspects of the invention, the polypeptide that binds to at least one TRAIL death receptor is a fragment or variant of a natural polypeptide that binds to a death receptor, wherein when the naturally occurring polypeptide, variant or fragment is fused to a multimerizing domain, the fusion protein is no longer a naturally occurring polypeptide. Accordingly, the invention does not exclude naturally occurring polypeptide, fragments or variants thereof from being a part of fusion protein of the invention.

In various aspects of the invention, the multimerizing domain is a trimerizing domain, such as the non-limiting examples described herein.

In an embodiment of this aspect, the polypeptide binds to a TRAIL death receptor that activates apoptosis in a tumor cell. In one embodiment polypeptide binds to TRAIL-R1 (DR4) (SEQ ID NO: 137) or TRAIL-R2 (DR5) (SEQ ID NO: 138) or conservative substitution variants thereof. In a particular embodiment, the polypeptide does not specifically bind to at least one TRAIL decoy receptor.

In various aspects, a monomeric polypeptide includes at least two segments: a multimerizing domain that is capable of forming a multimeric complex with other multimerizing domains, and a polypeptide sequence that binds to at least one TRAIL death receptor. The sequence that binds to a TRAIL death receptor may be fused with the multimerizing domain at the N-terminus, at the C-terminus, or at both the N- and C-termini of the domain.

In one embodiment, a first polypeptide that binds TRAIL-R1 (DR4) (SEQ ID NO: 137) or TRAIL-R2 (DR5) (SEQ ID NO: 138) is fused at one of the N-terminus and the C-terminus of a trimerizing domain, and a second polypeptide that binds TRAIL-R1 (DR4) (SEQ ID NO: 137) or TRAIL-R2 (DR5) (SEQ ID NO: 138) is fused at the other of the N-terminus or the C-terminus of the trimerizing domain.

In a further embodiment, both of the first and second polypeptides bind TRAIL-R1 (DR4) (SEQ ID NO: 137) or both the first and second polypeptides bind TRAIL-R2 (DR5) (SEQ ID NO: 138). In even a further embodiment, the first polypeptide binds TRAIL-R1 (DR4) (SEQ ID NO: 137), and the second polypeptide binds TRAIL-R2 (DR5) (SEQ ID NO: 138). Advantages of a bi-specific molecules that target both receptors is greater potency and greater coverage due to differential expression with some patients expressing both DR4 and DR5 and with other patients expressing either one or the other. Also, it is expected that the bi-specific molecules would effect super-clustering via tumor cell specific binding on both ends of the molecule, i.e., super-clustering effects mediated in both directions.

Since TRAIL receptors are fairly broadly expressed across human tissues, another aspect of the invention includes a trimerizing domain having a polypeptide that binds to either DR4 or DR5 on one end of the domain (one of either of the N-terminus or C-terminus), and a polypeptide that binds to tumor-associated (TAA) or tumor-specific antigens (TSA) on the other end (the other of the N-terminus and the C-terminus). The domain that binds to TAA's or TSA's may be peptides, such as for example CTLDs, single chain antibodies, or any type of domain that specifically binds to the desired target. In these cases, agonist activity to a target that promotes apoptosis would be significantly enhanced with superclustering mediated by multiple trimerized complexes binding to TAA or TSA's on a given tumor cell surface and interacting with another tumor cell in the vicinity. In addition, the tumor specific peptide binding domain can direct the drug (bound to the trimerized complex) to the tumor site, thereby making the tumor killing activity more specific, and can improve target residence time through tumor specificity. Improved tumor penetration due to smaller size compared to an antibody (˜70 kD vs. 150 kD), along with improved target residence time through avidity benefits (three binding arms in close proximity vs. two) are expected to provide additional efficacy and safety advantages.

In one particular approach the potential risk of toxicity on normal tissues can be reduced by designing a molecule with weak agonist activity mediated through a DR4- or DR5-binding polypeptide one end of a trimerizing domain that improves clustering that is mediated through the tumor-specific polypeptide on the second end of the trimerizing domain. In various aspects, the polypeptide binds to a death receptors at lower affinity than to a TAA or TSA. More specifically, the polypeptide binds the binds the TAA or TSA with least 2 times greater affinity, for example, 2, 2.5, 3, 3.5, 4, 4.5 5, 10, 15, 20, 50 and 100 times greater, than the polypeptide binds the death receptor.

Higher affinity on the tumor antigen-targeting site could potentially also enhance potency through prevention of TRAIL-receptor internalization while bound to both a TRAIL receptor and a TAA or TSA targeting agent. Similarly, combination therapy or chemical linkage to a death receptor agonist with an agent preventing internalization, such as chlorpromazine, could enhance potency of the TRAIL receptor agonist (see, Zhang, et al., Mol. Cancer. Res. (2008) 6:1861-72).

In one aspect, the invention is directed to polypeptides that bind one or more TRAIL death receptors but are not agonists for the receptors. Polypeptides binding to DR4/DR5 but lacking agonist activity are used to deliver a payload thereby killing cancer cells. DR4/DR5 receptors are internalized (Kohlhaas, J Biol. Chem. 2007 Apr. 27; 282(17):12831-41).

Furthermore, potency of TRAIL receptor agonists can be enhanced by targeting death receptors that work synergistically with the TRAIL receptor by providing bispecific molecules having a DR4 or DR5 agonist at one end of a trimerizing domain and a TNF receptor agonist, an FN14 agonist, FAS receptor agonist, LIGHT receptor agonist on the other end of the trimerizing domain. (See Table 17 at the end of the specification).

Indications for trimeric complexes having both TRAIL receptor-binding polypeptide(s) and TAA or TSA targeting agent(s) include non-small cell lung cancer (NSCLC), colorectal cancer, ovarian cancer, renal cancer, pancreatic cancer, sarcomas, non-hodgkins lymphoma (NHL), multiple myeloma, breast cancer, prostate cancer, melanoma, glioblastoma, neuroblastoma.

In addition, while normal cells do not display phosphatidylserine on the cell surface, cells undergoing apoptosis flip phosphatidylcholine to phosphatidylserine on the surface. Therefore, apoptotic cells can be targeted by phosphatidylserine-binding agents. Phosphatidylserine binding agents include but are not limited to antibodies, antibody fragments, CTLDs or peptides as, for example, described by Burtea et al (Mol. Pharm. 2009 Sep. 10 [published online ahead of print]). Molecules with DR4 and/or DR5 agonist activity on one end and phosphotidylserine targeting peptides in the other end would result in better tumor targeting of the DR agonists as well as potentially enhance potency through cross-linking.

In another aspect, a polypeptide that specifically binds to a TRAIL death receptor is contained in the loop region of a CTLD. The polypeptide may be a TRAIL polypeptide, or may be sequence that is identified as provided here, but is not a naturally occurring TRAIL sequence or fragment thereof, and is not a TRAIL polypeptide as described herein. In this aspect the sequence is contained in a loop region of a CLTD, and the CTLD is fused to a trimerizing domain at the N-terminus or C-terminus of the domain either directly or through the appropriate linker. Also, the polypeptide of the invention may include a second CLTD domain, fused at the other of the N-terminus and C-terminus. In a variation of this aspect, the polypeptide includes a polypeptide that binds to a TRAIL death receptor at one of the termini of the trimerizing domain and a CLTD at the other of the termini. One, two or three of the polypeptides can be part of a trimeric complex containing up to six specific binding members for a TRAIL death receptor.

The polypeptides of the invention can include one or more amino acid mutations in a native TRAIL sequence, or a random sequence, that has selective binding affinity for either the DR4 receptor or the DR5 receptor, but not a TRAIL decoy receptor. In another embodiment, the TRAIL variant or the random sequence has a selective binding affinity for both DR4 and DR5, but not a TRAIL decoy receptor. In various embodiments, the sequence selectively binds DR4, but not DR5 and a decoy receptor. In a similar embodiment, the sequence binds DR5, but not DR4 and a decoy receptor.

The polypeptide sequences that bind one or more TRAIL death receptors can have a binding affinity for DR4 and/or DR5 that is about equal to the binding affinity that native TRAIL has for the death receptor(s). In certain embodiments, the polypeptides of the invention have a binding affinity for one or more TRAIL death receptor(s) that is greater than the binding affinity that native TRAIL has for the same TRAIL death receptor(s).

In one aspect the TRAIL death receptor agonists of the invention are selective for the DR4 and DR5 receptors. For example, when binding affinity of such binding members to the DR4 or DR5 receptor is approximately equal (unchanged) or greater than (increased) as compared to native sequence TRAIL, and the binding affinity of the binding member to a decoy receptor is less than or nearly eliminated as compared to native sequence TRAIL, the binding affinity of the binding member, for purposes herein, is considered “selective” for the DR4 or DR5 receptor. In another example, the affinity of the binding member for a death receptor is less than the affinity of TRAIL for the same receptor, but the binding member is still selective for the receptor if it has greater affinity for a death receptor than a decoy receptor. Preferred DR4 and DR5 selective agonists of the invention will have at least 5-fold, preferably at least a 10-fold greater binding affinity to a death receptor as compared to a decoy receptor, and even more preferably, will have at least 100-fold greater binding affinity to a death receptor as compared to a decoy receptor. The binding members may have different binding affinity for DR4 and DR5.

The respective binding affinity of the agonists can be determined and compared to the binding properties of native TRAIL, or a portion thereof, by ELISA, RIA, and/or BIAcore assays, known in the art. Preferred DR4 and DR5 selective agonists of the invention will induce apoptosis in at least one type of mammalian cell (e.g., a cancer cell), and such apoptotic activity can be determined by known art methods such as the alamar blue or crystal violet assay.

In an embodiment, the TRAIL death receptor agonist comprises an antibody or an antibody fragment. In the present context, the term “antibody” is used to describe an immunoglobulin whether natural or partly or wholly synthetically produced. As antibodies can be modified in a number of ways, the term “antibody” should be construed as covering any specific binding member or substance having a binding domain with the required receptor specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. The term also covers any polypeptide or protein having a binding domain which is, or is homologous to, an antibody binding domain, e.g. antibody mimics. These can be derived from natural sources, or they may be partly or wholly synthetically produced. Examples of antibodies are the immunoglobulin isotypes and their isotypic subclasses; fragments which comprise an antigen binding domain such as Fab, Fab′, F(ab′)₂, scFv, Fv, dAb, Fd; and diabodies.

In another aspect the invention relates to a multimeric complex of three polypeptides, each of the polypeptides comprising a multimerizing domain and at least one polypeptide that binds to at least one TRAIL death receptor. In an embodiment, the multimeric complex comprises a polypeptide having a multimerizing domain selected from a polypeptide having substantial homology to a human tetranectin trimerizing structural element or other human trimerizing polypeptides including mannose binding protein (MBP) trimerizing domain, a collectin neck region polypeptide, and others. The multimeric complex can be comprised of any of the polypeptides of the invention wherein the polypeptides of the multimeric complex comprise multimerizing domains that are able to associate with each other to form a multimer. Accordingly, in some embodiments, the multimeric complex is a homomultimeric complex comprised of polypeptides having the same amino acid sequences. In other embodiments, the multimeric complex is a heteromultimeric complex comprised of polypeptides having different amino acid sequences such as, for example, different multimerizing domains, and/or different polypeptides that bind to a TRAIL death receptor. In such embodiments, the polypeptides that specifically bind to a TRAIL death receptor may be targeted to the same TRAIL death receptor. In other embodiments, the polypeptides that specifically bind to a TRAIL death receptor are targeted to the different TRAIL death receptors, for example, DR4 and DR5. Thus, in certain embodiments the multimeric complex comprises polypeptides of the invention, wherein each of the polypeptides comprise at least one polypeptide that binds to DR4, wherein the DR4-binding polypeptides can be the same or different, and/or at least one polypeptide that binds to DR5, wherein the DR5-binding polypeptides can be the same or different.

Further, in one aspect, the invention relates to a method for preparing a polypeptide that induces apoptosis in a cell expressing at least one death receptor for TRAIL comprising: (a) selecting a first polypeptide(s) that specifically binds one of DR4 or DR5 but does not bind a TRAIL decoy receptor; (b) grafting the first polypeptide(s) into one or two loop segments of tetranectin CTLD to form a first binding determinant (c) fusing the first CTLD with one of the N-terminus or the C-terminus of a tetranectin trimerizing structural element. In another embodiment of this aspect, the method further comprises (a) selecting a second polypeptide(s) that is selected to specifically bind the other of DR4 and DR5 relative to the first polypeptide; (b) grafting the second polypeptide(s) into a loop region of a tetranectin CTLD to form a second binding determinant; and (c) fusing the second CTLD with the other of the N-terminus or the C-terminus of the tetranectin trimerizing structural element. In other embodiments, the first and second polypeptides can be directly fused to the trimerizing domain.

The tetranectin CTLD has up to five loop regions into which binding members for TRAIL death receptors may be inserted. Accordingly, when a polypeptide of the invention includes a CTLD, the polypeptide may have up to four or five binding members for TRAIL death receptors attached to the trimerizing domain through the CTLD. Each of the binding members may be the same or different, and may be agonists for either DR4 or DR5, or both.

In other aspects of the polypeptides of the invention, a receptor agonist can be bound to one terminus of a trimerizing domain and one or more therapeutic agents may be bound to the second terminus. The agent may be bound directly or through an appropriate linker as understood to those of skill in the art. Such agents may act in the same apoptotic pathway as the agonist, or may act in a different pathway for treating cancer and other conditions. Also, such agents may upregulate DR4 and DR5 expression. In addition to being bound to one of the termini of the polypeptides, the agent may be covalently linked to the trimerizing domain via a peptide bond to a side chain in the trimerizing domain or via a bond to a cysteine residue. Other ways of covalently coupling the agent to the module can also be used as show in, for example, U.S. Pat. No. 6,190,886, which is incorporated by reference herein.

Identification of Polypeptide Sequences Specific for TRAIL Death Receptors

In one aspect, a specific binding member for a TRAIL death receptor can be obtained from a random library of polypeptides by selection of members of the library that specifically bind to the receptor. A number of systems for displaying phenotypes with putative ligand binding sites are known. These include: phage display (e.g. the filamentous phage fd [Dunn (1996), Griffiths and Duncan (1998), Marks et al. (1992)], phage lambda [Mikawa et al. (1996)]), display on eukaryotic virus (e.g. baculovirus [Ernst et al. (2000)]), cell display (e.g. display on bacterial cells [Benhar et al. (2000)], yeast cells [Boder and Wittrup (1997)], and mammalian cells [Whitehorn et al. (1995)], ribosome linked display [Schaffitzel et al. (1999)], and plasmid linked display [Gates et al. (1996)].

Also, US2007/0275393, which is incorporated herein by reference in its entirety, specifically describes a procedure for accomplishing a display system for the generation of CLTD libraries. The general procedure includes (1) identification of the location of the loop-region, by referring to the 3D structure of the CTLD of choice, if such information is available, or, if not, identification of the sequence locations of the β2, β3 and β4 strands by sequence alignment with known sequences, as aided by the further corroboration by identification of sequence elements corresponding to the β2 and β3 consensus sequence elements and β4-strand characteristics, also disclosed above; (2) subcloning of a nucleic acid fragment encoding the CTLD of choice in a protein display vector system with or without prior insertion of endonuclease restriction sites close to the sequences encoding β2, β3 and β4; and (3) substituting the nucleic acid fragment encoding some or all of the loop-region of the CTLD of choice with randomly selected members of an ensemble consisting of a multitude of nucleic acid fragments which after insertion into the nucleic acid context encoding the receiving framework will substitute the nucleic acid fragment encoding the original loop-region polypeptide fragments with randomly selected nucleic acid fragments. Each of the cloned nucleic acid fragments, encoding a new polypeptide replacing an original loop-segment or the entire loop-region, will be decoded in the reading frame determined within its new sequence context.

A complex may be formed that functions as a homo-trimeric protein, signaling through the TRAIL-R1 (DR4) and TRAIL-R2 (DR5) receptors to induce apoptosis. Since trimerization of these receptors by the TRAIL ligand is involved in the formation of the death-induced signaling complex (DISC) and subsequent full induction of the apoptotic signaling pathway, the trimeric structure of the human tetranectin protein presents a uniquely ideal scaffold in which to construct libraries with members capable of binding to the TRAIL-R1 and TRAIL-R2 receptors and inducing trimerization of the receptors and agonist activity. However peptides with TRAIL receptor binding activity must be identified first. To accomplish this, peptides with known binding activity can be used or additional new peptides identified by screening from display libraries. A number of different display systems are available, such as but not limited to phage, ribosome and yeast display.

To select for new peptides with binding activity, libraries can be constructed and initially screened for binding to the TRAIL receptors as monomeric elements, either as single monomeric CTLD domains, or individual peptides displayed on the surface of phage. Once sequences with TRAIL receptor binding activity have been identified these sequences would subsequently be grafted on to the trimerization domain of human tetranectin to create potential protein therapeutics capable of binding three receptors in a trimeric complex to induce agonist activity (apoptosis).

Four main strategies may be employed in the construction of these phage display libraries and trimerization domain constructs. The first strategy would be to construct and/or use random peptide phage display libraries. Random linear peptides and/or random peptides constructed as disulfide constrained loops would be individually displayed on the surface of phage particles and selected for binding to the desired TRAIL receptor through phage display “panning”. After obtaining peptide clones with TRAIL receptor binding activity, these peptides would be grafted on to the trimerization domain of human tetranectin or into loops of the CTLD domain followed by grafting on the trimerization domain and screened for agonist activity.

A second strategy for construction of phage display libraries and trimerization domain constructs would include obtaining CTLD derived binders. Libraries can be constructed by randomizing the amino acids in one or more of the five different loops within the CTLD scaffold of human tetranectin displayed on the surface of phage. Binding to the TRAIL receptors can be selected for through phage display panning After obtaining CTLD clones with peptide loops demonstrating TRAIL receptor binding activity, these CTLD clones can then be grafted on to the trimerization domain of human tetranectin and screened for agonist activity.

A third strategy for construction of phage display libraries and trimerization domain constructs would includes taking known sequences with binding capabilities to the TRAIL receptors and graft these directly on to the trimerization domain of human tetranectin and screen for agonist activity.

A fourth strategy includes using peptide sequences with known binding capabilities to the TRAIL receptors and first improve their binding by creating new libraries with randomized amino acids flanking the peptide or/and randomized selected internal amino acids within the peptide, followed by selection for improved binding through phage display. After obtaining binders with improved affinity, the binders of these peptides can be grafted on to the trimerization domain of human tetranectin and screening for agonist activity. In this method, initial libraries can be constructed as either free peptides displayed on the surface of phage particles, as in the first strategy (above), or as constrained loops within the CTLD scaffold as in the second strategy also discussed above. After obtaining binders with improved affinity, grafting of these peptides on to the trimerization domain of human tetranectin and screening for agonist activity would occur.

Truncated versions of the trimerization domain can be used that either eliminate up to 16 residues at the N-terminus (V17), or alter the C-terminus. C-terminal variations termed Trip V [SEQ ID NO: 76], Trip T [SEQ ID NO: 77], Trip Q [SEQ ID NO: 78] and Trip K [SEQ ID NO: 75] See FIG. 3) allow for unique presentation of the CTLD domains on the trimerization domain. The TripK variant is the longest construct and contains the longest and most flexible linker between the CTLD and the trimerization domain. Trip V, Trip T, Trip Q represent fusions of the CTLD molecule directly onto the trimerization module without any structural flexibility but are turning the CTLD molecule 1/3^{rd} going from TripV to TripT and from TripT to TripQ. This is due to the fact that each of these amino acids is in an α-helical turn and 3.2 aa are needed for a full turn. Free peptides selected for binding in the first, third and fourth strategies can be grafted onto any of above versions of the trimerization domain. Resulting fusions can then be screened to see which combination of peptide and orientation gives the best activity. Peptides selected for binding constrained within the loops of the CTLD of tetranectin can be grafted on to the full length trimerization domain.

More particularly, the four strategies are described below. Although these strategies focus on phage display, other equivalent methods of identifying polypeptides can be used.

Strategy 1

Peptide display library kits such as, but not limited to, the New England Biolabs Ph.D. Phage display Peptide Library Kits are sold commercially and can be purchased for use in selection of new and novel peptides with TRAIL receptor binding activity. Three forms of the New England Biolabs kit are available: the Ph.D.-7 Peptide Library Kit containing linear random peptides 7 amino acids in length, with a library size of 2.8×10⁹ independent clones, the Ph.D.—C7C Disulfide Constrained Peptide Library Kit containing peptides constructed as disulfide constrained loops with random peptides 7 amino acids in length and a library size of 1.2×10⁹ independent clones, and the Ph.D.—12 Peptide Library Kit containing linear random peptides 12 amino acids in length, with a library size of 2.8×10⁹ independent clones.

Alternatively similar libraries can be constructed de novo with peptides containing random amino acids similar to these kits. For construction random nucleotides are generated using either an NNK, or NNS strategy, in which N represents an equal mixture of the four nucleic acid bases A, C, G and T. The K represents an equal mixture of either G or T, and S represents and equal mixture of either G or C. These randomized positions can be cloned onto to the Gene III protein in either a phage or phagemid display vector system. Both the NNK and the NNS strategy cover all 20 possible amino acids and one stop codon with slightly different frequencies for the encoded amino acids. Because of the limitations of bacterial transformation efficiency, library sizes generated for phage display are in the order of those started above, thus peptides containing up to 7 randomized amino acids positions can be generated and yet cover the entire repertoire of theoretical combinations (20⁷=1.28×10⁹). Longer peptide libraries can be constructed using either the NNK or NNS strategy however the actual phage display library size likely will not cover all the theoretical amino acid combinations possible associated with such lengths due to the requirement for bacterial transformation.

Thus ribosome display libraries might be beneficial where larger/longer random peptides are involved. For disulfide constrained libraries a similar NNK or NNS random nucleotide strategy is used. However, these random positions are flanked by cysteine amino acid residues, to allow for disulfide bridge formation. The N terminal cysteine is often preceded by an additional amino acid such as alanine. In addition a flexible linker made up to but not limited to several glycine residues may act as a spacer between the peptides and the gene III protein for any of the above random peptide libraries.

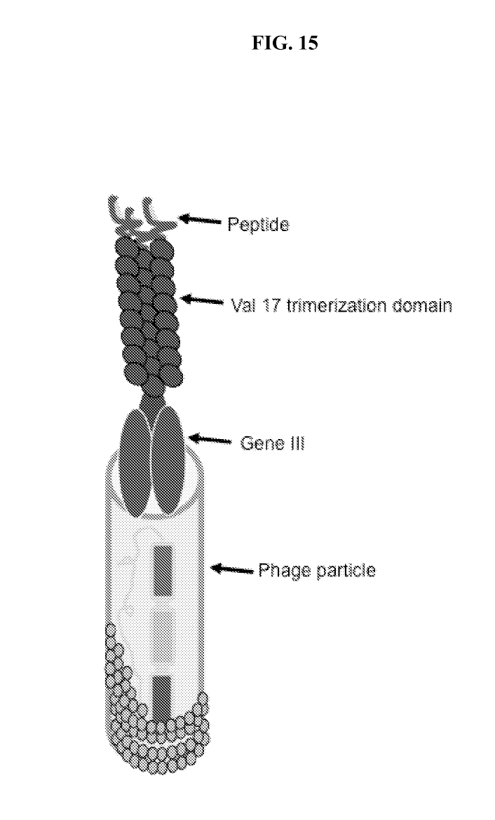

For example, in order to select for peptides which would bind in a trimeric conformation when fused to the trimerization domain of human tetranectin, a peptide phage display library can be constructed. In this library the C-terminus of the trimerization domain is fused to the N terminus of gene III of the phage with an amber stop codon at the junction. This allows for both the trimerization domain/gene III fusion protein as well as the trimerization domain alone to be produced, so that a trimeric protein fused through a single gene III coat protein could be assembled and displayed on the surface of the phage particle. In addition the N terminus of the trimerization domain is fused with a peptide consisting of 15 random amino acids, thus allowing the random peptide library to be displayed at a trimer (see FIG. 15).

Strategy 2

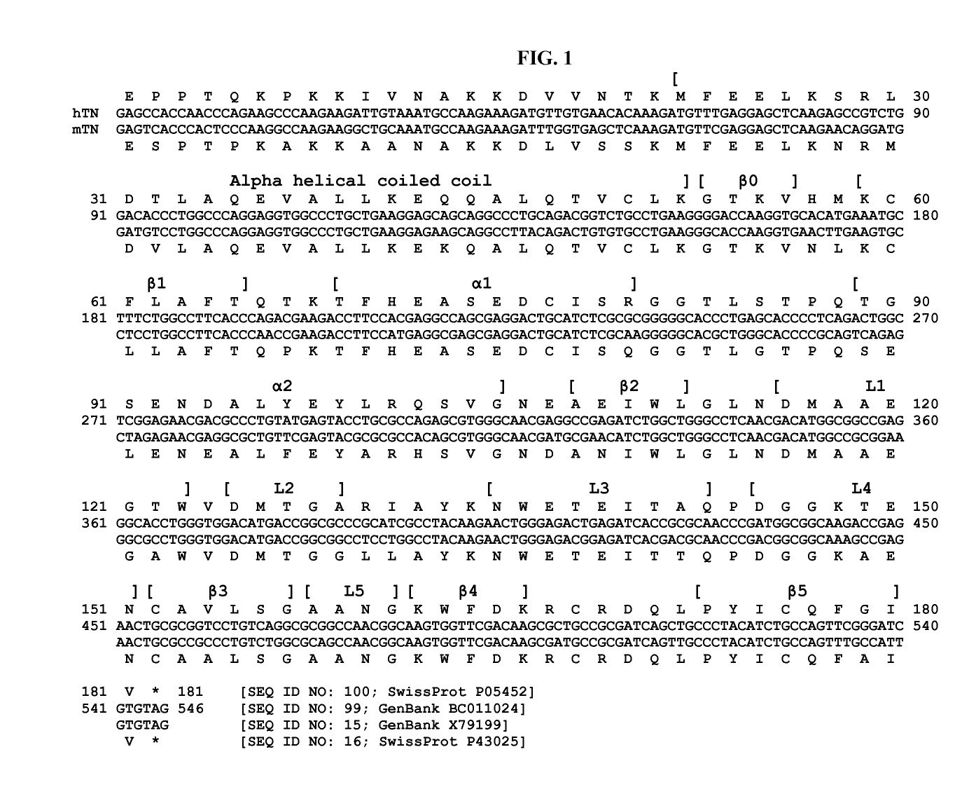

The human tetranectin CTLD shown in FIGS. 1 and 4 contains five loops (four loops in LSA and one loop comprising LSB), which can be altered to confer binding of the CTLD to different proteins targets. Random amino acid sequences can be placed in one or more of these loops to create libraries from which CTLD domains with the desired binding properties can be selected. Construction these libraries containing random peptides constrained within any or all of the five loops of the human tetranectin CTLD can be accomplished (but is not limited to) using either a NNK or NNS as described above in strategy 1. A single example of a method by which seven random peptides can be inserted into loop 1 of the TN CTLD is as follows.

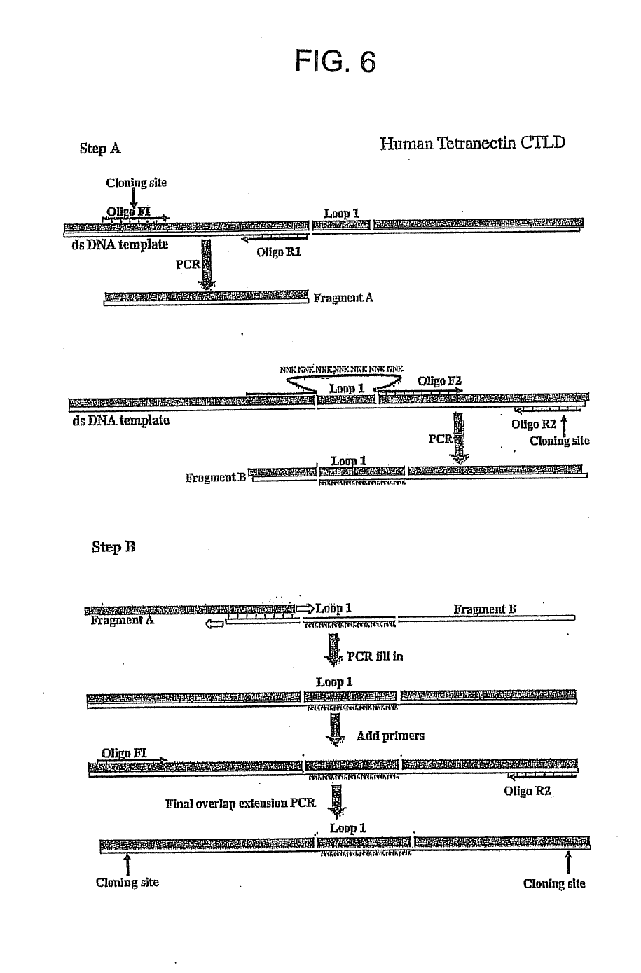

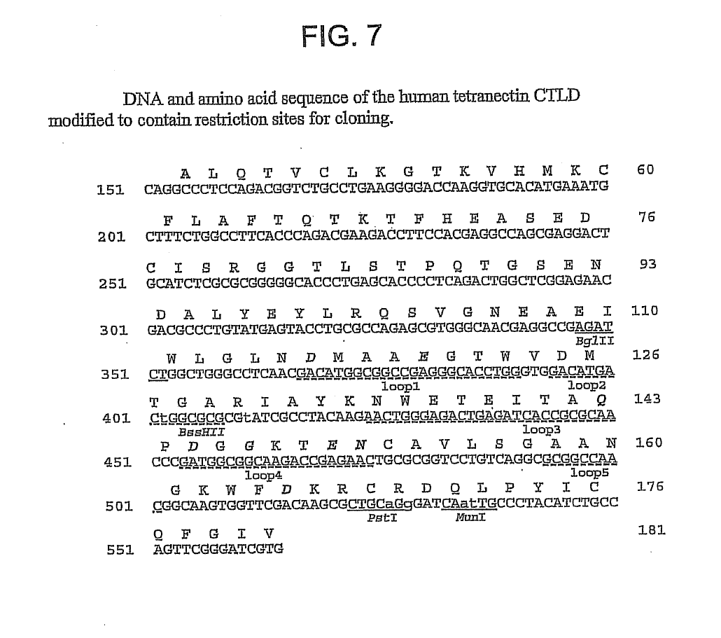

PCR of fragment A can be performed using the forward oligoF1 (5′-GCC CTC CAG ACG GTC TGC CTG AAG GGG-3′; SEQ ID NO: 171) which binds to the N terminus of the CTLD; the reverse oligo R1 (5′-GTT GAG GCC CAG CCA GAT CTC GGC CTC-3′; SEQ ID NO: 172) which binds to the DNA sequence just 5′ to loop 1. Fragment B can be created using forward oligo F2 (5′-GAG GCC GAG ATC TGG CTG GGC CTC AAC NNK NNK NNK NNK NNK NNK NNK TGG GTG GAC ATG ACC GGC GCG CGC ATC-3′; SEQ ID NO: 173) and the reverse primer R2 (5′-CAC GAT CCC GAA CTG GCA GAT GTA GGG-3′; SEQ ID NO: 174). The forward primer F2 has a 5′-end that is complementary to primer R1, and replaces the first 7 amino acids of loop 1 with random amino acids, and contains a 3′ end which binds to last amino acid of loop 1 and the sequences 3′ of it, while the reverse primer R2 is complementary and binds to the end of the CTLD sequences (see FIG. 6). PCR can be performed using a high fidelity polymerase or taq blend and standard PCR thermocycling conditions. Fragments A and B can then be gel isolated and then combined for overlap extension PCR using the primers F1 and R2 as described above. Digestion with the restriction enzymes Bgl II and PstI can allow for isolation of the fragment containing the loops of the TN CTLD and subsequent ligation into a phage display vector (such as CANTAB 5E) containing the restriction modified CTLD shown below fused to Gene III, which is similarly digested with Bgl II and Pst I for cloning. (See FIG. 7).

Modification of other loops by replacement with randomized amino acids can be similarly performed as shown above. The replacement of defined amino acids within a loop with randomized amino acids is not restricted to any specific loop, nor is it restricted to the original size of the loops. Likewise, total replacement of the loop is not required, partial replacement is possible for any of the loops. In some cases retention of some of the original amino acids within the loop, such as the calcium coordinating amino acids shown in FIG. 4 may be desirable. In these cases, replacement with randomized amino acids may occur for either fewer of the amino acids within the loop to retain the calcium coordinating amino acids, or additional randomized amino acids may be added to the loop to increase the overall size of the loop yet still retain these calcium coordinating amino acids. Very large peptides can be accommodated and tested by combining loop regions such as loops 1 and 2 or loops 3 and 4 into one larger replacement loop. In addition, other CTLDs, such as but not limited to the Mannose Binding Lectin CTLD, can be used instead of the CTLD of tetranectin. Grafting of peptides into these CTLDs can occur using methods similar to those described above. See, e.g., U.S. patent application Ser. No. 12/703,752, U.S Patent Application Publication No. 2011/0086770, which is incorporated by reference herein in its entirety.

In various exemplary aspects of the invention, the polypeptides that bind to a TRAIL death receptor can be identified using a combinatorial peptide library, and a library of nucleic acid sequences encoding the polypeptides of the library, based upon a CTLD backbone, wherein the CTLDs of the polypeptides have been modified according to a number of exemplary schemes, which have been labeled for the purposes of identification only as Schemes (a)-(g):
(a) one or more acid modifications in at least one of four loops in loop segment A (LSA) of the CTLD, wherein the one or more amino acid modifications comprises an insertion of at least one amino acid in Loop 1 and random substitution of at least five amino acids within Loop 1;
(b) one or more amino acid modifications in at least one of four loops in loop segment A (LSA) of the CTLD, wherein the one or more amino acid modifications comprises random substitution of at least five amino acids within Loop 1, and random substitution of at least three amino acids within Loop 2;
(c) one or more amino acid modifications in at least one of four loops in the loop segment A (LSA) of the CTLD, wherein the one or more amino acid modifications comprises random substitution of at least seven amino acids within Loop 1 and at least one amino acid insertion in Loop 4;
(d) one or more amino acid modifications in at least one of four loops in the loop segment A (LSA) of the CTLD, wherein the one or more amino acid modifications comprises at least one amino acid insertion in Loop 3 and random substitution of at least three amino acids within Loop 3;
(e) one or more amino acid modifications in at least one of four loops in the loop segment A (LSA) of the CTLD, wherein the one or more amino acid modifications comprises a modification that combines two loops into a single loop, wherein the two combined loops are Loop 3 and Loop 4;
(f) one or more amino acid modifications in at least one of four loops in the loop segment A (LSA) of the CTLD, wherein the one or more amino acid modifications comprises at least one amino acid insertion in Loop 4, and random substitution of at least three amino acids within Loop 4; of
(g) one or more amino acid modifications in at least one of five loops in the loop segment A (LSA) of the CTLD and loop segment B (LSB), wherein the one or more amino acid modifications comprises random substitution of five amino acid residues in Loop 3, and random substitution of at least three amino acids within Loop 5.

Accordingly, in an aspect, the invention relates to a combinatorial polypeptide library of polypeptide members having a modified C-type lectin domain (CTLD), wherein the modified CTLD includes one or more amino acid modifications in at least one of the four loops in LSA or in the LSB loop of the CTLD (loop 5), wherein the one or more amino acid modifications comprises an insertion of at least one amino acid in Loop 1 and random substitution of at least five amino acids within Loop 1.

In embodiments of this aspect the combinatorial library when the CTLD is from human tetranectin, the CTLD also has a random substitution of Arginine-130. For CTLDs other than the CTLD of human tetranectin, this peptide is located immediate adjacent the C-terminal peptide of Loop 2 in the C-terminal direction. For example, in mouse tetranectin, this peptide is Gly-130. In embodiments of this aspect the combinatorial library of CTLDs from human or mouse tetranectin, the CTLD includes a substitution of Lysine-148 to Alanine in Loop 4. In certain embodiments of this aspect the combinatorial library comprises two amino acid insertions in Loop 1, random substitution of at least five amino acids within Loop 1, random substitution of Arginine-130 or other amino acid located outside and adjacent to loop 2 in the C-terminal direction, and a substitution of Lysine-148 to Alanine in Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises random substitution of at least five amino acids within Loop 1, random substitution of at least three amino acids within Loop 2, and random substitution of Arginine-130, or other amino acid located outside and adjacent to loop 2 in the C-terminal direction and a substitution of Lysine-148 to Alanine in Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises random substitution of at least seven amino acids within Loop 1 and at least one amino acid insertion in Loop 4.

In embodiments of this aspect, the combinatorial library further comprises random substitution of at least two amino acids within Loop 4. In certain embodiments the combinatorial library comprises random substitution of at least seven amino acids within Loop 1, three amino acid insertions in Loop 4, and random substitution of at least two amino acids within Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises random substitution of at least six amino acids within Loop 3, for example 3, 4, 5, 6 or more, and, optionally, a substitution of Lysine-148 to Alanine in Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises at least one amino acid insertion in Loop 3 and random substitution of at least three amino acids within Loop 3 and a substitution of Lysine-148 to Alanine in Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises at least one amino acid insertion in Loop 3 and random substitution of at least six amino acids within Loop 3 and a substitution of Lysine-148 to Alanine in Loop 4.

In embodiments of this aspect, the combinatorial library further comprises at least one amino acid insertion in Loop 4. In certain embodiments the combinatorial library further comprises random substitution of at least three amino acids within Loop 4. In certain embodiments the combinatorial library comprises three amino acid insertions in Loop 3. In certain embodiments the combinatorial library further comprises three amino acid insertions in Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises a modification that combines two Loops into a single Loop, wherein the two combined Loops are Loop 3 and Loop 4.

In an embodiment of this aspect, the combinatorial library comprises the sequence NWEXXXXXXX XGGXXXN (SEQ ID NO: 175), wherein X is any amino acid and wherein the amino acid sequence forms a single loop from combined and modified Loop 3 and Loop 4.

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, wherein the one or more amino acid modifications comprises at least one amino acid insertion in Loop 4, and random substitution of at least three amino acids within Loop 4.

In an embodiment of this aspect, the combinatorial library comprises four amino acid insertions in Loop 4, and random substitution of at least three amino acids within Loop 4. In embodiments wherein the combinatorial library comprises one or more amino acid modification to the Loop 4 region (alone or in combination with modifications to other regions of the CTLD), the modification(s) can be designed to maintain, modulate, or abrogate the metal ion-binding affinity of the CTLD. Such modifications can affect the plasminogen-binding activity of the CTLD (see, e.g., Nielbo, et al., Biochemistry, 2004, 43 (27), pp 8636-8643; or Graversen 1998).

In further embodiments, the CTLD loop regions can be extended beyond the exemplary constructs detailed in the non-limiting Examples below. Further any combination of the four LSA loops and the LSB loop (Loop 5) in a given library can comprise one or more amino acid modifications (e.g., by insertion, extension, or randomization). Thus, in any of the various embodiments, the modified CTLD can also comprise one or more amino acid modifications to the LSB loop region, either alone or in combination with any one, two, three, or four of the loop regions (Loops 1-4) from the (LSA).

In an aspect, the invention relates to a combinatorial polypeptide library comprising polypeptide members that comprise a modified C-type lectin domain (CTLD), wherein the modified CTLD comprises one or more amino acid modifications in at least one of the four loops in the loop segment A (LSA) of the CTLD, and one or more amino acid modifications in the loop segment B (LSB, or Loop 5), wherein the one or more amino acid modifications comprises randomization of the LSB amino acid residues.

In an embodiment of this aspect, the combinatorial library comprises a modified Loop 3 and a modified Loop 5 region, wherein the modified Loop 3 region comprises randomization of five amino acid residues and the modified Loop 5 region comprises randomization of the three amino acid residues comprising Loop 5. In an embodiment, the combinatorial library comprises a modified Loop 3, a modified Loop 5 region, and a modified Loop 4 region, wherein the modification to Loop 4 abrogates plasminogen binding. In an embodiment, the modification to Loop 4 comprises substitution of lysine 148.

According to the various embodiments described herein, any two, three, four, or five loops from the CTLD region can comprise one or more amino acid modifications (e.g., any random combination of random amino acid modifications to two Loop regions, to three Loop regions, to four Loop regions, or to all five Loop regions). The modified CTLD libraries can further comprise additional amino acid modifications to regions of the CTLD outside of the LSA or LSB regions, such as in the α-helices or β-strands (see, e.g., FIG. 4).

In certain embodiments the recombinant CTLD libraries can be subjected to somatic hypermutation (see, e.g., US Patent Publication 2009/0075378, which is incorporated by reference), DNA shuffling by random fragmentation (Stemmer, PNAS1994), loop shuffling, loop walking, error-prone PCR mutagenesis and other known methods in the art to create sequence diversity in order to generate molecules with optimal binding activity. In further embodiments the recombinant CTLD libraries can optionally retain certain Ca²⁺ coordinating amino acids in the loop regions, and/or plasminogen binding activity can be eliminated (see infra).

In one other embodiment of Strategy 2, a loop region from a CTLD having preferred binding affinity for a target polypeptide (e.g., DR4 and DR5) can be swapped into another CTLD also having preferred binding affinity. For example, a CTLD with preferred binding affinity that has been selected from a library of loop 1 and loop 4 mutants can be further mutated to include mutation(s) in other loops from a CTLD having such mutations where it is has been recognized that the mutations confer preferred binding affinity. Accordingly, information about preferred binding characteristics of CLTDs having mutations in different loops can be used to combine the mutations to produce CTLDs with superior binding affinity.

Strategy 3

A number of peptides with binding activity to the TRAIL receptors have been identified. Crystal structures of the TRAIL ligand in complex with the receptors have identified amino acid sequences involved with the binding interaction (S. G. Hymowitz, et. al., 1999; Sun-Shin Cha et. al., 2000). Furthermore, sequence analyses of peptides and antibodies, which bind the DR5 receptor, have identified a shared tripeptide motif (B. Li et. al., 2006). These peptides can be cloned directly on to either the N or C terminal end trimerization domain as free linear peptides or as disulfide constrained loops using cysteines. Single chain antibodies or domain antibodies capable of binding the TRAIL receptors can also be cloned on to either end of the trimerization domain. Additionally peptides with known binding properties can be cloned directly into any one of the loop regions of the TN CTLD. Peptides selected for as disulfide constrained loops or as complementary determining regions of antibodies might be quite amenable to relocation into the loop regions of the CTLD of human tetranectin. For all of these constructs, binding as a monomer, as well as binding and agonist activation as a trimer, when fused with the trimerization domain can then be tested for.

Strategy 4:

In some case direct cloning of peptides with binding activity may not be enough, further optimization and selection may be required. As example, peptides with known binding to the TRAIL receptors, such as but not limited to those mentioned above, can be grafted into the CTLD of human tetranectin. In order to select for optimal presentation of these peptides for binding, one or more of the flanking amino acids can be randomized, followed by phage display selection for binding. Furthermore, peptides which alone show limited or weak binding can also be grafted into one of the loops of a CTLD library containing randomization of another additional loop, again followed by selection through phage display for increased binding and/or specificity. Additionally, for peptides identified through crystal structures where the specific interacting/binding amino acids are known, randomization of the non binding amino acids can be explored followed by selection through page display for increased binding and receptor specificity. Regions of the TRAIL ligand identified as being responsible for binding can also be examined across species. Conserved amino acids can be retained while randomization and selection for non species conserved positions can be tested.

Methods of Treatment

Another aspect the invention relates to a method of inducing apoptosis in a tumor cell expressing at least one of DR4 and DR5. The method includes contacting the cell with a death receptor agonist of the invention that includes a trimerizing domain and at least one polypeptide that specifically binds to at least one TRAIL death receptor. In one embodiment of this aspect, the method comprises contacting the cell with a trimeric complex of the invention. In various aspects of the invention, proteins and complexes induce caspase-dependent as well as caspase-independent apoptosis.

In another aspect the invention relates to a method of treating a subject having a tumor by administering to the subject a therapeutically effective amount of a death receptor agonist including polypeptide having a trimerizing domain and at least one polypeptide that specifically binds to at least one TRAIL death receptor. In one embodiment of this aspect, the method comprises administering to the subject a trimeric complex of the invention.

Another aspect of the invention is directed to a combination therapy. Formulations comprising death receptor agonists and therapeutic agents are also provided by the present invention. It is believed that such formulations will be particularly suitable for storage as well as for therapeutic administration. The formulations may be prepared by known techniques. For instance, the formulations may be prepared by buffer exchange on a gel filtration column.

The death receptor agonists and therapeutic agents described herein can be employed in a variety of therapeutic applications. Among these applications are methods of treating various cancers. The death receptor agonists and therapeutic agents can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

Effective dosages and schedules for administering the death receptor agonist may be determined empirically, and making such determinations is within the skill in the art. Single or multiple dosages may be employed. It is presently believed that an effective dosage or amount of the death receptor agonist used alone may range from about 1 μg/kg to about 100 mg/kg of body weight or more per day. Interspecies scaling of dosages can be performed in a manner known in the art, e.g., as disclosed in Mordenti et al., Pharmaceut. Res., 8:1351 (1991).

When in vivo administration of the death receptor agonist is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature [see, for example, U.S. Pat. No. 4,657,760; 5,206,344; or 5,225,212]. One of skill will appreciate that different formulations will be effective for different treatment compounds and different disorders, that administration targeting one organ or tissue, for example, may necessitate delivery in a manner different from that to another organ or tissue. Those skilled in the art will understand that the dosage of the death receptor agonist that must be administered will vary depending on, for example, the mammal which will receive the death receptor agonist, the route of administration, and other drugs or therapies being administered to the mammal.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors or any other agent that enhances susceptibility of cancer cells to killing by death receptor agonists which are known in the art.

Preparation and dosing schedules for chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the Apo2L variant, or may be given simultaneously therewith.

The death receptor agonists and therapeutic agents (and one or more other therapies) may be administered concurrently (simultaneously) or sequentially. In particular embodiments, a non natural polypeptide of the invention, or multimeric (e.g., trimeric) complex thereof, and a therapeutic agent are administered concurrently. In another embodiment, a polypeptide or trimeric complex is administered prior to administration of a therapeutic agent. In another embodiment, a therapeutic agent is administered prior to a polypeptide or trimeric complex. Following administration, treated cells in vitro can be analyzed. Where there has been in vivo treatment, a treated mammal can be monitored in various ways well known to the skilled practitioner. For instance, tumor tissues can be examined pathologically to assay for cell death or serum can be analyzed for immune system responses.

Pharmaceutical Compositions

In yet another aspect, the invention relates to a pharmaceutical composition comprising a therapeutically effective amount of the polypeptide of the invention along with a pharmaceutically acceptable carrier or excipient. As used herein, “pharmaceutically acceptable carrier” or “pharmaceutically acceptable excipient” includes any and all solvents, dispersion media, coating, antibacterial and antifungal agents, isotonic and absorption delaying agents, and the like that are physiologically compatible. Examples of pharmaceutically acceptable carriers or excipients include one or more of water, saline, phosphate buffered saline, dextrose, glycerol, ethanol and the like as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the of the antibody or antibody portion also may be included. Optionally, disintegrating agents can be included, such as cross-linked polyvinyl pyrrolidone, agar, alginic acid or a salt thereof, such as sodium alginate and the like. In addition to the excipients, the pharmaceutical composition can include one or more of the following, carrier proteins such as serum albumin, buffers, binding agents, sweeteners and other flavoring agents; coloring agents and polyethylene glycol.

The compositions can be in a variety of forms including, for example, liquid, semi-solid and solid dosage forms, such as liquid solutions (e.g. injectable and infusible solutions), dispersions or suspensions, tablets, pills, powders, liposomes and suppositories. The preferred form will depend on the intended route of administration and therapeutic application. In an embodiment the compositions are in the form of injectable or infusible solutions, such as compositions similar to those used for passive immunization of humans with antibodies. In an embodiment the mode of administration is parenteral (e.g., intravenous, subcutaneous, intraperitoneal, intramuscular). In an embodiment, the polypeptide (or trimeric complex) is administered by intravenous infusion or injection. In another embodiment, the polypeptide or trimeric complex is administered by intramuscular or subcutaneous injection.

Other suitable routes of administration for the pharmaceutical composition include, but are not limited to, rectal, transdermal, vaginal, transmucosal or intestinal administration.

Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. polypeptide or trimeric complex) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An article of manufacture such as a kit containing death receptor agonists and therapeutic agents useful in the treatment of the disorders described herein comprises at least a container and a label. Suitable containers include, for example, bottles, vials, syringes, and test tubes. The containers may be formed from a variety of materials such as glass or plastic. The label on or associated with the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of death receptor agonist and Therapeutic agent.

Therapeutic compositions can be prepared by mixing the desired molecules having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

Production of Polypeptides

The polypeptide of the invention can be expressed in any suitable standard protein expression system by culturing a host transformed with a vector encoding the polypeptide under such conditions that the polypeptide is expressed. Preferably, the expression system is a system from which the desired protein may readily be isolated. As a general matter, prokaryotic expression systems are available since high yields of protein can be obtained and efficient purification and refolding strategies. Thus, selection of appropriate expression systems (including vectors and cell types) is within the knowledge of one skilled in the art. Similarly, once the primary amino acid sequence for the polypeptide of the present invention is chosen, one of ordinary skill in the art can easily design appropriate recombinant DNA constructs which will encode the desired amino acid sequence, taking into consideration such factors as codon biases in the chosen host, the need for secretion signal sequences in the host, the introduction of proteinase cleavage sites within the signal sequence, and the like.

In one embodiment the isolated polynucleotide encodes a polypeptide that specifically binds a TRAIL death receptor and a trimerizing domain. In an embodiment the isolated polynucleotide encodes a first polypeptide that specifically binds a TRAIL death receptor, a second polypeptide that specifically binds a TRAIL death receptor, and a trimerizing domain. In certain embodiments, the polypeptide that specifically binds a TRAIL death receptor (or the first polypeptide and the second polypeptide) and the trimerizing domain are encoded in a single contiguous polynucleotide sequence (a genetic fusion). In other embodiments, polypeptide that specifically binds a TRAIL death receptor (or the first polypeptide and the second polypeptide) and the trimerizing domain are encoded by non-contiguous polynucleotide sequences. Accordingly, in some embodiments the at least one polypeptide that specifically binds a TRAIL death receptor (or the first polypeptide and second polypeptide that specifically bind a TRAIL death receptor) and the trimerizing domain are expressed, isolated, and purified as separate polypeptides and fused together to form the polypeptide of the invention.

These recombinant DNA constructs may be inserted in-frame into any of a number of expression vectors appropriate to the chosen host. In certain embodiments, the expression vector comprises a strong promoter that controls expression of the recombinant polypeptide constructs. When recombinant expression strategies are used to generate the polypeptide of the invention, the resulting polypeptide can be isolated and purified using suitable standard procedures well known in the art, and optionally subjected to further processing such as e.g. lyophilization.

Standard techniques may be used for recombinant DNA molecule, protein, and polypeptide production, as well as for tissue culture and cell transformation. See, e.g., Sambrook, et al. (below) or Current Protocols in Molecular Biology (Ausubel et al., eds., Green Publishers Inc. and Wiley and Sons 1994). Purification techniques are typically performed according to the manufacturer's specifications or as commonly accomplished in the art using conventional procedures such as those set forth in Sambrook et al. (Molecular Cloning: A Laboratory Manual. Cold Spring Harbor Laboratory Press, Cold Spring Harbor, N.Y. (1989), or as described herein. Unless specific definitions are provided, the nomenclature utilized in connection with the laboratory procedures, and techniques relating to molecular biology, biochemistry, analytical chemistry, and pharmaceutical/formulation chemistry described herein are those well known and commonly used in the art. Standard techniques can be used for biochemical syntheses, biochemical analyses, pharmaceutical preparation, formulation, and delivery, and treatment of patients.

It will be appreciated that a flexible molecular linker optionally may be interposed between, and covalently join, the specific binding member and the trimerizing domain. In certain embodiments, the linker is a polypeptide sequence of about 1-20 amino acid residues. The linker may be less than 10 amino acids, most preferably, 5, 4, 3, 2, or 1. It may be in certain cases that 9, 8, 7 or 6 amino acids are suitable. In useful embodiments the linker is essentially non-immunogenic, not prone to proteolytic cleavage and does not comprise amino acid residues which are known to interact with other residues (e.g. cysteine residues).

The description below also relates to methods of producing polypeptides and trimeric complexes that are covalently attached (hereinafter “conjugated”) to one or more chemical groups. Chemical groups suitable for use in such conjugates are preferably not significantly toxic or immunogenic. The chemical group is optionally selected to produce a conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to polypeptides of the invention at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, polypropylene glycol) and polyethylene-polypropylene glycol copolymers, can be employed using the techniques for conjugation described herein for PEG.

The average molecular weight of the PEG employed in the pegylation of the Apo-2L can vary, and typically may range from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are nonhomogeneous preparations sold according to average molecular weight. For example, commercially available PEG(5000) preparations typically contain molecules that vary slightly in molecular weight, usually ±500 D. The polypeptide of the invention can be further modified using techniques known in the art, such as, conjugated to a small molecule compounds (e.g., a chemotherapeutic); conjugated to a signal molecule (e.g., a fluorophore); conjugated to a molecule of a specific binding pair (e.g., biotin/streptavidin, antibody/antigen); or stabilized by glycosylation, PEGylation, or further fusions to a stabilizing domain (e.g., Fc domains).

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, polypropylene glycol) and polyethylene-polypropylene glycol copolymers, can be employed using the techniques for conjugation described herein for PEG. The polyols of the invention include those well known in the art and those publicly available, such as from commercially available sources.

Furthermore, other half-life extending molecules can be attached to the N- or C-terminus of the trimerization domain including serum albumin-binding peptides, IgG-binding peptides or peptides binding to FcRn.

It should be noted that the section headings are used herein for organizational purposes only, and are not to be construed as in any way limiting the subject matter described. All references cited herein are incorporated by reference in their entirety for all purposes.

The Examples that follow are merely illustrative of certain embodiments of the invention, and are not to be taken as limiting the invention, which is defined by the appended claims.

### EXAMPLES

The vectors discussed in the following Examples (pANA) are derived from vectors that have been previously described (See US 2007/0275393. Certain vector sequences are provided in the Sequence Listing and one of skill will be able to derive vectors given the description provided herein. The pPhCPAB phage display vector (SEQ ID NO: 411) is derived from the pCANTAB vector (G.E.) and has the gIII signal peptide coding region fused with a linker to the hTN sequence encoding ALQT (etc.). The C-terminal end of the CTLD region is fused via a linker to the gIII region. Within the CTLD region, nucleotide mutations were generated that did not alter the coding sequence but generated restriction sites suitable for cloning PCR fragments containing altered loop regions. A portion of the loop region was removed between these restriction sites so that all library phage could only express recombinants and not wild-type tetranectin. The vector pCANTAB-TD (SEQ ID NO: 567) contains 36 amino acids starting with Val 17 of human tetranectin and encodes the trimerization domain. The gene III signal peptide is linked to Val 17 and the C-terminus is linked to the E Tag and subsequent gene III phage protein of pCANTAB. The bacterial expression vectors pANA4 (SEQ ID NO: 413), pANA10 (SEQ ID NO: 419) and pANA19 (SEQ ID NO: 565) are all derived from the vector pBAD (Invitrogen), and contain the full-length human tetranectin gene. The vector pANA4 has both the myc and HA Tags at the C-terminus of tetranectin, while pANA10 has the HA and Strep II tags at the C-terminus. The vector pANA19 also has the Strep II and HA tgas but at the N-terminus of human tetranectin. The mammalian expression vectors pANA14 (SEQ ID NO: 564) and pANA20 (SEQ ID NO: 566) are derived from pCEP4 (Invitrogen). The vector pANA14 contains the human tetranectin gene starting at amino acid Vail 7, and has the HA and Strep II tags connected to the C-terminus of the CTLD portion of hTN. The vector pANA20 expresses the full length human tetranectin, and has the Strep II and HA tags at the N-terminus of the protein.

### Example 1

### Library Construction

### Mutation and Extension of Loop 1

The sequence of human tetranectin and the positions of loops 1, 2, 3, 4 (LSA), and 5 (LSB) are shown in FIGS. 1 and 4. For the 1-2 extended libraries of human tetranectin C-type lectin binding domains (“Human 1-2X”), the coding sequences for Loop 1 were modified to encode the sequences shown in Table 2, where the five amino acids AAEGT (SEQ ID NO: 176); human) were substituted with seven random amino acids encoded by the nucleotides NNK NNK NNK NNK NNK NNK NNK (SEQ ID NO: 177); N denotes A, C, G, or T; K denotes G or T. The amino acid arginine immediately following Loop 2 was also fully randomized by using the nucleotides NNK in the coding strand. This amino acid was randomized because the arginine contacts amino acids in Loop 1, and might constrain the configurations attainable by Loop 1 randomization. In addition, the coding sequence for Loop 4 was altered to encode an alanine (A) instead of the lysine (K) in order to abrogate plasminogen binding, which has been shown to be dependent on the Loop 4 lysine (Graversen et al., 1998).

TABLE 2

Amino acids of loop regions from human and mouse tetranectin
(TN).

| | Loop 1 | Loop 2 | Loop 3 | Loop 4 | |
|---|---|---|---|---|---|
| Library | [SEQ ID NO] | [SEQ ID NO] | [SEQ ID NO] | [SEQ ID NO] | Loop 5 |
| Human | DMAAEGTW | DMTGA(R) | NWETEITAQ(P) | DGGKTEN | AAN |
| TN | [178] | [179] | [180] | [181] | |
| Human | DMXXXXXXXW | DMTGA(X) | NWETEITAQ(P) | DGGATEN | AAN |
| 1-2X | [182] | [183] | [180] | [184] | |
| Human | DMXXXXXW | DMXXX(X) | NWETEITAQ(P) | DGGATEN | AAN |
| 1-2 | [185] | [186] | [180] | [184] | |
| Human | XXXXXXXW | DMTGA(R) | NWETEITAQ(P) | DGGXXXXXEN | AAN |
| 1-4 | [187] | [179] | [180] | [188] | |
| Human | DMAAEGTW | DMTGA(R) | NWXXXXXXQ(P) | DGGATEN | AAN |
| 3X 6 | [178] | [179] | [189] | [184] | |
| Human | DMAAEGTW | DMTGA(R) | NWXXXXXXXQ(P) | DGGATEN | AAN |
| 3X 7 | [178] | [179] | [190] | [184] | |
| Human | DMAAEGTW | DMTGA(R) | NWXXXXXXXXQ(P) | DGGATEN | AAN |
| 3X 8 | [178] | [179] | [191] | [184] | |
| Human | DMAAEGTW | DMTGA(R) | NWETXXXXXXAQ(P) | DGGXXXXXXN | AAN |
| 3-4X | [178] | [179] | [192] | [193] | |
| Human | DMAAEGTW | DMTGA(R) | NWEXXXXXX(X) | XGGXXXN | AAN |
| 3-4 | [178] | [179] | [194] | [195] | |
| combo | | | | | |
| Human | DMAAEGTW | DMTGA(R) | NWEXXXXXQ(P) | DGGATEN | XXX |
| 3-5 | [178] | [179] | [196] | [184] | |
| Human 4 | DMAAEGTW | DMTGA(R) | NWETEITAQ(P) | DGGXXXXXXXN | AAN |
| | [178] | [179] | [180] | [197] | |

Parentheses indicate neighboring amino acids not considered part of the loop.
X = any amino acid.

The human Loop 1 extended library was generated using overlap PCR in the following manner (primer sequences are shown in Table 3). Primers 1Xfor (SEQ ID NO: 198) and 1Xrev (SEQ ID NO: 199) were mixed and extended by PCR, and primers BstX1for (SEQ ID NO: 200) and PstBssRevC (SEQ ID NO: 201) were mixed and extended by PCR. The resulting fragments were purified from gels, and mixed and extended by PCR in the presence of the outer primers Bglfor12 (SEQ ID NO: 202 and PstRev (SEQ ID NO: 203). The resulting fragment was gel purified and cut with Bgl II and Pst I and cloned into a phage display vector pPhCPAB or pANA27 (SEQ ID NO: 421). The phage display vector pPhCPAB was derived from pCANTAB (Pharmacia), and contained a portion of the human tetranectin CTLD fused to the M13 gene III protein. The CTLD region was modified to include BglII and PstI restriction enzyme sites flanking Loops 1-4, and the 1-4 region was altered to include stop codons, such that no functional gene III protein could be produced from the vector without ligation of an in-frame insert. pANA27 was derived from pPhCPAB by replacing the BamHI to ClaI regions with the BamHI to ClaI sequence of SEQ ID NO: 421 (pANA27). This replaces the amber suppressible stop codon with a glutamine codon and the vector also includes a gene III truncation.

Ligated material was transformed into electrocompetent XL1-Blue E. coli (Stratagene) and four to eight liters of cells were grown overnight and DNA isolated to generate a master library DNA stock for panning A library size of 1.5×10⁸ was obtained, and clones examined showed diversified sequence in the targeted regions.

TABLE 3

Sequences used in the generation of phage displayed C-type lectin
domain libraries.
M = A or C; N = A, C, G, or T; K = G or T; S = G or C; W = A or T.

| | | SEQ ID |
|---|---|---|
| Name | Sequence | NO |
| 1Xfor | GGCTGGGCCT GAACGACATG NNKNNKNNKN NKNNKNNKNN KTGGGTGGAT | 198 |
| | ATGACTGGCG CC | |
| 1Xrev | GGCGGTGATC TCAGTTTCCC AGTTCTTGTA GGCGATMNNG GCGCCAGTCA | 199 |
| | TATCCACCCA | |
| BstX1for | ACTGGGAAAC TGAGATCACC GCCCAACCTG ATGGCGGCGC AACCGAGAAC | 200 |
| | TGCGCGGTCC TG | |
| PstBssRevC | CCCTGCAGCG CTTGTCGAAC CACTTGCCGT TGGCGGCGCC AGACAGGACC | 201 |
| | GCGCAGTTCT | |
| Bglfor12 | GCCGAGATCT GGCTGGGCCT GAACGACATG | 202 |
| PstRev | ATCCCTGCAG CGCTTGTCGA ACC | 203 |
| Mu1Xfor | GCTGTTCGAA TACGCGCGCC ACAGCGTGGG CAACGATGCG AACATCTGGC | 204 |
| | TGGGCCTCAA CGATATG | |
| Mu1Xrev | GCCGCCGGTC ATGTCGACCC AMNNMNNMNN MNNMNNMNNM NNCATATCGT | 205 |
| | TGAGGCCCAG CCAG | |
| Mu1XSalFor | TGGGTCGACA TGACCGGCGG CNNKCTGGCC TACAAGAACT GGGAGACGGA | 206 |
| | GATCACGACG CAACCCGACG GCGGCGCTGC CGAGAACTG | |
| Mu1XPstRev | CAGCGTTTGT CGAACCACTT GCCGTTGGCT GCGCCAGACA GGGCGGCGCA | 207 |
| | GTTCTCGGCA GCGCCGCCGT CGGGTT | |
| BstBBssH | GCTGTTCGAA TACGCGCGCC ACAGCGTGG | 208 |
| Mu Pst | GGGCAACTGA TCTCTGCAGC GTTTGTCGAA CCACTTGCCG T | 209 |
| 1-2 for | GGCTGGGCCT GAACGACATG NNKNNKNNKN NKNNKTGGGT GGATATGNNK | 210 |
| | NNKNNKNNKA TCGCCTACAA GAACTGGGA | |
| 1-2 rev | GACAGGACGG CGCAGTTCTC GGTTGCGCCG CCATCAGGTT GGGCGGTGAT | 211 |
| | CTCAGTTTCC CAGTTCTTGT AGGCGAT | |
| PstRev12 | ATCCCTGCAG CGCTTGTCGA ACCACTTGCC GTTGGCGGCG CCAGACAGGA | 212 |
| | CGGCGCAGTT CTC | |
| Mu12rev | CGTCTCCCAG TTCTTGTAGG CCAGMNNMNN MNNMNNCATG TCGACCCAMN | 213 |
| | NMNNMNNMNN MNNCATATCG TTGAGGCCCA GCCAG | |
| Mu1234for | GCCTACAAGA ACTGGGAGAC GGAGATCACG ACGCAACCCG ACGGCGGCGC | 214 |
| | TGCCGAGAAC TG | |
| BglBssfor | GAGATCTGGC TGGGCCTCAA CNNSNNSNNS NNSNNSNNSN NSTGGGTGGA | 215 |
| | CATGACTGGC | |
| BssBglrev | TTGCGCGGTG ATCTCAGTCT CCCAGTTCTT GTAGGCGATA CGCGCGCCAG | 216 |
| | TCATGTCCAC CCA | |
| BssPstfor | GACTGAGATC ACCGCGCAAC CCGATGGCGG CNNSNNSNNS NNSNNSGAGA | 217 |
| | ACTGCGCGGT CCTG | |
| PstBssRev | CCCTGCAGCG CTTGTCGAAC CACTTGCCGT TGGCCGCGCC TGACAGGACC | 218 |
| | GCGCAGTTCT | |
| Bglfor | GCCGAGATCT GGCTGGGCCT CA | 219 |
| MuUpsF | GCCATGGCCG CCTTACAGAC TGTGTGCCTG AAG | 220 |
| MuRanR | CGTCTCCCAG TTCTTGTAGG CCAGGAGGCC GCCGGTCATG TCCACCCAMN | 221 |
| | NMNNMNNMNN MNNMNNMNNG TTGAGGCCCA GCCAGAT | |
| MuRanF | GCCTACAAGA ACTGGGAGAC GGAGATCACG ACGCAACCCG ACGGCGGCNN | 222 |
| | KNNKNNKNNK NNKGAGAACT GCGCCGCCCT G | |
| MuDnsR | CGCACCTGCG GCCGCCACAA TGGCAAACTG GCAGATGT | 223 |
| H Loop 1-2-F | ATCTGGCTGG GCCTGAACGA CATGGCCGCC GAGGGCACCT GGGTGGATAT | 224 |
| | GACCGGCGCG CGTATCGCCT ACAAGAAC | |
| H Loop 3-4 | CCGCCATCGG GTTGGGCMNN MNNMNNMNNM NNMNNAGTTT CCCAGTTCTT | 225 |
| Ext R | GTAGGCGATA CG | |
| H Loop 3-4 | GCCCAACCCG ATGGCGGCNN KNNKNNKNNK NNKNNKAACT GCGCCGTCCT | 226 |
| Ext-F | GTCTGGC | |
| H Loop 5-R | CCTGCAGCGC TTGTCGAACC ACTTGCCGTT GGCGGCGCCA GACAGGACGG | 227 |
| | CGCA | |
| M SacII-F | GACATGGCCG CGGAAGGCGC CTGGGTCGAC ATGACCGGCG GCCTGCTGGC | 228 |
| | CTACAAGAAC | |
| M Loop 3-4 | CCGCCGTCGG GTTGGGTMNN MNNMNNMNNM NNMNNGGTCT CCCAGTTCTT | 229 |
| Ext-R | GTAGGCCAGC A | |
| M Loop 3-4 | ACCCAACCCG ACGGCGGCNN KNNKNNKNNK NNKNNKAACT GCGCCGCCCT | 230 |
| Ext-F | GTCTGGC | |
| M Loop 5-R | CTGATCTCTG CAGCGCTTGT CGAACCACTT GCCGTTGGCT GCGCCAGACA | 231 |
| | GGGCGGCGCA GTT | |
| H Loop 3-4 | GCCAGACAGG ACGGCGCAGT TMNNMNNMNN GCCGCCMNNM NNMNNMNNMN | 232 |
| Combo R | NMNNMNNMNN TTCCCAGTTC TTGTAGGCGA TACG | |
| M Loop 3-4 | GCCAGACAGG GCGGCGCAGT TMNNMNNMNN GCCGCCMNNM NNMNNMNNMN | 233 |
| Combo R | NMNNMNNMNN CTCCCAGTTC TTGTAGGCCA GCA | |
| H Loop 3-R | CCGCCATCGG GTTGGGCGGT GATCTCAGTT TCCCAGTTCT TGTAGGCGAT | 234 |
| | ACG | |
| H Loop 4 | GCCCAACCCG ATGGCGGCNN KNNKNNKNNK NNKNNKNNKA ACTGCGCCGT | 235 |
| Ext-F | CCTGTCTGGC | |
| M Loop 3-R | CCGCCGTCGG GTTGGGTGGT GATCTCGGTC TCCCAGTTCT TGTAGGCCAG | 236 |
| | CA | |
| M Loop 4 | ACCCAACCCG ACGGCGGCNN KNNKNNKNNK NNKNNKNNKA ACTGCGCCGC | 237 |
| Ext-F | CCTGTCTGGC | |
| HLoop3F 6 | CTGGCGCGCG TATCGCCTAC AAGAACTGGN NKNNKNNKNN KNNKNNKCAA | 238 |
| | CCCGATGGCG GCGCCACCGA GAAC | |
| HLoop3F 7 | CTGGCGCGCG TATCGCCTAC AAGAACTGGN NKNNKNNKNN KNNKNNKNNK | 239 |
| | CAACCCGATG GCGGCGCCAC CGAGAAC | |
| HLoop3F 8 | CTGGCGCGCG TATCGCCTAC AAGAACTGGN NKNNKNNKNN KNNKNNKNNK | 240 |
| | CAACCCGATG GCGGCGCCAC CGAGAAC | |
| HLoop4R | CCTGCAGCGC TTGTCGAACC ACTTGCCGTT GGCGGCGCCA GACAGGACGG | 241 |
| | CGCAGTTCTC GGTGGCGCCG CCATCGGGTT G | |
| MLoop3F 6 | GTTCTCGGCA GCGCCGCCGT CGGGTTGMNN MNNMNNMNNM NNMNNCCAGT | 242 |
| | TCTTGTAGGC CAGCAGGCCG CCGGTCA | |
| MLoop3F 7 | GTTCTCGGCA GCGCCGCCGT CGGGTTGMNN MNNMNNMNNM NNMNNMNNCC | 243 |
| | AGTTCTTGTA GGCCAGCAGG CCGCCGGTCA | |
| MLoop3F 8 | GTTCTCGGCA GCGCCGCCGT CGGGTTGMNN MNNMNNMNNM NNMNNMNNMN | 244 |
| | NCCAGTTCTT GTAGGCCAGC AGGCCGCCGG TCA | |
| H1-3-4R | GACAGGACCG CGCAGTTCTC GCCSMAGWMC CCSAAGCCGC CMNNGGGTTG | 245 |
| | MNNMNNMNNM NNMNNCTCCC AGTTCTTGTA GGCGATACG | |
| PstLoop4 rev | ATCCCTGCAG CGCTTGTCGA ACCACTTGCC GTTGGCCGCG CCTGACAGGA | 246 |
| | CCGCGCAGTT CTCGCC | |

### Example 2

### Library Construction

### Mutation of Loops 1 and 2

For the Loop 1-2 libraries of human and mouse tetranectin C-type lectin binding domains (“Human 1-2”), the coding sequences for Loop 1 were modified to encode the sequences shown in Table 2, where the five amino acids AAEGT (SEQ ID NO: 176; human) were replaced with five random amino acids encoded by the nucleotides NNK NNK NNK NNK NNK ((SEQ ID NO: 247); N denotes A, C, G, or T; K denotes G or T). In Loop 2 (including the neighboring arginine), the four amino acids TGAR in human were replaced with four random amino acids encoded by the nucleotides NNK NNK NNK NNK (SEQ ID NO: 248). In addition, the coding sequence for Loop 4 was altered to encode an alanine (A) instead of the lysine (K) in the loop, in order to abrogate plasminogen binding, which has been shown to be dependent on the Loop 4 lysine (Graversen et al., 1998).

The human 1-2 library was generated using overlap PCR in the following manner (primer sequences are shown in Table 3). Primers 1-2 for (SEQ ID NO: 210) and 1-2 rev (SEQ ID NO: 211) were mixed and extended by PCR. The resulting fragment was purified from gels, mixed and extended by PCR in the presence of the outer primers Bglfor12 (SEQ ID NO: 202) and PstRev12 (SEQ ID NO: 212). The resulting fragment was gel purified and cut with Bgl II and Pst I and cloned into similarly digested phage display vector pPhCPAB or pANA27, as described above. A library size of 4.86×10⁸ was obtained, and clones examined showed diversified sequence in the targeted regions.

### Example 3

### Library Construction

### Mutation and Extension of Loops 1 and 4

For the Loop 1-4 library of human C-type lectin binding domains (“Human 1-4”), the coding sequences for Loop 1 were modified to encode the sequences shown in Table 2, where the seven amino acids DMAAEGT (SEQ ID NO: 249) were substituted with seven random amino acids encoded by the nucleotides NNS NNS NNS NNS NNS NNS NNS (SEQ ID NO: 250) (N denotes A, C, G, or T; S denotes G or C; K denotes G or T). In addition, the coding sequences for Loop 4 were modified and extended to encode the sequences shown in Table 2, where two amino acids of Loop 4, KT were replaced with five random amino acids encoded by the nucleotides NNS NNS NNS NNS NNS (SEQ ID NO: 251) for human or NNK NNK NNK NNK NNK (SEQ ID NO: 247) for mouse.

The human 1-4 library was generated using overlap PCR in the following manner (primer sequences are shown in Table 3). Primers BglBssfor (SEQ ID NO: 215) and BssBglrev (SEQ ID NO: 216) were mixed and extended by PCR, and primers BssPstfor (SEQ ID NO: 217) and PstBssRev (SEQ ID NO: 218) were mixed and extended by PCR. The resulting fragments were purified from gels, mixed and extended by PCR in the presence of the outer primers Bglfor (SEQ ID NO: 219) and PstRev (SEQ ID NO: 203). The resulting fragment was gel purified and cut with Bgl II and Pst I restriction enzymes, and cloned into similarly digested phage display vector pPhCPAB or pANA27, as described above. A library size of 2×10⁹ was obtained, and 12 clones examined prior to panning showed diversified sequence in the targeted regions.

### Example 4

### Library Construction

### Mutation and Extension of Loops 3 and 4

For the Loop 3-4 extended libraries of human tetranectin C-type lectin binding domains (“Human 3-4X”), the coding sequences for Loop 3 were modified to encode the sequences shown in Table 2, where the three amino acids EIT tetranectin were replaced with six random amino acids encoded by the nucleotides NNK NNK NNK NNK NNK NNK (SEQ ID NO: 252) in the coding strand (N denotes A, C, G, or T; K denotes G or T). In addition, in Loop 4, the three amino acids KTE were replaced with six random amino acids encoded by the nucleotides NNK NNK NNK NNK NNK NNK (SEQ ID NO: 252).

The human 3-4 extended library was generated using overlap PCR in the following manner (primer sequences are shown in Table 3). Primers H Loop 1-2-F (SEQ ID NO: 224) and H Loop 3-4 Ext-R (SEQ ID NO: 225) were mixed and extended by PCR, and primers H Loop 3-4 Ext-F (SEQ ID NO: 226) and H Loop 5-R (SEQ ID NO: 227) were mixed and extended by PCR. The resulting fragments were purified from gels, and mixed and extended by PCR in the presence of additional H Loop 1-2-F (SEQ ID NO: 224) and H Loop 5-R (SEQ ID NO: 227). The resulting fragment was gel purified and cut with Bgl II and Pst I restriction enzymes, and cloned into similarly digested phage display vector pPhCPAB or pANA27, as described above. A library size of 7.9×10⁸ was obtained, and clones examined showed diversified sequence in the targeted regions.

### Example 5

### Library Construction

### Mutation of Loops 3 and 4 and the PRO Between the Loops

For the Loop 3-4 combo library of human tetranectin C-type lectin binding domains (“Human 3-4 combo”), the coding sequences for loops 3 and 4 and the proline between these two loops were altered to encode the sequences shown in Table 2, where the human sequence TEITAQPDGGKTE (SEQ ID NO: 253) were replaced by the 13 amino acid sequence XXXXXXXXGGXXX, (SEQ ID NO: 254) where X represents a random amino acid encoded by the sequence NNK (N denotes A, C, G, or T; K denotes G or T).

The human 3-4 combo library was generated using overlap PCR in the following manner (primer sequences are shown in Table 3). Primers H Loop 1-2-F (SEQ ID NO: 224) and H Loop 3-4 Combo-R (SEQ ID NO: 232) were mixed and extended by PCR and the resulting fragment was purified from gels and mixed and extended by PCR in the presence of additional H Loop 1-2-F (SEQ ID NO: 224) and H loop 5-R (SEQ ID NO: 227). The resulting fragment was gel purified and cut with Bgl II and Pst I restriction enzymes, and cloned into similarly digested phage display vector pPhCPAB or pANA27, as described above. A library size of 4.95×10⁹ was obtained, and clones examined showed diversified sequence in the targeted regions.

### Example 6

### Library Construction

### Mutation and Extension of Loop 4

For the Loop 4 extended libraries of human and mouse tetranectin C-type lectin binding domains (“Human 4”), the coding sequences for Loop 4 were modified to encode the sequences shown in Table 2, where the three amino acids KTE tetranectin were replaced with seven random amino acids encoded by the nucleotides NNK NNK NNK NNK NNK NNK NNK ((SEQ ID NO: 177); N denotes A, C, G, or T; K denotes G or T).

The human 4 extended library was generated using overlap PCR in the following manner (primer sequences are shown in Table 3). Primers H Loop 1-2-F (SEQ ID NO: 224) and H Loop 3-R (SEQ ID NO: 234) were mixed and extended by PCR, and primers H Loop 4 Ext-F (SEQ ID NO: 235) and H Loop 5-R (SEQ ID NO: 227) were mixed and extended by PCR. The resulting fragments were purified from gels, and mixed and extended by PCR in the presence of additional H Loop 1-2-F (SEQ ID NO: 224) and H Loop 5-R (SEQ ID NO: 227). The resulting fragment gel purified and was cut with Bgl II and Pst I restriction enzymes, and cloned into similarly digested phage display vector pPhCPAB or pANA27, as described above. A library size of 2.7×10⁹ was obtained, and clones examined showed diversified sequence in the targeted regions.

### Example 7

### Library Construction

### Mutation with and without Extension of Loop 3

For the Loop 3 altered libraries of human C-type lectin binding domains, the coding sequences for Loop 3 were modified to encode the sequences shown in Table 2, where the six amino acids ETEITA (SEQ ID NO: 255) of mouse tetranectin were replaced with six, seven, or eight random amino acids encoded by the nucleotides NNK NNK NNK NNK NNK NNK (SEQ ID NO: 252), NNK NNK NNK NNK NNK NNK NNK (SEQ ID NO: 177), and NNK NNK NNK NNK NNK NNK NNK NNK (SEQ ID NO: 256); N denotes A, C, G, or T; and K denotes G or T. In addition, in Loop 4, the three amino acids KTE in human were replaced with six random amino acids encoded by the nucleotides NNK NNK NNK NNK NNK NNK (SEQ ID NO: 252). In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

The human Loop 3 altered library was generated using overlap PCR in the following manner. Primers HLoop3F6, HLoop3F7, and HLoop3F8 (SEQ ID NOS: 238-240, respectively) were individually mixed with HLoop4R (SEQ ID NO: 241) and extended by PCR. The resulting fragments were purified from gels, and mixed and extended by PCR in the presence of oligos H Loop 1-2F (SEQ ID NO: 224), HuBglfor (GCC GAG ATC TGG CTG GGC CTG A (SEQ ID NO: 257)) and PstRev (SEQ ID NO: 203). The resulting fragments were gel purified, digested with BglI and PstI restriction enzymes, and cloned into similarly digested phage display vector pPhCPAB or pANA27, as above. After library generation, the three libraries were pooled for panning.

### Example 8

### Mutation of Loops 3 and 5

For the loop 3 and 5 altered libraries of human tetranectin C-type lectin binding domains, the coding sequences for loops 3 and 5 were modified to encode the sequences shown in Table 2, where the five amino acids TEITA (SEQ ID NO: 258) of human tetranectin were replaced with five amino acids encoded by the nucleotides NNK NNK NNK NNK NNK (SEQ ID NO: 247), and the three amino acids AAN of human were replaced with three amino acids encoded by the nucleotides NNK NNK NNK. In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

The human loop 3 and 5 altered library was generated using overlap PCR in the following manner. Primers h3-5AF (SEQ ID NO: 422) and h3-5AR (SEQ ID NO: 423) were mixed and extended by PCR, and primers h3-5BF (SEQ ID NO: 424) and h3-5 BR (SEQ ID NO: 425) were mixed and extended by PCR. The resulting fragments were purified from gels, and mixed and extended by PCR in the presence of h3-50F (SEQ ID NO: 426) and PstRev (SEQ ID NO: 203). The resulting fragment was gel purified, digested with Bgl I and Pst I restriction enzymes, and cloned into similarly digested phage display vector pPhCPAB or pANA27 as above.

### Example 9

### Construction of Libraries and Clones for Selection and Screening of Agonists for TRAIL Receptors DR4 and DR5

Phage libraries expressing linear or cyclized randomized peptides of varying lengths can be purchased commercially from manufacturers such as New England Biolabs (NEB). Alternatively, phage display libraries containing randomized peptides in loops of the C-type lectin domain (CTLD) (SEQ ID NO: 117) of human tetranectin can be generated. Loops 1, 2, 3, and 4 are shown in FIG. 4. Amino acids within these loops can be randomized using an NNS or NNK overlapping PCR mutagenesis strategy. From one to seven codons in any one loop may be replaced by a mutagenic NNS or NNK codon to generate libraries for screening; alternatively, the number of mutagenized amino acids may exceed the number being replaced (two amino acids may be replaced by five, for example, to make larger randomized loops). In addition, more than one loop may be altered at the same time. The overlap PCR strategy can generate either a Kpn I site in the final DNA construct between loops 2 and 3, which alters one of the amino acids between the loops, exchanging a threonine for the original alanine. Alternatively, a BssH II site can be incorporated between loops 2 and 3 that does not alter the original amino acid sequence.

### Example 10

### Selection and Screening of Agonists for TRAIL Receptors DR4 and DR5

Bacterial colonies expressing phage are generated by infection or transfection of bacteria such as E. coli TG-1 or XL-1 Blue using either glycerol phage stocks of phage libraries or library DNA, respectively. Fifty milliliters of infected/transfected bacteria at an O.D.₆₀₀ of 1.0 are grown for 15 min at room temperature (RT), after which time 40% of the final concentration of selectable drug marker is added to the culture and incubated for 1 h at 37° C. Following that incubation the remaining drug for selection is added and incubated for another hour at 37° C. Helper phage VCS M13 are added and incubated for 2 h. Kanamycin (70 μg/mL) is added to the culture, which is then incubated overnight at 37° C. with shaking Phage are harvested by centrifugation followed by cold precipitation of phage from supernatant with one third volume of 20% polyethylene glycol (PEG) 8000/2.5 M NaCl. Phage are resuspended in a buffer containing a protease inhibitor cocktail (Roche Complete Mini EDTA-free) and are subsequently sterile filtered. Phage libraries are titered in E. coli TG-1, XL1-Blue, or other appropriate bacterial host.

Phage are panned in rounds of positive selection against human DR4 and/or DR5. Human DR4 and DR5 (aka human TRAIL death receptors 1 and 2) are commercially available in a soluble form (Antigenix America, Cell Sciences, or as Fc (Genway Biotech, R&D Systems) or GST fusions (Novus Biologicals). Soluble DR4 or DR5 in PBS is bound directly to a solid support, such as the bottom of a microplate well (Immulon 2B plates) or to magnetic beads such as Dynabeads. About 250 ng to 500 ng of soluble DR4 or DR5 is bound to the solid substrate by incubation overnight in PBS at either 4° C. or RT. The plates (or beads) are then washed three times in PBS/0.05% Tween 20, followed by addition of a blocking agent such as 1% BSA, 0.05% sodium azide in PBS and is incubated for at least 0.5 h at RT to prevent binding of material in future steps to non-specific surfaces. Blocking agents such as PBS with 3% non-fat dry milk or boiled casein can also be used.

In an alternative protocol, in order to bind DR4 or DR5Fc fusion proteins, plates or beads are first incubated with 0.5-1 μg of a commercially available anti-Fc antibody in PBS. The plates (or beads) are washed and blocked with 1% BSA, 0.05% sodium azide in PBS as above, and are then incubated with death receptor fusion protein at 5 μg/mL and incubated for 2 h at RT. Plates are then washed three times with PBS/0.05% Tween 20.

Phage libraries at a concentration of about 10¹¹ or 10¹² pfu/mL are added to the wells (or beads) containing directly or indirectly bound death receptor. Phage are incubated for at least 2 h at RT, although to screen for different binding properties the incubation time and temperature can be varied. Wells are washed at least eight times with PBS/0.05% Tween 20, followed by PBS washes (8×). Wells can be washed in later rounds of selection with increasingly acidic buffers, such as 100 mM Tris pH 5.0, Tris pH 4.0, and Tris pH 3.0. Bound phages are eluted by trypsin digestion (100 μL of 1 mg/mL trypsin in PBS for 30 min). Bound phages can also be eluted using 0.1 M glycine, pH 2.2. Alternatively, bound phages can be eluted using TRAIL (available commercially from AbD Serotec) to select for CTLDs or peptides that compete with TRAIL for binding to the death receptors. Further, bound phage can be eluted with compounds that are known to compete with TRAIL for death receptor binding.

Eluted phage are incubated for 15 min with 10 mL of freshly grown bacteria at an OD₆₀₀ of 0.8, and the infected bacteria are treated as above to generate phage for the second round of panning Two or three additional rounds of positive panning are performed.

As an alternative to using DR4 and/or DR5 directly or indirectly bound to a support, DR4 and/or DR5 expressed endogenously by cancer cell lines or expressed by transfected cells such as 293 cells may be used in rounds of positive selection. For transfected cells, transfection is performed two days prior to panning using the Qiagen Attractene™ protocol, for example, and an appropriate expression plasmid such as pcDNA3.1, pCEP4, or pCEP5 bearing DR4 or DR5. Cells are dissociated in a non-trypsin dissociation buffer and 6×10⁶ cells are resuspended in 2 mL IMDM buffer. Phage to be panned are dialyzed prior to being added to cells and incubated for 2 h, RT. Cells are washed by pelleting and resuspending multiple times in IMDM, and phage are eluted with glycine buffer.

In order to select those peptides that have affinity for DR4 and/or DR5 but not decoy receptors, negative selection rounds or negative selection concomitant with positive selection are performed. Negative selection is done using the decoy receptors DcR1, DcR2, soluble DcR3, and/or osteoprotegerin (OPG, R&D systems). OPG and soluble DcR3 are commercially available(GeneTex, R&D systems), as are DcR1 and DcR2 conjugated to Fcor GST (R&D Systems, Novus Biologicals). For negative selection rounds, decoy receptor is bound to plates or beads and blocked as described above for positive rounds of selection. Beads are more desirable as a larger surface area of negative selection molecules can be exposed to the library being panned. The primary library or the phage from other rounds of positive selection are incubated with the decoy receptors for 2 h at room temperature, or overnight at 4° C. Unbound phage are then removed and subjected to a positive round of selection.

Positive selection is also performed simultaneously with negative selection. Wells or beads coated with soluble DR4 or DR5 are blocked and exposed to the primary library or phage from a selection round as described above, but a decoy receptor such as DcR1 is included at a concentration of 10 μg/mL. Incubation time may be extended from 2 h to several days at 4° C. prior to elution in this strategy in order to obtain phage with greater specificity and affinity for DR4 or DR5. Negative selection using DR4, in order to obtain DR5-specific, or DR5, in order to obtain DR4-specific binders, can also be performed using the approaches detailed above. Negative selection can also be performed on cancerous or transfected cells that express one or more of the decoy receptors.

Negative selection is performed similarly to positive selection as described above except that phage are recovered from the supernatant after spinning cells down after incubation and then used in a positive round of selection.

### Example 11

### Panning of Human Library 1-4 on Human DR4 and DR5

Phage generated from human library 1-4 were panned on recombinant TRAIL R1 (DR4)/Fc chimera, and TRAIL R2 (DR5)/Fc chimera. Screening of these binding panels after three, four, and/or five rounds of panning using an ELISA plate assay identified receptor-specific binders in all cases.

1. Panning on DR4 Receptor

Panning was performed using the human Loop1-4 library of human CTLDs on DR4/Fc antigen-coated (R&D Systems) wells prepared fresh the night before bound with 250 ng to 1 μg of the carrier free target antigen diluted in 100 μL of PBS per well. Antigen plates were incubated overnight at 4° C. then for 1 hour at 37° C., washed twice with PBS/0.05% Tween 20 and twice with PBS, and then blocked with 1% BSA/PBS for 1 hr at 37° C. prior to panning Six wells were used in each round, and phage were bound to wells for two hours at 37° C. using undiluted, 1:10, and 1:100 dilutions in duplicates of the purified phage supernatant stock. Since target antigens were expressed as Fc fusion proteins, phage supernatant stocks contained 1 μg/mL soluble IgG1 Fc acting as soluble competitor. In addition, prior to target antigen binding, phage supernatants were pre-bound to antigen wells with human IgG1 Fc to remove Fc binders (no soluble IgG1 Fc competitor was present during the pre-binding).

To produce phage for the initial round of panning, 10 μg of library DNA was transformed into electrocompetent TG-1 bacteria and grown in a 100 mL culture containing SB with 40 μg/ml carbenicillin and 2% glucose for 1 hour at 37° C. The carbenicillin concentration was then increased to 50 μg/ml and the culture was grown for an additional hour. The culture volume was then increased to 500 mL, and the culture was infected with helper phage at a multiplicity of infection (MOI) of 5×10⁹ pfu/mL and grown for an additional hour at 37° C. The bacteria were spun down and resuspended in 500 mL SB containing 50 μg/ml carbenicillin and 100 μg/ml kanamycin and grown overnight at room temperature shaking at 250 rpm. The following day bacteria were spun out and the phage precipitated with a final concentration of 4% PEG/0.5 M NaCl on ice for 1 hr. Precipitated phage were then spun down at 10,500 rpm for 20 minutes at 4° C. Phage pellets were resuspended in 1% BSA/PBS containing the Roche EDTA free complete protease inhibitors. Resuspended phage were then spun in a microfuge for 10 minutes at 13,200 rpm and passed through a 0.2 μM filter to remove residual bacteria.

50 μL of the purified phage supernatant stock per well were pre bound to the IgG Fc coated wells for 1 hr at 37° C. and then transferred to the target antigen coated well at the appropriate dilution for 2 hrs at 37° C. as described above. Wells were then washed with PBS/0.05% Tween 20 for 5 minutes pipeting up and down (1 wash at round 1, 5 washes at round 2, and 10 washes at rounds 3 and 4). Target antigen bound phage were eluted with 60 μL per well acid elution buffer (glycine pH 2) and then neutralized with 2M Tris 3.6 μL/well. Eluted phage were then used to infect TG-1 bacteria (2 mL at OD₆₀₀ of 0.8-1.0) for 15 minutes at room temperature. The culture volume was brought up to 10 mL in SB with 40 μg/ml carbenicillin and 2% glucose and grown for 1 hour at 37° C. shaking at 250 rpm. The carbenicillin concentration was then increased to 50 μg/ml and the culture was grown for an additional hour. The culture volume was then increased to 100 mL, and the culture was infected with helper phage at an MOI of 5×10⁹ pfu/mL and grown for an additional hour at 37° C. The bacteria were spun down and resuspended in 100 mL SB containing 50 μg/ml carbenicillin and 100 μg/ml kanamycin and grown overnight at room temperature with shaking at 250 rpm. Subsequent rounds of panning were performed similarly adjusting for smaller culture volumes, and with increased washing in later rounds. Clones were panned on DR4/Fc for four rounds and clones obtained from screening rounds three and four.

2. Phage ELISA

Panning was performed using the TG-1 strain of bacteria for at least four rounds. At each round of panning sample titers were taken and plated on LB plates containing 50 μg/mL carbenicillin and 2% glucose. To screen for specific binding of phagemid clones to the receptor target, individual colonies were picked from these titer plates from the later rounds of panning and grown up overnight at room temperature with shaking at 250 rpm in 250 μL of 2xYT medium containing 2% glucose and 50 μg/mL carbenicillin in a polypropylene 96-well plate with an air-permeable membrane on top. The following day a replica plate was set up in a 96-deep-well plate by inoculating 500 μL of 2xYT containing 2% glucose and 50 μg/mL carbenicillin with 30 μL of the previous overnight culture. The remaining overnight culture was used to make a master stock plate by adding 100 μL of 50% glycerol to each well and storing at −80° C. The replica culture plate was grown at 37° C. with shaking at 250 rpm for approximately 2 hrs until the OD₆₀₀ was 0.5-0.7. The wells were then infected with K07 helper phage to 5×10⁹ pfu/mL mixed and incubated at 37° C. for 30 minutes without shaking, then incubated an addition 30 minutes at 37° C. with shaking at 250 rpm. The cultures were then spun down at 2500 rpm and 4° C. for 20 minutes. The supernatants were removed from the wells and the bacterial cell pellets were re-suspended in 500 μL of 2xYT containing 50 μg/mL carbenicillin and 50 μg/mL kanamycin. An air-permeable membrane was placed on the culture block and cells were grown overnight at room temperature with shaking at 250 rpm.

On day 3, cultures were spun down and supernatants containing the phage were blocked with 3% milk/PBS for 1 hr at room temperature. An initial Phage ELISA was performed using 75-100 ng of antigen bound per well. Non-specific binding was measured using 75-100 ng of human IgG1 Fc per well. DR4/Fc antigen (R&D Systems)-coated wells and IgG Fc coated wells were prepared fresh the night before by binding the above amount of antigen diluted in 100 μL of PBS per well. Antigen plates were incubated overnight at 4° C. then for 1 hour at 37° C., washed twice with PBS/0.05% Tween 20 and twice with PBS, and then blocked with 3% milk/PBS for 1 hr at 37° C. prior to the ELISA. Blocked phage were bound to blocked antigen-bound plates for 1 hr then washed twice with 0.05% Tween 20/PBS and then twice more with PBS. A HRP-conjugated anti-M13 secondary antibody diluted in 3% milk/PBS was then applied, with binding for 1 hr and washing as described above. The ELISA signal was developed using 90 μL TMB substrate mix and then stopped with 90 μL 0.2 M sulfuric acid, then ELISA plates were read at 450 nM. Secondary ELISA screens were performed on the positive binding clones identified, screening against additional TRAIL receptors and decoy receptors to test for specificity (DR4, DR5, DcR1 and DcR2). Secondary ELISA screens were performed similarly to the protocol detailed above.

DR4 specific binding clones. Examples of amino acid sequences for Loops 1 and 4 selected for specific binding to the DR4 receptor from the human TN 1-4 library are detailed below in Table 4.

TABLE 4
Sequences of Loops 1 and 4 from binders to
human DR4

| | | Loop 1 | | Loop 4 |
|---|---|---|---|---|
| | Loop 1 | SEQ ID | Loop 4 | SEQ ID |
| Clones | Sequence | NO | Sequence | NO |
| 014-42.3D11 | GWLEGAGW | 259 | DGGWHWRWEN | 260 |
| 014-42.3B8 | GWLEGVGW | 261 | DGGEHWGWEN | 262 |
| 014-42.3D9 | GYLAGVGW | 263 | DGGRGFRWEN | 264 |
| 014-42.3C7 | GWLEGYGW | 265 | DGGTWWEWEN | 266 |
| 014-42.3D10 | GYLEGYGW | 267 | DGGATIAWEN | 268 |
| 014-42.3G8 | GWLqGVGW | 269 | DGGRGWPWEN | 270 |
| 014-40.3E11 | GYLAGYGW | 271 | DGGPSIWREN | 272 |
| 014-40.3B2 | GYIEGTGW | 273 | DGGSNWAWEN | 274 |
| 014-40.3B3 | GYMSGYGW | 275 | DGGMMARWEN | 276 |
| 014-40.3A3 | GFMVGRGW | 277 | DGGSMWPWEN | 278 |
| 014-40.3H2 | MVTRPPYW | 279 | DGGWVMSFEN | 280 |
| 014-40.3E9 | PFRVPqWW | 281 | DGGYGPVqEN | 282 |
| 064-40.2G11 | GWLEGAGW | 259 | DGGWQWRWEN | 283 |
| 064-40.2E10 | GYLDGVGW | 284 | DGGQGCRWEN | 285 |
| 064-36.1E4 | VLRLAWSW | 286 | DGGKRNGCEN | 287 |
| 064-40.1E11 | WLSLFSPW | 288 | DGGRGVRGEN | 289 |
| 064-36.1B7 | GWMAGVGW | 290 | DGGRRLPWEN | 291 |
| 064-40.2C7 | SYRLHYGW | 292 | DGGRRWLGEN | 293 |
| 064-36.1E1 | IWPLRFRW | 294 | DGGFVTRKEN | 295 |
| 064-40.2D9 | WqLYYRYW | 296 | DGGVGCMVEN | 297 |
| 064-36.1G4 | RCLqGVGW | 298 | DGGRGWPWEN | 270 |
| 064-36.1E12 | GCTqGQGW | 299 | DGGKKWKWEN | 300 |
| 064-21.1A5 | GFLqGNGW | 301 | DGGMWDRWEN | 302 |
| 064-40.2A10 | GVLqRGGW | 303 | DGGPGGEREN | 304 |
| 064-40.2C3 | PFRVLqQWW | 305 | DGGCGPVqQEN | 306 |
| 064-40.2D2 | PFRGPqQWW | 307 | DGGYGPVGEN | 308 |
| 064-40.2E5 | ARFAMWqQW | 309 | DGGRAGVGEN | 310 |
| 064-40.2C4 | GWLQGYGW | 311 | DGGqQIGWGEN | 312 |
| 064-40.2C5 | AWRSWLNW | 313 | DGGREqQRREN | 314 |
| 029-61.1E11 | GWLEGVGW | 261 | DGGWPFSNEN | 315 |
| 029-61.1A5 | GWLMGTGW | 316 | DGGWWNRWEN | 317 |
| 029-62.2C5 | VRRMGFHW | 318 | DGGRVAVGEN | 319 |
| 029-62.2B3 | RYHVQALW | 320 | DGGRVRPREN | 321 |
| 029-62.4F5 | IqCSPPLW | 322 | DGGAVqqQEN | 323 |
| 029-62.7D10 | GLARQqGW | 324 | DGGKGRPREN | 325 |
| 064-40.1G9 | GWLSGVGW | 326 | DGGWAHAWEN | 327 |
| 064-40.1C7 | GWLEGVGW | 261 | DGGGGVRWEN | 328 |
| 064-98.1G6 | GWLSGYGW | 329 | DGGRVWSWEN | 330 |
| 064-99.2H5 | GLLSDWWW | 331 | DGGGNqSREN | 332 |
| 064-101.4B10 | QWVAFWSW | 333 | DGGSAVSGEN | 334 |
| 064-101.4H1 | PYTSWGLW | 335 | DGGVGGRGEN | 336 |
| 064-40.1G11 | VARWLLKW | 337 | DGGMCKPCEN | 338 |
| 064-36.1E10 | GFLAGVGW | 339 | DGGWWTRWEN | 340 |
| 064-36.1G10 | GYLQGSGW | 341 | DGGWKTRWEN | 342 |
| 064-36.1D7 | VRHWLqLW | 343 | DGGGWWKGEN | 344 |

3. Panning on DR5 receptor

Panning on the DR5 receptors was performed similarly to that detailed above for the DR4 receptor with the exception that five rounds of panning were performed and pre-binding was performed on wells coated with BSA rather than IgG1 Fc. However phage supernatant stocks contained soluble IgG1 Fc to act as soluble competitor for Fc binding during each round. DR5-specific binding clones were obtained screening from round 5. Amino acid sequences for Loops 1 and 4 obtained from the clones for DR5 specific binding are shown below in Table 5, below.

TABLE 5
Sequences of Loops 1 and 4 from binders to
human DR5

| | | Loop 1 | | Loop 4 |
|---|---|---|---|---|
| | Loop 1 | SEQ | Loop 4 | SEQ ID |
| Clone | Sequence | ID NO | Sequence | NO |
| 029-15.A3C | RATLRPRW | 345 | DGG----KN | 346 |
| 029-15.A7D | RAMLRSRW | 347 | DGGRWFQGKN | 348 |
| 029-15.A5A | RALFRPRW | 349 | DGGPWYLKEN | 350 |
| 029-15.A1H | RAVLRPRW | 351 | DGGWVLGGKN | 352 |
| 029-15.A8G | RAWLRPRW | 353 | DGGTLVSGEN | 354 |
| 029-15.B10A | RVIRRSMW | 355 | DGGQKWMAEN | 356 |
| 029-15.B2H | RVLQRPVW | 357 | DGGMVWSMEN | 358 |
| 029-15.B12H | RVqLRPRW | 359 | EGGFRRHAKN | 360 |
| 029-15.A6C | RVVRLSEW | 361 | DGGMLWAMEN | 362 |
| 029-15.B3G | RVISAPVW | 363 | DGGQQWAMEN | 364 |
| 029-15.B12G | RVLRRPQW | 365 | NGGDWRIPEN | 366 |
| 029-15.A6B | RVMMRPRW | 367 | DGGMWGAMEN | 368 |
| 029-15.B4F | RVMRRVLW | 369 | DGGRRETMKN | 370 |
| 029-15.A9G | RVMRRPLW | 371 | DGGRGQQWEN | 372 |
| 029-15.B11F | RVMRRREW | 373 | DGAQLMALEN | 374 |
| 029-15.B11C | RVWRRSLW | 375 | DGGHLVKQKN | 376 |
| 029-15.A4G | KRRWYGGW | 377 | DGGVNTVREN | 378 |
| 029-15.B9F | KRVWYRGW | 379 | DGGMRRRREN | 380 |
| 029-15.A9B | AVIRRPLW | 381 | DGGMKYTMEN | 382 |
| 029-15.B4H | ELVTSRLW | 383 | DGGVMqLGEN | 384 |
| 029-15.B11G | ELGTSRLW | 385 | DGGVMqLGEN | 384 |
| 029-15.B3A | FRGWLRWW | 386 | DDGARVLAEN | 387 |
| 029-15.B1A | GRLKGIGW | 388 | DGGRPQWGEN | 389 |
| 029-15.A4E | GVWqSFPW | 390 | DGGLGYLREN | 391 |
| 029-15.B3E | HLVSLAPW | 392 | DGGGMHQGKN | 393 |
| 029-15.A11H | HIFIDWGW | 394 | DGGVMTMGEN | 395 |
| 029-15.B4D | PVMRGVTW | 396 | DGGRSWVWEN | 397 |
| 029-15.A2E | QLVTVGPW | 398 | DGGVMHRTEN | 399 |
| 029-15.A7F | QLVVqMGW | 400 | DGGWMTVGEN | 401 |
| 029-15.B11A | VAIRRSVW | 402 | DGGERAHSEN | 403 |
| 029-15.B2B | WVMRRPLW | 404 | DGGSMGWREN | 405 |
| 029-15.A8E | WRSMVVWW | 406 | DGGKHTLGEN | 407 |
| 029-15.B3D | ELRTDGLW | 408 | DGGVMRRSEN | 409 |

As stated above, Loop 1 contained seven randomized amino acids in the screened library, whereas Loop 4 had an insertion of 5 randomized amino acids in place of 2 native amino acids (underlined regions in Table 5). In some clones having a glutamine (Q) in an altered loop, an amber-suppressible stop codon (TAG) encoded the glutamine, and this is indicated by a lower case “q”. During panning, a few clones containing changes outside of these regions were identified, for example, in Loop 4, the carboxy-flanking amino acid has been altered from E to K in several instances.

### Example 12

### Subcloning and Production of ATRIMER™ Binders to Human DR4 and DR5 receptors

The loop region DNA fragments were released from DR4/DR5 binder DNA by double digestion with BglII and MfeI restriction enzymes, and were ligated to bacterial expression vectors pANA4, pANA10 or pANA19 to produce secreted ATRIMERS™ in E. coli.

The expression constructs were transformed into E. coli strains BL21 (DE3), and the bacteria were plated on LB agar with ampicillin. Single colony on a fresh plate was inoculated into 2xYT medium with ampicillin. The cultures were incubated at 37° C. in a shaker at 200 rpm until OD600 reached 0.5, then cooled to room temperature. Arabinosis was added to a final concentration of 0.002-0.02%. The induction was performed overnight at room temperature with shaking at 120-150 rpm, after which the bacteria were collected by centrifugation. The periplasmic proteins were extracted by osmotic shock or gentle sonication.

The 6xHis-tagged ATRIMERS™ were purified by Ni⁺-NTA affinity chromatography. Briefly, periplasmic proteins were reconstituted in a His-binding buffer (100 mM HEPES, pH 8.0, 500 mM NaCl, 10 mM imidazole) and loaded onto a Ni⁺-NTA column pre-equivalent with His-binding buffer. The column was washed with 10× vol. of binding buffer. The proteins were eluted with an elution buffer (100 mM HEPES, pH 8.0, 500 mM NaCl, 500 mM imidazole). The purified proteins were dialyzed into PBS buffer and bacterial endotoxin was removed by anion exchange.

The strep II-tagged ATRIMERS™ were purified by Strep-Tactin affinity chromatography. Briefly, periplasmic proteins were reconstituted in 1× binding buffer (20 mM Tris-HCl, pH 8.5, 150 mM NaCl, 2 mM CaCl₂, 0.1% Triton X-100) and loaded onto a Strep-Tactin column pre-equivalent with binding buffer. The column was washed with 10× vol. of binding buffer. The proteins were eluted with an elution buffer (binding buffer with 2.5 mM desthiobiotin). The purified proteins were dialyzed into binding buffer and bacterial endotoxin was removed by anion exchange.

### Example 13

### Characterization of the Affinity of Human DR4 and DR5 Receptor Binders Using Biacore

Apparent affinities of the trimeric DR4 and DR5 binders are provided in Tables 6 and 7, respectively. Immobilization of an anti-human IgG Fc antibody (Biacore) to the CM5 chip (Biacore) was performed using standard amine coupling chemistry and this surface was used to capture recombinant human DR4 or DR5 receptor Fc fusion protein (R&D Systems). ATRIMER™ dilutions (1-500 nM) were injected over the DR4 and DR5 receptor surface at 30 μl/min and kinetic constants were derived from the sensorgram data using the Biaevaluation software (version 3.1, Biacore). Data collection was 3 minutes for the association and 5 minutes for dissociation. The anti-human IgG surface was regenerated with a 30 s pulse of 3 M magnesium chloride. All sensorgrams were double-referenced against an activated and blocked flow-cell as well as buffer injections.

TABLE 6
Apparent affinities of DR4 receptor
binders from H Loop 1-4 library.

| Analyte | Kₐ (1/M · s) | K_{d} (1/s) | K_{A} (1/M) | K_{D} (nM) |
|---|---|---|---|---|
| 014-42.3D10 | 1.22E+04 | 1.85E−03 | 6.58E+06 | 152 |
| 014-42.3B8 | 1.12E+05 | 1.01E−03 | 1.11E+08 | 9.01 |
| 014-42.3D11 | 1.33E+04 | 5.26E−04 | 2.53E+07 | 39.5 |

TABLE 7
Apparent affinities of DR5 receptor
binders from H Loop 1-4 library.

| Analyte | Kₐ (1/M · s) | K_{d} (1/s) | K_{A} (1/M) | K_{D} (nM) |
|---|---|---|---|---|
| 1a7b (=A8G) | 4.05E+04 | 6.29E−04 | 6.43E+07 | 15.6 |
| 8b6b (=A1H) | 1.29E+04 | 5.06E−04 | 2.56E+07 | 39.1 |
| 9b3d (=B3D) | 116 | 1.04E−04 | 1.11E+06 | 899 |
| 2a1a (=B9F) | 4.38E+04 | 1.84E−03 | 2.38E+07 | 42.8 |
| 4a8c (=A3C) | 6.30E+04 | 3.62E−04 | 1.74E+08 | 5.74 |

Description of Cell Assay.

H2122 lung adenocarnoma cells (ATCC# CRL-5985) and A2780 ovarian carcinoma cells (European Collection of Cell Culture, #93112519) were incubated at 1×10⁴ cells/well with DR5 ATRIMERS™ (20 μg/mL) or TRAIL (0.2 μg/mL, R&D Systems) in 10% FBS/RMPI media (Invitrogen) in a 96-well white opaque plate (Costar). The control wells received media and the respective buffer: TBS for DR5 ATRIMERS™ and PBS for TRAIL. After 20 hours, cell viability was determined by ViaLight Plus (Lonza) and detected on a Glomax luminometer (Promega). Data were expressed as percent cell death relative to the respective buffer control. The mean and standard error of triplicates were plotted using Excel. Five DR5 ATRIMERS™ were tested: 4a8c, 2a1a, 1a7b, 9b3d and 8b6b. Three DR5 ATRIMERS™ (4a8c, 1a7b and 8b6b) showed over 50% killing in both cell lines. Similar data were obtained in a separate experiment.

### Example 14

### Panning and Selection of Additional DR4 Specific Clones with More Stringent Binding and Washing Conditions

ATRIMER™ 29p61P1 E11 (referred to as 029-61.1E11 in Table 4) demonstrated killing activity on the Burkitt's lymphoma cell line ST486 with an ED50 of 217 nM. In order to obtain additional DR4-binding clones with better agonist activity, the human loop 1-4 library (see Example 3) was re-panned on DR4 using more stringent binding and washing conditions. Panning was performed as detailed in Example 10 with the exception that prior to panning the precipitated phage were re-suspended in Buffer D (0.5% Boiled Casein in TBS, pH 7.4, 0.025% Tween 20, 2 mM CaCl₂). Phage were pre-bound to wells coated with IgG Fc and blocked in buffer D. Binding to DR4/Fc was performed in Buffer D for 2 hrs at room temperature. Washes were also performed using buffer D, and bound phage were eluted using 0.1M glycine pH 2.2. Clones obtained from rounds 4 and 5 were screened by ELISA for specific binding as previously detailed. Clones which bound specifically to DR4 were sequenced, and in addition to the previously obtained clones detailed in Table 4, additional novel sequences were obtained (Table 8).

TABLE 8
Sequences of loops 1 and 4 for new binders to
human DR4

| | | Loop 1 | | Loop 4 |
|---|---|---|---|---|
| | | SEQ | | SEQ |
| Clone | Loop 1 | ID NO | Loop 4 | ID NO |
| 71p88P1B3 | GWLEGSGW | 428 | DGGVQWRWEN | 436 |
| 71p88P1G4 | GYMTGVGW | 429 | DGGRSWKWEN | 437 |
| 71p88P1G1 | GWMEGVGW | 430 | DGGPPWRWEN | 438 |
| 71p88P1F2 | GWLEGSGW | 428 | DGGFPARWEN | 439 |
| 71p88P1A1 | GWMDGSGW | 431 | DGGRLWRWEN | 440 |
| 71p88P1G11 | GWMAGVGW | 290 | DGGPGLRWEN | 441 |
| 71p88P1A3 | GYLAGTGW | 432 | DGGRVLAWEN | 442 |
| 71p88P1B9 | GWLAGSGW | 433 | DGGGGWPWEN | 443 |
| 71p88P1D9 | GWVAGVGW | 434 | DGGGGWRWEN | 444 |
| 71p88P1B12 | GWIEGAGW | 435 | DGGWRSRWEN | 445 |
| 71p88P1B4 | GWLEGYGW | 265 | DGGAERAWEN | 446 |

Clones were sub-cloned into the pANA19 vector and expressed in bacteria for production and purification of ATRIMERS™ as detailed in Example 12. Cell killing was measured on ST486 (Burkitt's lymphoma), A2780 (ovarian carcinoma), Colo205 (colon carcinoma) and H2122 (non small cell lung carcinoma) cell lines. Briefly 1−5×10⁴ cells per well were incubated with the purified DR4 ATRIMERS™ (20 μg/ml) or TRAIL (0.2 μg/ml, R&D systems) as described in Example 14. Cell viability was measured using the Vialight Plus kit (Lonza), and data expressed as % killing is shown in Table 9.

TABLE 9
Killing of cancer cell lines by DR4 specific ATRIMERS ™ (%
of TRAIL activity)

| | ST486 % | A2780 % | Colo205 % | H2122 % |
|---|---|---|---|---|
| Clone | killing | killing | killing | killing |
| 71p88P1B3 | 88% | 6% | 42% | 0% |
| 71p88P1G4 | 75% | −8% | 24% | 5% |
| 71p88P1G1 | 78% | −9% | 12% | 0% |
| 71p88P1F2 | 58% | −8% | −18% | −5% |
| 71p88P1A1 | 57% | −5% | −1% | −2% |
| 71p88P1G11 | 45% | −1% | 1% | 4% |
| 71p88P1A3 | 41% | −5% | −7% | −3% |
| 71p88P1B9 | 34% | 5% | 7% | 3% |
| 71p88P1D9 | 28% | −12% | −21% | −3% |
| 71p88P1B12 | 26% | −4% | −10% | −1% |
| 71p88P1B4 | −15% | −7% | −25% | −5% |

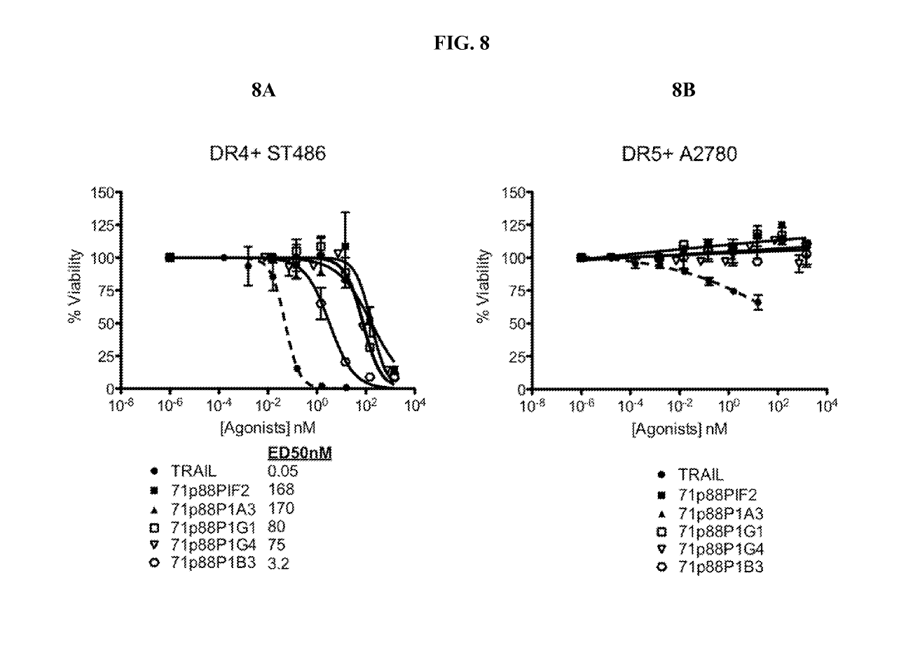

ED50 values were generated for the best clones as shown in FIG. 8 and Table 10.

TABLE 10
Loop1 and Loop4 sequences and ED50 values for
DR4 agonist clones

| | Loop 1 | Loop 4 | ED50 |
|---|---|---|---|
| Clone | (SEQ ID NO) | (SEQ ID NO) | ST486 |
| 29p61P1E11 | GWLEGVGW (261) | DGGWPFSNEN (315) | 217 nM |
| 71p88P1B3 | GWLEGSGW (428) | DGGVQWRWEN (436) | 3.2 nM |
| 71p88P1G4 | GYMTGVGW (429) | DGGRSWKWEN (437) | 75 nM |
| 71p88P1G1 | GWMEGVGW (430) | DGGPPWRWEN (438) | 80 nM |
| 71p88P1F2 | GWLEGSGW (428) | DGGFPARWEN (439) | 168 nM |
| 71p88P1A3 | GYLAGTGW (432) | DGGRVLAWEN (443) | 170 nM |

A strong consensus sequence (GWLEGv/sG) was observed in loop 1 of many of the binding clones with activity. However this sequence alone did not confer activity, but required additional sequences in loop 4.

### Example 15

### Construction of the Affinity Maturation Library of Clone 29p61P1E11

To obtain more potent DR4 specific ATRIMERS™ affinity maturation of the initial DR4 agonist clone 29p61P1E11 was intimated. Analysis of the Loop sequences strongly suggested that the unique Loop 4 conferred the agonist activity of this ATRIMER™. Therefore a library was built in which 6 amino acid positions in Loop 3 of 29p61P1E11 (ETEITA) were replaced with random amino acids encoded by the nucleotides NNK NNK NNK NNK NNK NNK (SEQ ID NO: 252). This was achieved by overlap PCR using the following primers:

1E11 L3AF (SEQ ID NO: 447):
GAGCGTGGGCAACGAGGCCGAGATCTGGCTGGGCCTCAACGGTTGGCTGG
AAGGCGTGGGT
1E11 L3AR (SEQ ID NO: 448):
CCAGTTCTTGTAGGCGATACGCGCGCCAGTCATATCCACCCAACCCACGC
CTTCCAGCCAACCGTTGAGG
1E11 L3BF (SEQ ID NO: 449):
ATCGCCTACAAGAACTGGNNKNNKNNKNNKNNKNNKCAACCCGATGGCGG
TTGGCCGTTCAGCAAC
1E11 L3BR (SEQ ID NO: 450):
CGCTTGTCGAACCACTTGCCGTTGGCGGCGCCAGACAGGACGGCGCAGTT
CTCGTTGCTGAACGGCCAACCG

The resulting fragments were gel purified, mixed and extended with the outer primers HuBglfor2 (SEQ ID NO: 257) and PstRev (SEQ ID NO: 203). The product of this reaction was gel purified and digested with BglII and PstI restriction endonucleases and cloned into similarly digested phage display vector pANA27. A library of 2.19×10⁹ was obtained; sequencing of several randomly selected clones showed diversity in the targeted region of Loop 3.

### Example 16

### Affinity Maturation Panning Using Decreasing Amounts of Biotinylated DR4 on Magnetic Resin

Recombinant Human TRAILR1 (DR4)/Fc chimera was biotinylated and purified using a Sulfo-NHS micro biotinylation kit (Thermo-Scientific). Phage were generated from affinity matured libraries and resuspended in a casein buffer containing 0.5% boiled casein, 0.025% Tween 20 in PBS with added EDTA-free protease inhibitors (Roche). Two 50 μl aliquots of streptavidin coated magnetic beads (Dynalbeads/Invitrogen) were washed and blocked in 0.5% boiled casein in PBS with 1% Tween 20. A 150 μl aliquot of the phage preparation was preincubated for 30 min at 37° C. with one aliquot of blocked streptavidin resin to remove non-specific and streptavidin binders. Pre-bound phage was then transferred to a new vessel in which it was incubated in the presence of 1 μg of biotinylated TRAILR1 (DR4)/Fc chimera for 120 min at 37° C. After binding of phage and biotinylated TRAILR1 (DR4)/Fc the material was added to the remaining aliquot of blocked Streptavidin resin and allowed to bind for 30 minutes at 37° C. Using a magnetic stand the beads were then washed 5 times with 0.5% boiled casein, 0.025% Tween 20 in PBS. Phage were eluted with glycine pH 2.0, neutralized with 2 M Tris pH 11.5 and used to infect SS320 E. coli cells (Lucigen), as described above. For all subsequent rounds of panning the number of washes was increased to 10. The amount of biotinylated TRAILR1 (DR4)/Fc target was decreased 10-100 fold for each successive round. Clones obtained from the affinity mature panning of the 29p61P1E11 library were screened for DR4 specific binding by Elisa and sequenced as described above. Sequences are shown in Table 11.

TABLE 11
Loop 3 sequences of 29p61P1E11 affinity
matured clones

| Clone | Loop 3 SEQ | SEQ ID NO |
|---|---|---|
| 119p83P1H1 | NWTQRHSGQ | 451 |
| 119p94P1B5 | NWARHINEQ | 452 |
| 119p83P1A7′ | NWYSWPKLQ | 453 |
| 119p83P1H4 | NWSKVRLEQ | 454 |
| 119p83P1A3 | NWVAKDHEQ | 455 |
| 119p83P1C12 | NWNSNVVLQ | 456 |
| 119p94P1G7 | NWGWSARVQ | 457 |
| 119p94P1D8 | NWGWMDSKQ | 458 |
| 119p94P1B2 | NWWFPTLSQ | 459 |
| 119p83P1D9L4 | NWEHPEPWQ | 460 |
| 119p83P1C6L4 | NWEPPEPLQ | 461 |
| 119p94P1B6 | NWHPQGDRQ | 462 |
| 119p94P1H10 | NWSTAQNGQ | 463 |
| 119p94P1D2 | NWLDVTKTQ | 464 |
| 119p94P1C1 | NWAISDERQ | 465 |
| 119p94P1B4 | NWAEVPFFQ | 466 |
| 119p94P1B8 | NWWSYWDTQ | 467 |
| 119p94P1F4 | NWAAVTMEQ | 468 |
| 119p94P1E10 | NWRVPSLRQ | 469 |
| 119p94P1H3 | NWSLSWHPQ | 470 |
| 119p94P1E7 | NWIWSRIEQ | 471 |
| 119p94P1D4 | NWAAFPVEQ | 472 |
| 119p94P1D11 | NWGSTGEKQ | 473 |
| 119p94P1G1 | NWGEVIAPQ | 474 |
| 119p94P1A4 | NWFAEFFLQ | 475 |
| 119p83P1D10 | NWGRRRNLQ | 476 |
| 119p83P1G12 | NWGSYGPFQ | 477 |
| 119p83P1H12 | NWGTHISSQ | 478 |
| 119p83P1H5 | NWGTGVMGQ | 479 |
| 119p83P1H8 | NWGGSISAQ | 480 |
| 119p83P1C9 | NWGGEVLLQ | 481 |
| 119p83P1H3 | NWSEDRPGQ | 482 |
| 119p83P1A9 | NWVYRPGMQ | 483 |
| 119p83P1H7 | NWVNHGVGQ | 484 |
| 119p83P1A12 | NWQPGLWRQ | 485 |
| 119p83P1D11 | NWQVHARSQ | 486 |
| 119p83P1C11 | NWAMHYYWQ | 487 |
| 119p83P1A11 | NWDAPVSGQ | 488 |
| 119p83P1F12 | NWFIPADRQ | 489 |
| 119p83P1G3 | NWYVRSEGQ | 490 |
| 119p83P1D9Q | NWEHPEPWHQ | 491 |

Clones were then sub-cloned into pANA19 for expression and purification as described above. Cell killing activity was measured as described above, and binding affinity was measured by Biacore. Results are shown in Table 12 below.

TABLE 12
ED50 values and binding affinities of
29p61P1E11 affinity matured clones

| | ED50 (nM) | On Rate | Off Rate | |
|---|---|---|---|---|
| Clone | ST486 | (1/Ms) | (1/s) | K_{D} (nM) |
| 29p61P1E11 | 217 ± 71 | 9.38E+4 | 3.02E−4 | 3.2 |
| 119p83P1H4 | 0.13 | 1.38E+5 | 2.21E−5 | 0.16 |
| 119p94P1B5 | 6.6 ± 4.8 | 3.6E+5 | 4.06E−4 | 1.11 |
| 119p83P1H1 | 13 ± 6.1 | 2.21E+5 | 3.29E−4 | 1.42 |
| 119p94P1G7 | 25.5 ± 9.2 | 3.49E+5 | 7E−4 | 2.41 |
| 119p94P1D8 | 30.5 ± 19.1 | 4E+5 | 5.42E−4 | 1.31 |
| 119p94P1B4 | 127 | 1.85E+5 | 5.2E−4 | 2.82 |
| 119p94P1C1 | 251 | 1.41E+5 | 5.71E−4 | 1.85 |
| 119p94P1F4 | 308 | 1.25E+5 | 8.92E−4 | 2.96 |
| 119p83P1D9 | 766.5 ± 222.7 | 3.47E+5 | 6.59E−4 | 1.9 |

The ATRIMER™ 71p881B3 showed higher agonist activity, as compared to the ATRIMER™ 29p61P1E11 (prior to affinity maturation of the loop 3 of 29p61P1E11). The ATRIMER™ 71p881B3 has modified loops 1 and 4 which confer specific binding and activity through DR4.

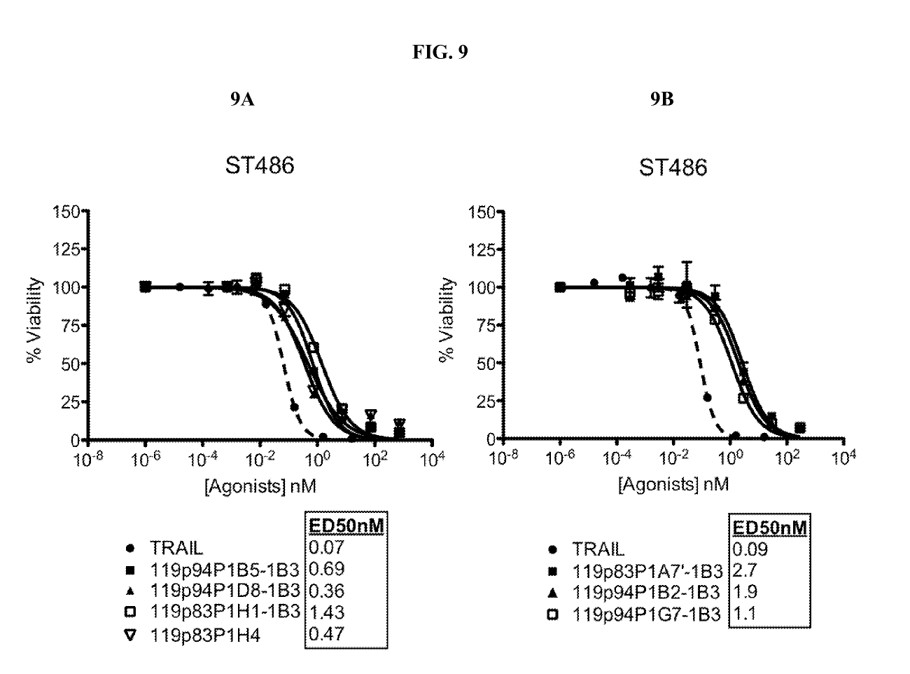

In order to increase the potency of ATRIMER™ 71p881B3, the loop 3 sequences obtained from the affinity maturation of ATRIMER™ 29p61P1 μl were taken from the affinity matured clones: 119p94P1B5 (SEQ ID NO: 452), 119p94P1D8 (SEQ ID NO:458), 119p83P1H1 (SEQ ID NO:451), 119p94P1G7 (SEQ ID NO:457), 119p94P1B2 (SEQ ID NO:459), and 119p83P1A7′ (SEQ ID NO:453) (see Table 11), and sub-cloned into the loop 3 position of the clone 71p881B3. These new hybrid clones were expressed in bacteria and tested for agonist activity in cell based assays as described above on the DR4 expressing cell line ST486 and showed agonist activity. The results are presented in FIG. 9. The ATRIMERS™ are labeled to represent the loop 1 and loop 4 modifications of ATRIMER™ 71p881B3 and the loop 3 sequences from the 29p61P1E11 affinity matured clones.

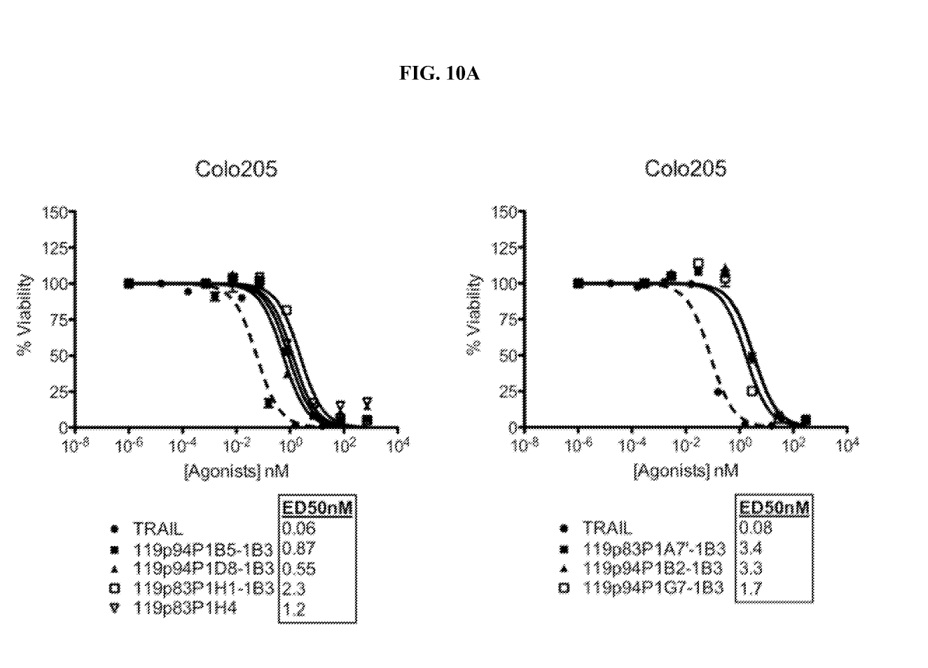

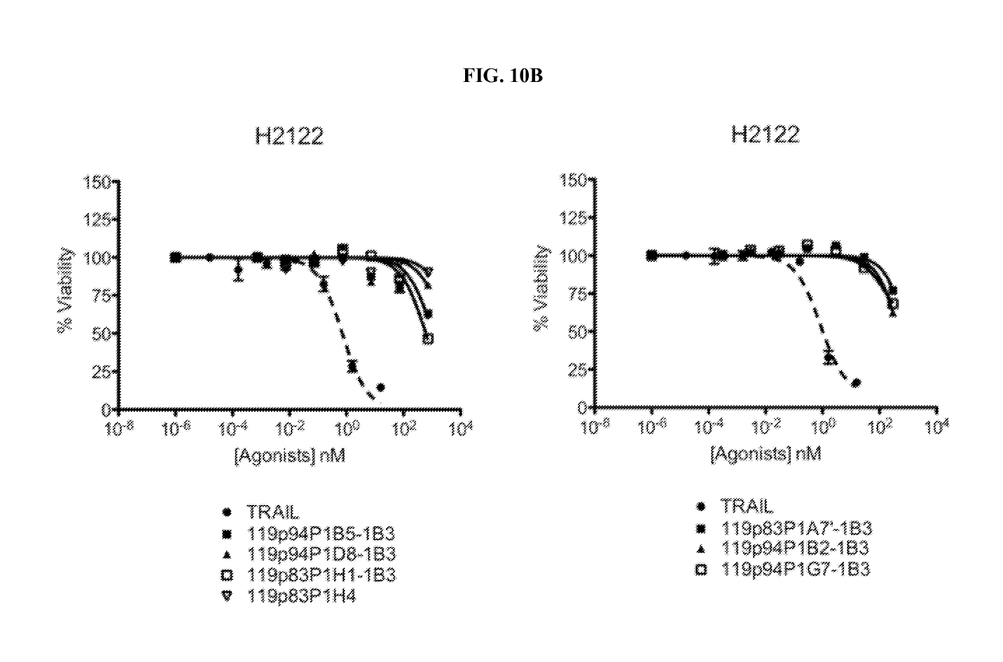

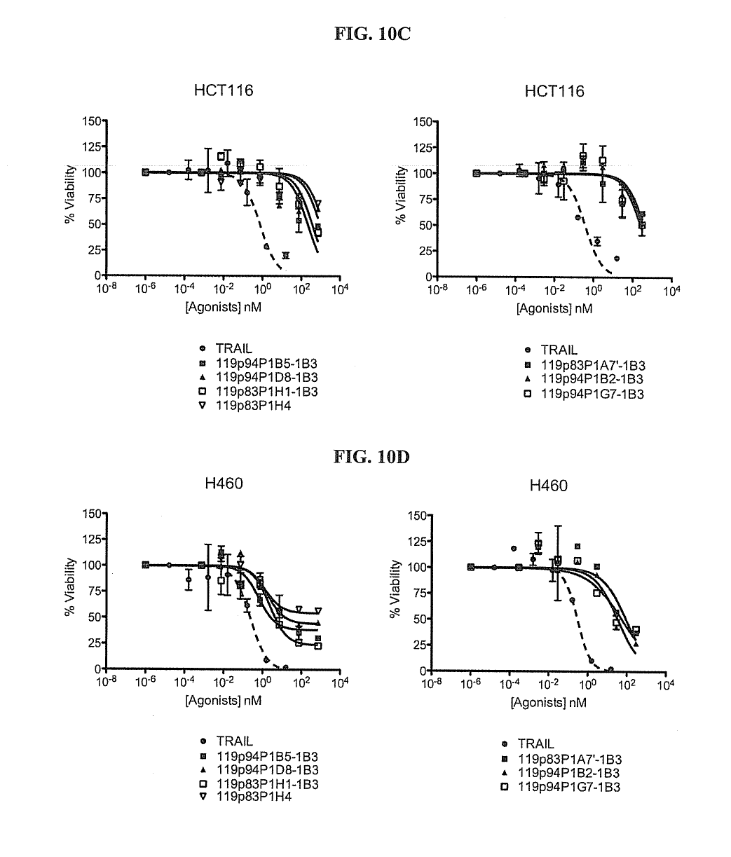

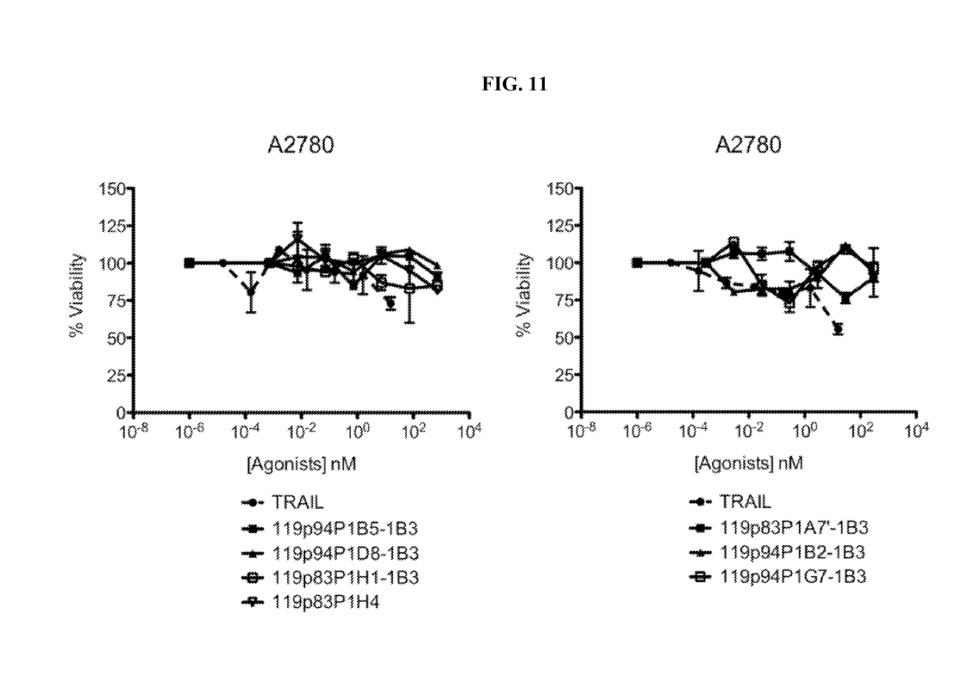

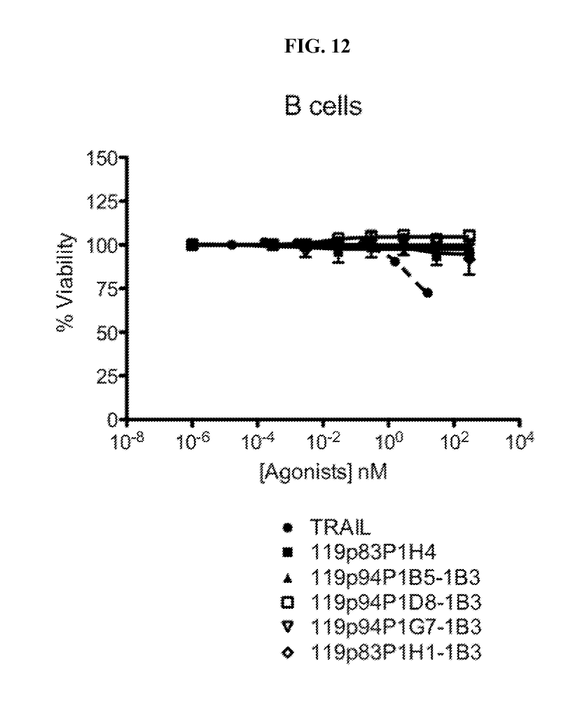

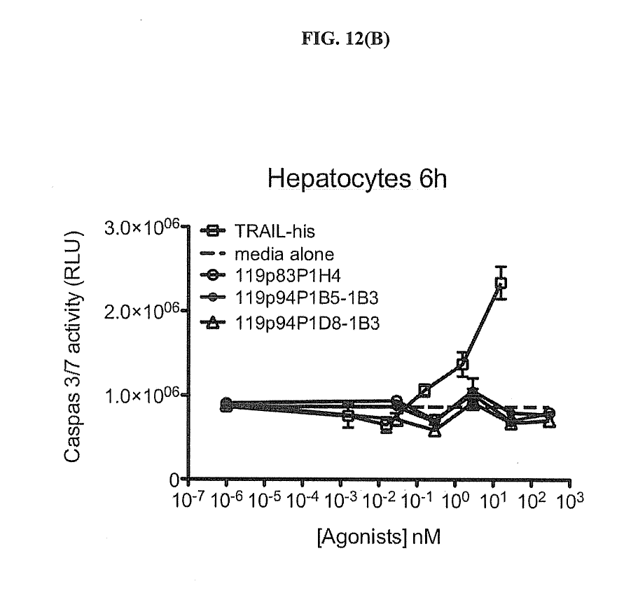

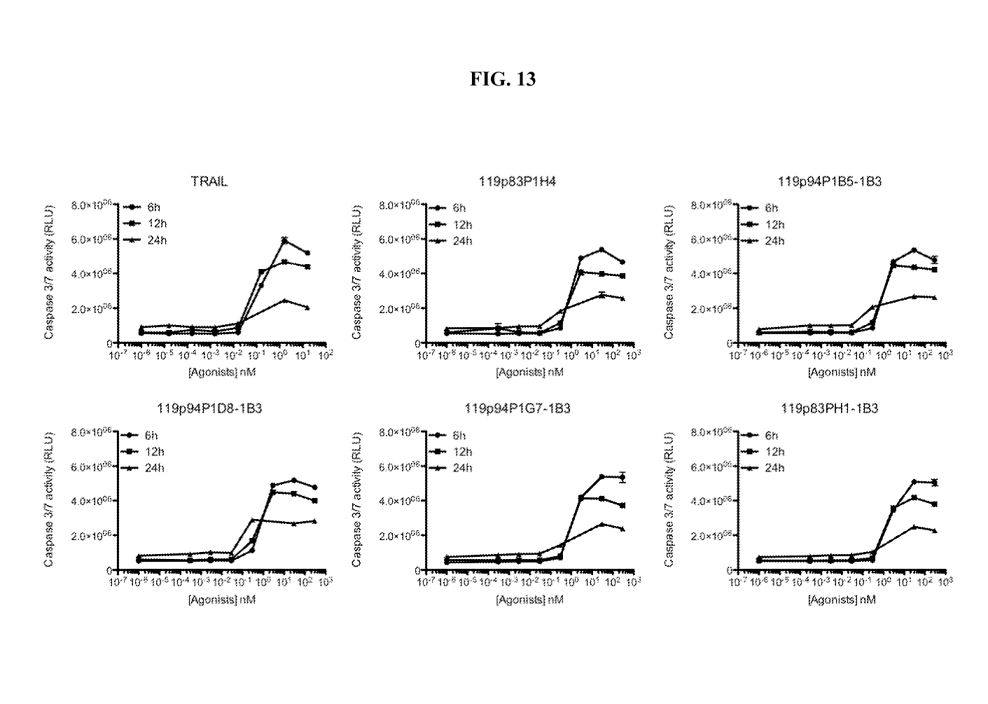

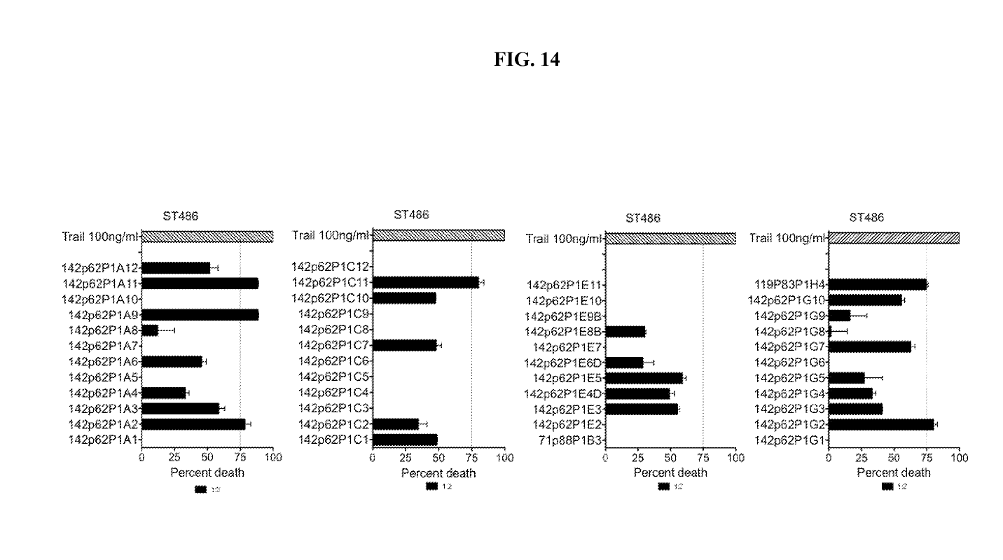

In addition to the cell line ST486, cell killing was observed on Colo-205, HCT-116, H2122 and H460 cancer cell lines expressing the DR4 receptor in similar assays (FIGS. 10A, 10B, 10C and 10D). ATRIMERS™ specifically killed DR4-expressing cells, as agonist activity was not observed on the DR4 negative cell line A2780 (FIG. 11). The ATRIMERS™ were also tested for agonist activity on normal B cells (FIG. 12A) and primary hepatocytes (FIG. 12B). Despite DR4 expression by normal B cells, these cells were not killed by DR4 ATRIMER™ agonists, indicating their selectivity on cancer cells versus normal cells (FIGS. 12A and 12B). Cell killing was demonstrated to be through the Caspase pathway in these assays measuring Caspase activity using the Caspase-Glo3/7 assay kit (Promega) (see Example 27). This is consistent with TRAIL stimulation of cell killing through Caspase activation (See FIG. 13).

### Example 17

### Construction of the clone71p881B3 Affinity Maturation Library

To select for affinity matured clones within the confines of the specific sequence of clone 71p881B3, improvement the binding affinity and agonist activity of this clone were also sought through a very similar approach to that used above for clone 29p61P1E11 (1E11). A library was constructed in which 6 amino acid positions in Loop 3 of 71p881B3 (ETEITA; SEQ ID NO: 255) were replaced with random amino acids encoded by the nucleotides NNK NNK NNK NNK NNK NNK (SEQ ID NO: 252). Overlap PCR was performed as above but with the following oligonucleotides:

1B3L3AF (SEQ ID NO: 492):
GAGCGTGGGCAACGAGGCCGAGATCTGGCTGGGCCTCAACGGTTGGCTGG
AAGGCTCTGGT
1B3L3AR (SEQ ID NO: 493):
CCAGTTCTTGTAGGCGATACGCGCGCCAGTCATATCCACCCAACCAGAGC
CTTCCAGCCAACCGTTGAGG
1B3L3BF (SEQ ID NO: 494):
ATCGCCTACAAGAACTGGNNKNNKNNKNNKNNKNNKCAACCCGATGGCGG
TGTTCAGTGGAGGTGG
1B3L3BR (SEQ ID NO: 495):
CGCTTGTCGAACCACTTGCCGTTGGCGGCGCCAGACAGGACGGCGCAGTT
CTCCCACCTCCACTGAACACCG

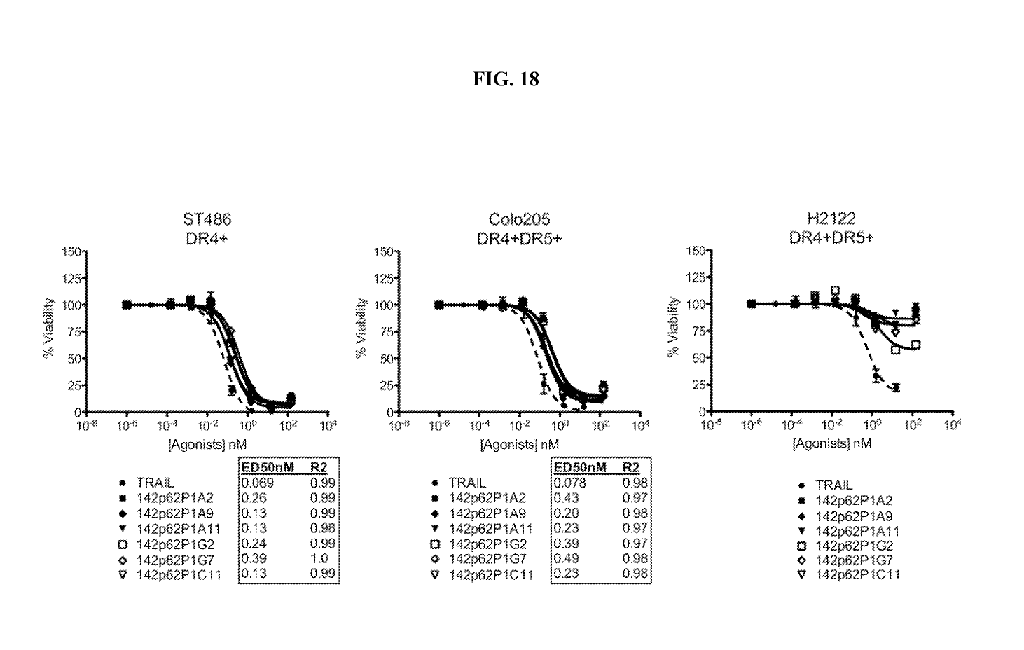

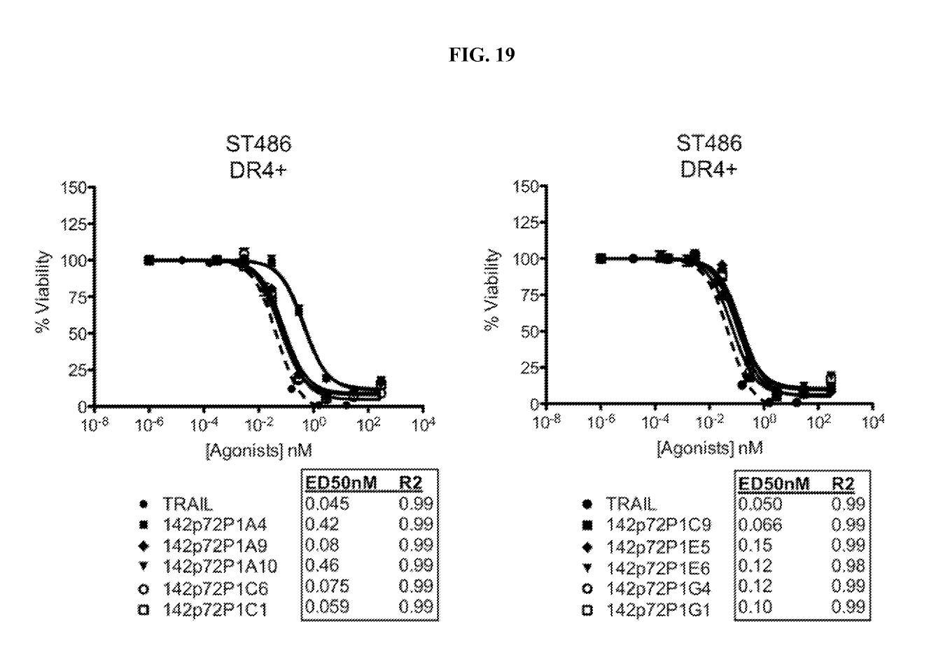

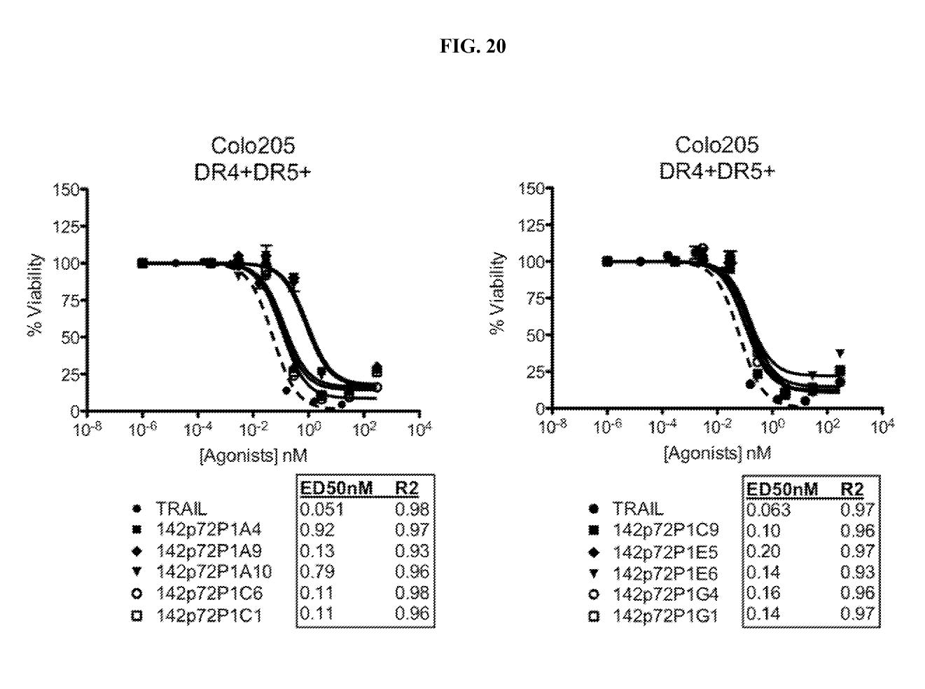

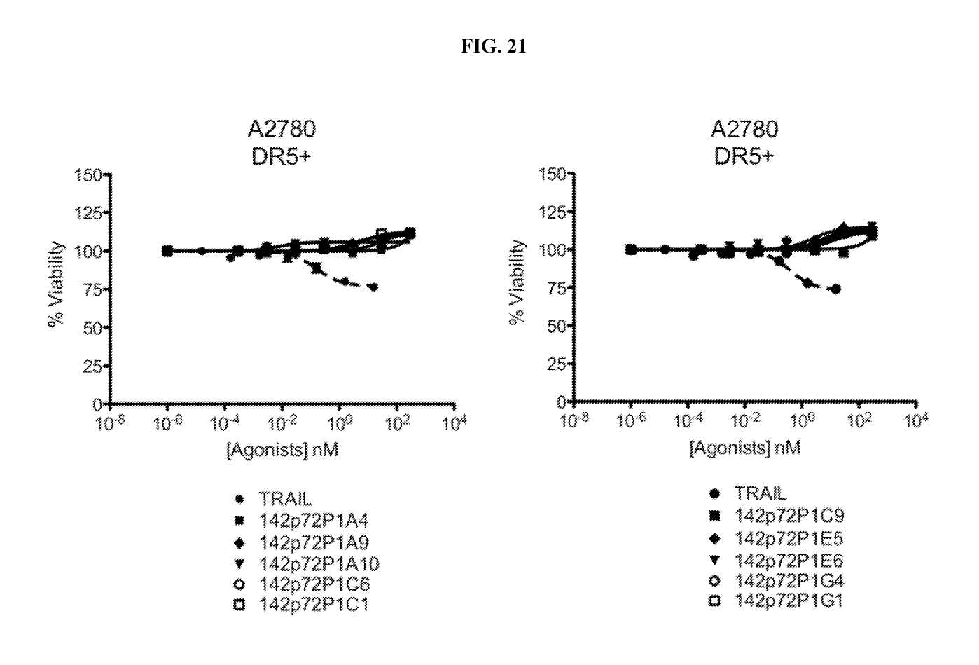

The resulting fragment was cloned into pANA27 as above. A library of 2.65×10⁹ was obtained and sequencing of randomly selected clones showed diversity in the targeted region of Loop 3. The library was panned as detailed above for the affinity matured library of clone 29p61P1E11. The panning round 4 and round 5 pools of clones were sub-cloned into the mammalian expression vector pANA20. Individual clones were then picked, miniprep DNA isolated and transiently transfected into 293 cells for production of the ATRIMERS™. Raw unpurified supernatants containing the ATRIMERS™ were tested for activity in cell based assays. Clones which showed agonist activity were sequenced, and the loop 3 amino acid sequences obtained as shown in Table 13 below. Clones which showed strong activity were then sub-cloned into pANA19. The proteins from these subclones were then produced in, and purified from bacteria. Cell based killing activity was then measured (FIGS. 18-21) in comparison to TRAIL.

TABLE 13

| | Cell killing | | |
|---|---|---|---|
| | activity | | |
| | (% activity | | |
| Clone | of TRAIL) | Loop3 SEQ | SEQ ID NO |
| 142p62P1A2 | 75% | NWGDQRLAQ | 496 |
| 142p62P1A3 | 60% | NWADERRNQ | 497 |
| 142p62P1A9 | 90% | NWADKRWLQ | 498 |
| 142p62P1A11 | 90% | NWKDDRFNQ | 499 |
| 142p62P1A12 | 50% | NWLDPRMGQ | 500 |
| 142p62P1C1 | 50% | NWYSDYLNQ | 501 |
| 142p62P1C10 | 50% | NWHYQKYIQ | 502 |
| 142p62P1C11 | 80% | NWALDRYNQ | 503 |
| 142p62P1E3 | 50% | NWGRPELAQ | 504 |
| 142p62P1E5 | 60% | NWANPSFMQ | 505 |
| 142p62P1G2 | 80% | NWADERFLQ | 506 |
| 142p62P1G7 | 65% | NWGRRELAQ | 507 |
| 142p72P1A10 | 70% | NWYDPVYDQ | 539 |
| 142p72P1A4 | 70% | NWASEVFQQ | 540 |
| 142p72P1A9 | 90% | NWADARWDQ | 541 |
| 142p72P1C1 | 95% | NWADDRWNQ | 542 |
| 142p72P1C5 | 70% | NWAYSKWNQ | 543 |
| 142p72P1C6 | 85% | NWANQRWNQ | 544 |
| 142p72P1C9 | 95% | NWGDPRWSQ | 545 |
| 142p72P1E5 | 70% | NWANLRFNQ | 546 |
| 142p72P1E6 | 75% | NWADPTWSQ | 547 |
| 142p72P1G1 | 95% | NWGDSRFMQ | 548 |
| 142p72P1G2 | 95% | NWGNPRWGQ | 549 |
| 142p72P1G4 | 95% | NWGTPRLAQ | 550 |
| 142p74P1A1 | 80% | NWAPGVVAQ | 551 |
| 142p74P1A7 | 60% | NWGHGDLWQ | 552 |
| 142p74P1E1 | 75% | NWYNASFFQ | 553 |
| 142p74P1E4 | 80% | NWGDARFGQ | 554 |
| 142p74P1G4 | 60% | NWAEARLWQ | 555 |
| 142p74P1G5 | 90% | NWAEARWWQ | 556 |
| 142p74P1G6 | 90% | NWAVDTFNQ | 557 |
| 142p74P1C1 | 95% | NWARDIFNQ | 558 |
| 142p74P1C2 | 80% | NWGGWLADQ | 559 |
| 142p74P1C3 | 90% | NWGDARWAQ | 560 |
| 142p74P1C5 | 80% | NWADERWSQ | 561 |
| 142p74P1C7 | 80% | NWADPKYNQ | 562 |

A number of clones were produced with desired sequences in loops 1, 3 and 4 of an the polypeptide sequences of ATRIMERs™ based upon the human tetranectin scaffold. Cell killing was measured as described above.

TABLE 14

| | Loop 1 | Loop 3 | Loop 4 | | |
|---|---|---|---|---|---|
| | sequence | sequence | sequence | EC₅₀ | EC₅₀ |
| Clone Name | (SEQ ID NO) | (SEQ ID NO) | (SEQ ID NO) | ST486 | Colo205 |
| 142p62P1A2 | GWLEGSGW | NWGDQRLAQ | DGGVQWRWEN | 0.30 nM | 0.39 nM |
| | (428) | (496) | (436) | | |
| 142p62P1G2 | GWLEGSGW | NWADERFLQ | DGGVQWRWEN | 0.20 nM | 0.32 nM |
| | (428) | (506) | (436) | | |
| 142p72P1C9 | GWLEGSGW | NWGDPRWSQ | DGGVQWRWEN | 0.03 nM | 0.18 nM |
| | (428) | (545) | (436) | | |
| 65p114P1A3 | GWLEGSGW | NWADPKWSQ | DGGVQWRWEN | 0.05 nM | 0.11 nM |
| | (428) | (569) | (436) | | |
| 65p114P1B2 | GWLEGSGW | NWFHDRFNQ | DGGVQWRWEN | 0.09 nM | 0.18 nM |
| | (428) | (570) | (436) | | |
| rhTRAIL-His | NA | NA | NA | 0.06 nM | 0.06 nM |

### Example 18

### Panning of NEB Peptide Libraries on Human DR5 and Identification of a DR5 Specific Peptide

Panning of peptide libraries was performed using the New England Biolabs (NEB) Ph.D. Phage Display Libraries. Panning was performed on DR5/Fc antigen-coated (R&D Systems) wells prepared fresh the night before bound with 3 μg of the carrier free target antigen diluted in 150 μL of 0.1M NaHCO₃ pH 8.6 per well. Duplicate wells were used in each round. Antigen plates were incubated overnight at 4° C. then for 1 hour at 37° C. The antigen was removed and the well was then blocked with 0.5% boiled Casein in PBS pH 7.4 for 1 hr at 37° C. prior to panning The Casein was then removed and wells were then washed 6× with 300 μL of TBST (0.1% Tween), then phage were added. Since target antigens were expressed as Fc fusion proteins, prior to target antigen binding, phage supernatants were pre-bound for 1 hr to antigen wells with human IgG1 Fc to remove Fc binders (during rounds 2 through 4). Fc antigen bound wells were prepared similar to DR5/Fc antigen bound wells as detailed above.

For the initial round of panning, 100 μL of TBST(0.1% Tween) was added to each well and 5 ul of each of the 3 NEB peptide libraries (Ph.D.-7, Ph.D.-12, and Ph.D.-C7C) were added to each well. The plate was rocked gently for 1 hr at room temperature, then washed 10× with TBST(0.1% Tween). Bound phage were eluted with 100 μL of PBS containing soluble DR5/Fc target antigen at a concentration of 100 μg/ml. Phage were eluted for 1 hr rocking at room temperature. Eluted phage were then removed from the wells and used to infect 20 mls of ER2738 bacteria at an OD₆₀₀ₙₘ of 0.05 to 0.1, and grown shaking at 250 rpm at 37° C. for 4.5 hrs. Bacteria were then spun out of the culture at 12K×G for 20 min at 4° C. Bacteria were transferred to a fresh tube and re-spun. The supernatant was again transferred to a fresh tube and the Phage were precipitated by adding ⅙^{th} the volume of 20% PEG/2.5M NaCl. Phage were precipitated overnight at 4° C. The following day the precipitated phage were spun down at 12K×G for 20 min at 4° C. The supernatant was discarded and the phage pellet re-suspended in 1 ml of TBST(0.1% Tween). Residual bacteria were cleared by spinning in a microfuge at 13.2K for 10 minutes at 4° C. The phage supernatant was then transferred to a new tube and re-precipitated by adding 1/6^{th} the volume of 20% PEG/2.5M NaCl, and incubating at 4° C. on ice for 1 hr. The precipitated phage were spun down in a microfuge at 13.2K for 10 minutes at 4° C. The supernatant was discarded and the phage pellet re-suspended in 200 μL of TBS. Subsequent rounds of panning were performed similar to round 1 with the exception phage were pre-bound for 1 hr to Fc coated wells and that 4 μL of the amplified phage stock from the previous round were used per well during the binding. In addition the tween concentration was increased to 0.5% in the TBST used during the 10 washes.

Phage ELISA

Panning was performed using the ER2738 strain of bacteria for at least four rounds. At each round of panning sample titers were taken and plated using top agar on LB/×gal plates to obtain plaques. To screen for specific binding of phage clones to the receptor target, individual plaques were picked from these titer plates from the later rounds of panning and used to infect ER2738 bacteria at an OD₆₀₀ₙₘ of 0.05 to 0.1, and grown shaking at 250 rpm at 37° C. for 4.5 hrs. Then stored at 4° C. overnight.

On day 2, cultures were spun down at 12K×G for 20 min at 4° C., and supernatants containing the phage were blocked with 3% milk/PBS for 1 hr at room temperature. An initial Phage ELISA was performed using 75-100 ng of DR5/Fc antigen bound per well. Non-specific binding was measured using wells containing 75-100 ng of human IgG1 Fc petr well. DR5/Fc antigen (R&D Systems)-coated wells and IgG1 Fc coated wells were prepared fresh the night before by binding the above amount of antigen diluted in 100 μL of PBS per well. Antigen plates were incubated overnight at 4° C. then for 1 hour at 37° C., washed twice with PBS/0.05% Tween 20 and twice with PBS, and then blocked with 3% milk/PBS for 1 hr at 37° C. prior to the ELISA. Blocked phage were bound to blocked antigen-bound plates for 1 hr then washed twice with 0.05% Tween 20/PBS and then twice more with PBS. A HRP-conjugated anti-M13 secondary antibody diluted in 3% milk/PBS was then applied, with binding for 1 hr and washing as described above. The ELISA signal was developed using 90 μL TMB substrate mix and then stopped with 90 μL 0.2 M sulfuric acid, then ELISA plates were read at 450 nM. Secondary ELISA screens were performed on the positive binding clones identified, screening against additional TRAIL receptors and decoy receptors to test for specificity (DR4, DR5, DcR1 and DcR2). Secondary ELISA screens were performed similarly to the protocol detailed above.

DR5 specific binding clone

An example of the amino acid sequence of a peptide from the NEB Ph.D.—C7C phage library selected for specific binding to the DR receptor is detailed below in Table 15.

TABLE 15

| | | Peptide |
|---|---|---|
| | Peptide | SEQ |
| Clone | Sequence | ID NO |
| 088-13.1H3 | ACFPIMTLHCGGG | 410 |

### Example 19

### Cloning of a Trimeric Displayed Peptide Library

In order to select for peptides which would bind in a trimeric conformation when fused to the trimerization domain of human tetranectin, a new peptide phage display library was constructed. In this library the C-terminus of the trimerization domain was fused to the N terminus of gene III of the phage with an amber stop codon at the junction. This allows for both the trimerization domain/gene III fusion protein as well as the trimerization domain alone to be produced, so that a trimeric protein fused through a single gene III coat protein could be assembled and displayed on the surface of the phage particle. In addition the N terminus of the trimerization domain is fused with a peptide consisting of 15 random amino acids, thus allowing the random peptide library to be displayed at a trimer (FIG. 15).

The phage vector pCANTAB 5E was first modified in order to replace the CTLD domain by the trimerization domain of tetranectin and to introduce restriction sites for the cloning of degenerate oligos.

Introduction of KpnI and NheI sites in pCANTAB

Introduction of KpnI and NheI sites was performed by PCR using primers:

(SEQ ID NO: 508)

CAN-KPN, 5′TTCGCAATTCCTTTAGTGGTACCTTTCTATTCTCACTCT
GCTAGCATGGCCGCCCTCCAG-3′
and

(SEQ ID NO: 509).

CAN-CTLD-R, 5′AGTCTATGCGGCACGCGGTT-3′

Insertion of the Trimerization Domain into pCANTAB
The trimerization domain of tetranectin was first amplified by PCR using primers: TD-NHE, 5′-GGTGGAGCTAGCGTTGTGAACACAAAGATGTTTGAG-3′ (SEQ ID NO: 510) and TD-NOT, 5′-GTGCACTGCGGCCGCCTTCAGGCAGACCGTCTGGAGGGC-3′ (SEQ ID NO: 511) and pANA14 as template.

Insertion of Degenerate Oligonucleotides into pCANTAB-TD
The DNA fragment containing a completely randomized 15 mer region was amplified by PCR using primers: DGP-F, 5′-CTTTCTATTCTCACTCC (NNK)¹⁵GGTGGCGGTTCGGCTGAAG-3′ (SEQ ID NO: 512) and CAN-CTLD-R, 5′-AGTCTATGCGGCACGCGGTT-3′ (SEQ ID NO: 513). DGP-F is a degenerate oligo that begins with a 17 base pairs sequence complimentary to the region preceding the insertion point into the vector pCANTAB and contains 15 random codons. The codons were designed to be NNK where N is all four nucleotides and K is G or T. This DNA template was further amplified by a second PCR using a forward primer that will introduce a KpnI site: TD-KPN, 5′-AACCTGGTACCTTTCTATTCTCACTCC-3′ (SEQ ID NO: 514). Both the DNA fragment and pCANTAB vector were digested with KpnI and NheI and ligated.

The estimated titer of this peptide library was 4×10⁷. Forty-four random clones were sequenced to evaluate the quality of the library. About 50% of the clones showed a perfect sequence (sequences are in-frame and no mutations). Some clones contained a triple deletion. However these clones will be in-frame since this creates a deletion of one full codon. The other 50% of the clones contained 1, 2 or 4 base deletions in the random sequence. This was likely due to the synthesis of the oligos which cannot be PAGE purified because of the random sequence. However, the quality of this library is satisfactory with at least 2×10⁷ clones of expected sequence.

### Example 20

### Panning Peptide Trimerization Domain Library on DR5-Fc

The peptide trimer library was panned on human DR5-Fc for 4 rounds. One μg of DR5-Fc was used per well. Infections were performed using ER2738 cells. During the first round, plates were washed only once with buffer D. Plates were washed 5 times during the 2^{nd} panning round and 10 times on panning rounds 3 and 4. Fc competitor was added only starting at round 2. Elution was performed using target elution (3 μg of DR5-Fc per well).

Summary DR5 Binders Obtained from the Peptide Trimer Library

The peptide trimer library was panned on DR5. The sequences of all the clones are shown below. A total of 9 clones were obtained with 8 cyclic clones containing 2 cysteines residues and one linear clone without any cysteine (132p103P9E8). Most of the clones containing 2 Cys residues are separated by 3 aa. In the others clones the cysteines are separated by 5 or 6 aa.

132p105P10B1

(SEQ ID NO: 515)

FYPSVCLTSCASIQR
132p18P3A10

(SEQ ID NO: 516)

MHMTPPYLCRWGCAT
132p19P5D1

(SEQ ID NO: 517)

VVMNGPFLCRTPCLV
132p105P9A6

(SEQ ID NO: 518)

QGPTIMGPYLCTYGC
132p45P7G2

(SEQ ID NO: 519)

GGCLPYLTCRMGSVT
132p103P11E7-4

(SEQ ID NO: 520)

QMNCRPILTCKHRTL
132p103P11E7-1

(SEQ ID NO: 521)

QEGWTFSCMPYLTCR
132p104P9C10

(SEQ ID NO: 522)

WTASSKFCSRPFLTC
132p103P9E8

(SEQ ID NO: 523)

TKIDDNALVITQKARWR

The Pro and Leu residues appear to be very conserved among all the cyclic peptides. An aromatic residue (Tyr or Phe) is preferentially found in between Pro and Leu.

The sequence of the linear peptide is actually 17 aa and not 15 aa. This is due to the fact that this clone had one base deletion in the wobble sequence as well as 2 base deletions in the linker sequence. This created a sequence in which 2 extra aa are added (WR) to the 15 aa random sequence and the deletion of 2 aa (SG) in the linker sequence. Furthermore, this clone had a stop codon (indicated as a q) in the middle of its sequence.

All the nine DR5 peptide binders were subcloned into pANA14 (TN V17 for expression in mammalian cells), pANA13 (TN V17 for expression in E. coli) and pANA40 (FL TN for expression in E. coli). The DR5 peptides 132p18P3A10 (cyclic peptide) and 9E8 (linear peptide) fused at the N terminal of TN were combined with the CTLD loops of the best DR4 agonists (119p83P1H1, 119p94P1B5 and 119p83P1A7′) to produce bispecific ATRIMERS™.

Characterization of 132p18P3A10 Deletion Mutants and Alanine Scanning Mutants

1) Sequence of 132p18P3A10 Deletion Mutants and Alanine Scanning Mutants

In order to better characterize the 132p18P3A10 peptide, deletion mutants from the N terminus of the peptide as well as Ala substitution were carried out. All these constructs were subcloned into pANA40 vector for bacterial expression. These various mutations helped determine which amino acids are important as well as the length requirements for agonist activity of 132p18P3A10. The expression levels will also be monitored to check if any of these mutations will help with production levels. A summary of all constructs is shown below:

Deletion Mutants
132p18P3A10

(SEQ ID NO: 524)

MHMTPPYLCRWGCAT
132p18P3A10-D1

(SEQ ID NO: 525)

-HMTPPYLCRWGCAT
132p18P3A10-D2

(SEQ ID NO: 526)

--MTPPYLCRWGCAT
132p18P3A10-D3

(SEQ ID NO: 527)

---TPPYLCRWGCAT
132p18P3A10-D4

(SEQ ID NO: 528)

----PPYLCRWGCAT
132p18P3A10-D5

(SEQ ID NO: 529)

-----PYLCRWGCAT
132p18P3A10-D6

(SEQ ID NO: 530)

------YLCRWGCAT
Alanine Mutants
132p18P3A10-P5A

(SEQ ID NO: 531)

MHMTAPYLCRWGCAT
132p18P3A10-P6A

(SEQ ID NO: 532)

MHMTPAYLCRWGCAT
132p18P3A10-Y7A

(SEQ ID NO: 533)

MHMTPPALCRWGCAT
132p18P3A10-L8A

(SEQ ID NO: 534)

MHMTPPYACRWGCAT
132p18P3A10-R10A

(SEQ ID NO: 535)

MHMTPPYLCAWGCAT
132p18P3A10-W11A

(SEQ ID NO: 536)

MHMTPPYLCRAGCAT
132p18P3A10-G12A

(SEQ ID NO: 537)

MHMTPPYLCRWACAT
132p18P3A10-T15A

(SEQ ID NO: 538)

MHMTPPYLCRWGCAA

2) Characterization of 132p18P3A10 Deletion Mutants

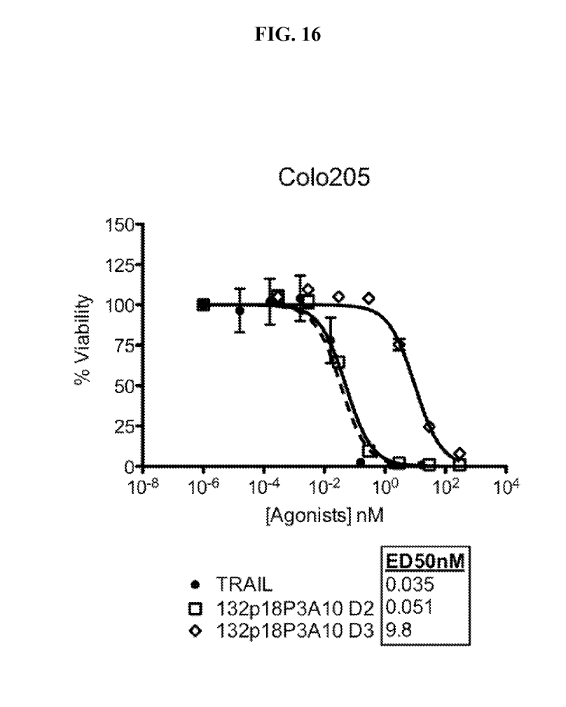

Only 2 deletion mutants were produced at sufficient levels in order to be tested in cell-based assays: 132p18P3A10-D2 and 132p18P3A10-D3. In this first set of experiments performed on Colo205 cells, 132p18P3A10-D2 completely retained its agonistic activity whereas 132p18P3A10-D3 agonistic activity was dramatically reduced by about 2 logs (FIG. 16).

3) Characterization of 3A10 Ala Mutants

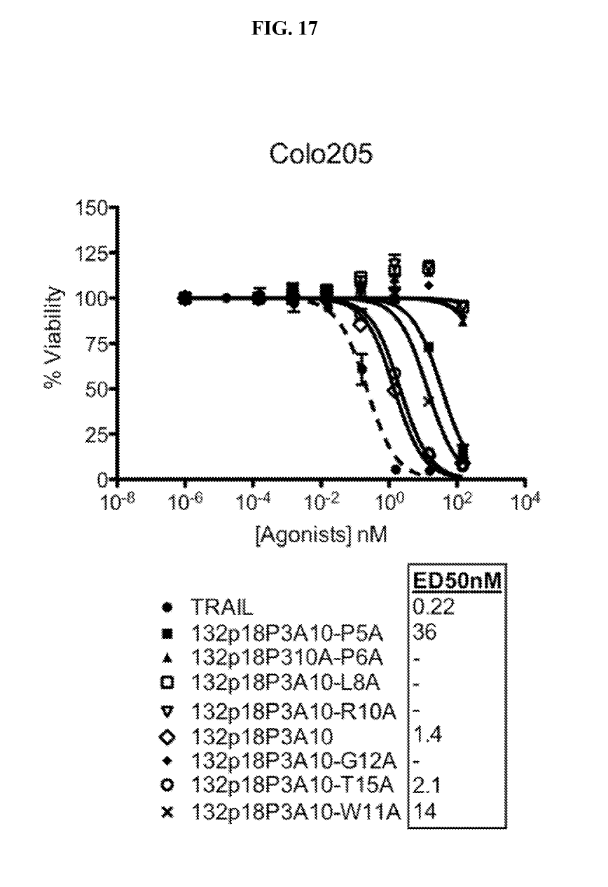

All of the Ala scan mutants were produced at sufficient levels and all could be tested in cell-based assays. In a first set of experiments performed on Colo205 and H2122 cells, it appears that all mutants were less active than 3A10 wt. The results showed that residues P6, L8, R10 and G12 are critical for 3A10 activity. Mutation of any of these residues almost completely abolishes 3A10 agonistic activity. This is likely because P6 and L8 were very conserved among all the DR5 peptides that were isolated. R10 and G12 mutation also abrogated 3A10 activity. However, these 2 residues are not as conserved as P6 and L8. For instance, in the clone 9A6 which is as good as 3A10, if not better, a Thr residue is found at position 10 instead of Trp. Mutation of P5, T15 and W11 also reduced 3A10 activity although not as dramatically as the other residues (FIG. 17). Therefore, a polypeptide containing the sequence XXXXXPXLXRXGXXX (SEQ ID NO: 563), wherein X is any amino acid, could serve as an efficient agonist to DR5.

### Example 21

### Plasmid Construction of Trimeric TRAIL Receptor Agonists and Trimeric CTLD-Derived TRAIL Receptor Agonists

The various versions of trimeric TRAIL receptor agonists and trimeric CTLD-derived TRAIL receptor agonists from phage display or from peptide-grafted, peptide-trimerization domain (TD) fusions, peptide-TD-CTLD fusion, or their various combinations are sub-cloned into bacterial expression vectors (pT7 in house vector, or pET, NovaGen) and mammalian expression vectors (pCEP4, pcDNA3, Invitrogen) for small scale or large-scale production.

Primers are designed to PCR amplify DNA fragments of binders/agonists from various functional display vectors from Example 1. Primers for the 5′-end are flanked with BamH I restriction sites and are in frame with the leader sequence in the vector pT7CIIH6. 5′ primers also can be incorporated with a cleavage site for protease Granzyme B or Factor Xa. 3′primers are flanked with EcoRI restriction sites. PCR products are digested with BamHI/EcoRI, and then ligated into pT7CIIH6 digested with the same enzymes, to create bacterial expression vectors pT7CIIH6-TRAILa.

The TRAIL receptor agonist DNAs can be sub-cloned into vector pT7CIIH6 or pET28a (NovoGen), without any leader sequences and 6×His. 5′ primers are flanked with NdeI restriction sites and 3′ primers are flanked with EcoRI restriction sites. PCR products are digested with NdeI/EcoRI, and ligated into the vectors digested with the same enzymes, to create expression vectors pT7-TRAILa and pET-TRAILa.

The TRAIL receptor agonist DNAs can be sub-cloned into vector pT7CIIH6 or pET28a (NovoGen), with a secretion signal peptide. Expressed proteins are exported into bacterial periplasm, and secretion signal peptide is removed during translocation. 5′ primers are flanked with NdeI restriction sites and the primers are incorporated into a bacterial secretion signal peptide, PelB, OmpA or OmpT. 3′ primers are flanked with EcoRIrestriction sites. A 6×His tag coding sequence can optionally be incorporated into the 3′ primers. PCR products are digested with NdeI/EcoRI, and ligated into vectors that are digested with the same enzymes, to create the expression vectors pT7-sTRAILa, pET-sTRAILa, pT7-sTRAILaHis, and pET-sTRAILHis.

The TRAIL receptor agonist DNAs can also be sub-cloned into mammalian expression vector pCEP4 or pcDNA3.1, along with a secretion signal peptide. Expressed proteins are secreted into the culture medium, and the secretion signal peptide is removed during the secretion processes. 5′ primers are flanked with NheI restriction sites and the primers are incorporated into a tetranectin secretion signal peptide, or another secretion signal peptide (e.g., Ig peptide). 3′ primers are flanked with XhoI restriction sites. A 6×His tag is optionally incorporated into the 3′ primers. PCR products are digested with NheI/XhoI, and ligated into the vectors that are digested with the same enzymes, to create expression vectors pCEP4-TRAILa, pcDNA-TRAILa, pCEP4-TRAILaHis, and pcDNA-TRAILaHis.

### Example 22

### Expression and Purification of TRAIL Receptor Agonists from Bacteria

Bacterial expression constructs are transformed into bacterial strain BL21(DE3) (Invitrogen). A single colony on a fresh plate is inoculated into 100 mL of 2xYT medium in a shaker flask. The flask is incubated in a shaker rotating at 250 rpm at 37° C. for 12 h or overnight. Overnight culture (50 mL) is used to inoculate 1 L of 2xYT in a 4 L shaker flask. Bacteria are cultured in the flask to an OD₆₀₀ of about 0.7, at which time IPTG is added to the culture to a final concentration of 1 mM. After a 4 h induction, bacterial pellets are collected by centrifugation and saved for subsequent protein purification.

Bacterial fermentation is performed under fed-batch conditions in a 10-liter fermentor. One liter of complex fermentation medium contains 5 g of yeast extract, 20 g of tryptone, 0.5 g of NaCl, 4.25 g of KH₂PO₄, 4.25 g of K₂HPO₄.3H₂O, 8 g of glucose, 2 g of MgSO₄.7H₂O, and 3 mL of trace metal solution (2.7% FeCl₃.6H₂O/0.2% ZnCl₂.4H₂O/0.2% CoCl₂.6H₂O/0.15% Na₂MoO₄.2H₂O/0.1% CaCl₂.2H₂O/0.1% CuCl₂/0.05% H₃BO₃/3.7% HCl). The fermentor is inoculated with an overnight culture (5% vol/vol) and grown at constant operating conditions at pH 6.9 (controlled with ammonium hydroxide and phosphoric acid) and at 30° C. The airflow rate and agitation are varied to maintain a minimum dissolved oxygen level of 40%. The feed (with 40% glucose) is initiated once the glucose level in the culture is below 1 g /L, and the glucose level is maintained at 0.5 g/L for the rest of the fermentation. When the OD₆₀₀ reaches about 60, IPTG is added into the culture to a final concentration of 0.05 mM. Four hours after induction, the cells are harvested. The bacterial pellet is obtained by centrifugation and stored at −80° C. for subsequent protein purification.

Expressed proteins that are soluble, secreted into the periplasm of the bacterial cell, and include an affinity tag (e.g., 6×His tagged proteins) are purified using standard chromatographic methods, such as metal chelation chromatography (e.g., Ni affinity column), anionic/cationic affinity chromatography, size exclusion chromatography, or any combination thereof, which are well known to one skilled in the art.

Expressed proteins can form insoluble inclusion bodies in bacterial cells. These proteins are purified under denaturing conditions in initial purification steps and undergo a subsequent refolding procedure, which can be performed on a purification chromatography column. The bacterial pellets are suspended in a lysis buffer (0.5 M NaCl, 10 mM Tris-HCl, pH 8, and 1 mM EDTA) and sonicated. The inclusion body is recovered by centrifugation, and subsequently dissolved in a binding buffer containing 6M guanidinium chloride, 50 mM Tri-HCl, pH8, and 0.1 M DTT. The solubilized portion is applied to a Ni affinitycolumn. After washing the unbound materials from the column, the proteins are eluted with an elution buffer (6M guanidinium chloride, 50 mM Tris-HCl pH8.0, 10 mM 2-mercaptoethanol, 250 mM imidazole). Isolated proteins are buffer exchanged into the binding buffer, and are re-applied to the Ni⁺ column to remove the denaturing agent. Once loaded onto the column, the proteins are refolded by a linear gradient (0-0.5M NaCl) using 5 C.V. (column volumes) of a buffer that lacks the denaturant (50 mM Tris-HCl pH8.0, 10 mM 2-mercaptoethanol, plus 2 mM CaCl₂). The proteins are eluted with a buffer containing 0.5M NaCl, 50 mM Tris-HCl pH8.0, and 250 mM imidazole. The fusion tags (6×His, CII6His) are cleaved with Factor Xa or Granzyme B, and removed from protein samples by passage through a Ni⁺-NTA affinity column. The proteins are further purified by ion-exchange chromatography on Q-sepharose (GE) using linear gradients (0-0.5M NaCl) over 10 C.V. in a buffer (50 mM Tris-HCl, pH8.0 and 2 mM CaCl₂). Proteins are dialyzed into 1×PBS buffer. Optionally, endotoxin is removed by passing through a Mustang E filter (PALL).

To prepare soluble extracts from bacterial cells for expressed proteins in the periplasm, the bacterial pellets are suspended in a loading buffer (10 mM phosphate buffer pH6.0), and lysed using sonication (or alternatively a French press). After spinning down the insoluble portion in a centrifuge, the soluble extract is applied to an SP FF column (GE). Periplasmic extracts are also prepared by osmotic shock or “soft” sonication. Secreted soluble 6×His tagged proteins are purified by Ni⁺-NTA column as described above. Crude extracts are buffer exchanged into an affinity column loading buffer, and then applied to an SP FF column. After washing with 4 C.V. of loading buffer, the proteins are eluted using a 100% gradient over 8 C.V. with a high salt buffer (10 mM phosphate buffer, 0.5M NaCl, pH6.0). Eluate is filtered by passing through a Mustang E filter to remove endotoxin. The partially purified proteins are buffer exchanged into 10 mM phosphate buffer, pH7.4, and then loaded to a Q FF column. After washing with 7 C.V. with 10 mM phosphate buffer pH 6.0, the proteins are eluted using a 100% gradient over 8 C.V. with a high salt buffer (10 mM phosphate buffer, pH6.0, 0.5M NaCl). Once again endotoxin is removed by passing through a Mustang E filter.

### Example 23

### Expression and Purification of TRAIL Receptor Agonists from Mammalian Cells

Plasmids for each expression construct are prepared using a Qiagen Endofree Maxi Prep Kit. Plasmids are used to transiently transfect HEK293-EBNA cells. Tissue culture supernatants are collected for protein purification 2-4 days after transfection.

For large-scale production, stable cell lines in CHO or PER.C6 cells are developed to overexpress TRAIL receptor agonists. Cells (5×10⁸) are inoculated into 2.5 L of media in a 20 L bioreactor (Wave). Once the cells have doubled, fresh media (1× start volume) is added, and continues to be added as cells double until the final volume reaches 10 L. The cells are cultured for about 10 days until cell viability drops to 20%. The cell culture supernatant is then collected for purification.

Both His-tagged protein purification (by Ni⁺-NTA column) and non-tagged protein purification (by ion exchange chromatography) are employed as detailed above.

### Example 24

### Inhibition of Cancer Cell Proliferation

Human cancer cell lines expressing DR4 and/or DR5 such as COLO205 (colorectal adenocarcinoma), NCI-H2122 (non-small cell lung cancer), MIA PaCa-2 (pancreatic carcinoma), ACHN (renal cell carcinoma), WM793B (melanoma) and U266B1 (lymphoma) (all purchased from American Type Tissue Collection (Manassas, Va.)) are cultured under the appropriate condition for each cell line and seeded at cell densities of 5,000-20,000 cells/well (as determined appropriate by growth curve for each cancer cell line). DR4/5 agonistic molecules are added at concentrations ranging from 0.0001-100 μg/mL. Optionally DR4/DR5 agonists are combined with therapeutic methods, including chemotherapeutics (e.g., bortezomib) or cells that are pre-sensitized by radiation, to generate a synergistic effect that upregulates DR4 or DR5 or alters caspase activity. The number of viable cells is assessed after 24 and 48 h using “CellTiter 96®AQᵤₑₒᵤₛ One Solution Cell Proliferation Assay” (Promega) according to the manufacturer's instructions, and the IC50 concentrations for the DR4/DR5 agonists are determined.

### Example 25

### Agonist Molecule Assessment in Tumor Xenograft Models

Cancer cell lines (e.g. HCT-116, SW620, COLO205) are injected s.c into Balb/c nude or SCID mice. Tumor length and width is measured twice a week using a caliper. Once the tumor reaches 250 mm³ in size, mice will be randomized and treated i.v. or s.c. with 10-100 mg/kg DR4 or DR5 agonist. Treatment can be combined with other therapeutics such as chemotherapeutics (e.g. irinotecan, bortezomib, or 5FU) or radiation treatment. Tumor size is observed for 30 days unless tumor size reaches 1500 mm³ in which case mice have to be sacrificed.

### Example 26

### Internalization of DR4 Specific Binding Clones with Agonist Activity

Bacterially expressed DR4 ATRIMERS™ were tested for cell internalization following the protocol described below. The physiological ligand TRAIL/Apo2 was used as a positive control and the human WT tetranectin or the H4E (non Trail-R binding clone) ATRIMERS™ were used as a negative control. The DR4 binding proteins including the negative control were conjugated to Alexa Fluor-488 through their amine groups (green fluorescence) following Molecular Probes protocol. Maintenance of proper DR4 binding of ATRIMERS™ or TRAIL after conjugation was verified by Biacore. The H2122 and/or Colo 205 cells (expressing DR4 and DR5) were plated 1 day before the experiment on 12 well tissue culture plates containing a coverslip (poly-D-lysine coated).

The following day the cultures were incubated on ice for 30 min to slow down the metabolism and stop the internalization process. Then the labeled ATRIMERS™ and Trail were added to the cultures at a concentration of 3 μg/well. Cultures were incubated on ice for 45 more minutes to allow the protein to bind to the receptor on the membrane.

After washing the cells to remove unbound conjugated proteins the coverslips were placed in a plate with 2 ml media at 37° C., except for one coverslip from each labeled protein that was immediately fixed with paraformaldehyde (1% in PBS) to analyze binding to the membrane. Cells were incubated with ATRIMERS™ or TRAIL at 37° C. for various times (5 to 60 min) to measure internalization. At the end of the incubations the cells were fixed with 1% paraformaldehyde.

After fixation, membranes were stained using cholera toxin-B conjugated to Alexa-fluor 647 (red) for 20 min and the nucleus was stained with DAPI or Hoechst (blue). (Note: Colera toxin B binds to lipid rafts on the membrane). Finally the coverslips were mounted on slides using the mounting media ProLong Gold, and then sealed with nail polish and analyzed in a Confocal Microscope. ATRIMERS™ tested so far show a strong correlation between the degree of internalization and strength of agonist activity (Table 16).

TABLE 16
Relative Internalization of DR4 specific ATRIMERS ™

| | Internalization | Cells tested |
|---|---|---|
| TRAIL | Yes | H2122 and Colo 205 |
| 56p53PH4E (Negative | No | H2122 |
| control) | | |
| hTN4 (Negative control) | No | H2122 |
| 14p42P3B8 | No | H2122 |
| 142p5P1E11 | No | H2122 |
| 119p94P1B5 | Yes (++) | H2122 |
| 71p88P1B3 | Yes (+) | Colo 205 |
| 119p83P1H4 | Yes (++++) | Colo 205 |

### Example 27

### Activation of Caspases by DR5 and DR4 Agonistic Molecules in Cancer Cell Lines

Human cancer cell lines expressing DR4 and/or DR5 such as COLO205 (colorectal adenocarcinoma), NCI-H2122 (non-small cell lung cancer), MIA PaCa-2 (pancreatic carcinoma), ACHN (renal cell carcinoma), WM793B (melanoma) and U266B1 (lymphoma) (all purchased from American Type Tissue Collection (Mannasas, Va.)) are cultured under the appropriate condition for each cell line and seeded at cell densities of 5,000-20,000 cells/well (as determined appropriate by growth curve for each cancer cell line). DR4/5 agonistic molecules are added at concentrations ranging from 0.0001-100 μg/mL. DR4/DR5 agonists can be combined with other therapies such as chemotherapeutics (e.g., bortezomib) or cells that are pre-sensitized by radiation to determine whether such a combination has a synergistic effect on up-regulation of DR4 or DR5 or altering caspase activity. Caspase activity is determined at various timepoints using the “APO-ONE Caspase assay” (Promega) according to the manufacturers instruction.

Further analysis by Western Blot is performed by incubating 2×10⁶ tumor cells as described above. Subsequent cell lysates are prepared for Western Blot. Proteins are separated by SDS-PAGE and transferred to nitrocellulose membranes. The filters are incubated with antibodies that recognize the pro and cleaved forms of the apoptotic proteins PARP, caspase 3, caspase 8, caspase 9, bid and actin. The bands corresponding to specific proteins are detected by HRP-conjugated secondary antibodies and enhanced chemiluminescence.

### Example 28

### Affinity Maturation of TRAIL Receptor Agonists Assisted by in Silico Modeling

In silico modeling is used to affinity mature TRAIL receptor agonists that are identified from the CTLD phage display library screening. Agonist homology models are built based on the known tetranectin 3D structures. Loop conformations of homology models of agonists are refined and optimized using LOOPER (DS2.1, Accelrys) and their related algorithms. This process includes three basic steps: 1. Construction of a set of possible loop conformers with optimized interactions of loop backbone with the rest of the protein; 2. Building and structural optimization of loop side chains and energy minimization applied to all loop atoms; 3. Final scoring and ranking the retained variants of loop conformers. Potential binding regions or epitopes located on the DR4/DR5 extracellular domain are identified for the agonists using a combination of manual and molecular dynamics-based docking. The binding domains are further confirmed by performing binding assays using deletion or point mutations of DR4/DR5 extracellular domain(s) and the agonists. Amino acid residues (or sequences) that are involved in determining binding specificity are defined on both DR4/DR5 and TRAIL CTLD agonists. A combination of random mutations at various target positions is screened using structure-based computation to determine the compatibility with the structure template. Based on the analysis of apparent packing defects, residues are selected for mutagenesis to construct a library for phage display.

The 3D models of TRAIL receptor agonist peptides and DR4/DR5 can be used as a reference to refine the peptide-grafted CTLD and DR4/DR5 modeling. When TRAIL receptor agonist peptides are grafted into CTLD loops, loop conformations are optimized and re-surfaced to match agonist peptides/DR4/DR5 binding by changing the flanking and surrounding amino acid residues using in silico modeling. Peptide grafted CTLD agonist homology models are built based on the known tetranectin 3D structures. Loop conformations of homology models of agonists are refined and optimized using LOOPER (DS2.1, Accelrys) and their related algorithms as described above. A combination of random mutations at various target positions is screened by structure-based computation for their compatibility with the structure template. Based on analysis of apparent packing defects, amino acid residues flanking and surrounding peptides are selected for mutagenesis to construct a library for phage display.

The above examples do not limit the scope of variation that can be generated in these libraries. Other libraries can be generated in which varying numbers of random or more targeted amino acids are used to replace existing amino acids, and different combinations of loops can be utilized. In addition, other mutations and methods of generating mutations, such as random PCR mutagenesis, can be utilized to provide diverse libraries that can be subjected to panning.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention.

The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. The disclosures of all references and publications cited herein are expressly incorporated by reference in their entireties to the same extent as if each were incorporated by reference individually.

TABLE 16
TRAIL-Related Sequences

| | | |
|---|---|---|
| Sequence | | SEQ ID |
| Description | Sequence | NO: |

| | | |
|---|---|---|
| Human TRAIL | MAMMEVQGGP SLGQTCVLIV IFTVLLQSLC VAVTYVYFTN | 136 |
| GenBank Acc. | ELKQMQDKYS KSGIACFLKE DDSYWDPNDE ESMNSPCWQV | |
| P50591 | KWQLRQLVRK MILRTSEETI STVQEKQQNI SPLVRERGPQ | |
| 281 AA | RVAAHITGTR GRSNTLSSPN SKNEKALGRK INSWESSRSG | |
| | HSFLSNLHLR NGELVIHEKG FYYIYSQTYF RFQEEIKENT | |
| | KNDKQMVQYI YKYTSYPDPI LLMKSARNSC WSKDAEYGLY | |
| | SIYQGGIFEL KENDRIFVSV TNEHLIDMDH EASFFGAFLV G | |
| DR4; TRAIL-R1 | MAPPPARVHL GAFLAVTPNP GSAASGTEAA AATPSKVWGS | 137 |
| GenBank Acc. | SAGRIEPRGG GRGALPTSMG QHGPSARARA GRAPGPRPAR | |
| O00220 | EASPRLRVHK TFKFVVVGVL LQVVPSSAAT IKLHDQSIGT | |
| 468 AA | QQWEHSPLGE LCPPGSHRSE HPGACNRCTE GVGYTNASNN | |
| | LFACLPCTAC KSDEEERSPC TTTRNTACQC KPGTFRNDNS | |
| | AEMCRKCSRG CPRGMVKVKD CTPWSDIECV HKESGNGHNI | |
| | WVILVVTLVV PLLLVAVLIV CCCIGSGCGG DPKCMDRVCF | |
| | WRLGLLRGPG AEDNAHNEIL SNADSLSTFV SEQQMESQEP | |
| | ADLTGVTVQS PGEAQCLLGP AEAEGSQRRR LLVPANGADP | |
| | TETLMLFFDK FANIVPFDSW DQLMRQLDLT KNEIDVVRAG | |
| | TAGPGDALYA MLMKWVNKTG RNASIHTLLD ALERMEERHA | |
| | KEKIQDLLVD SGKFIYLEDG TGSAVSLE | |
| DR5; TRAIL-R2 | MEQRGQNAPA ASGARKRHGP GPREARGARP GPRVPKTLVL | 138 |
| GenBank Acc. | VVAAVLLLVS AESALITQQD LAPQQRAAPQ QKRSSPSEGL | |
| O14763 | CPPGHHISED GRDCISCKYG QDYSTHWNDL LFCLRCTRCD | |
| 440 AA | SGEVELSPCT TTRNTVCQCE EGTFREEDSP EMCRKCRTGC | |
| | PRGMVKVGDC TPWSDIECVH KESGTKHSGE APAVEETVTS | |
| | SPGTPASPCS LSGIIIGVTV AAVVLIVAVF VCKSLLWKKV | |
| | LPYLKGICSG GGGDPERVDR SSQRPGAEDN VLNEIVSILQ | |
| | PTQVPEQEME VQEPAEPTGV NMLSPGESEH LLEPAEAERS | |
| | QRRRLLVPAN EGDPTETLRQ CFDDFADLVP FDSWEPLMRK | |
| | LGLMDNEIKV AKAEAAGHRD TLYTMLIKWV NKTGRDASVH | |
| | TLLDALETLG ERLAKQKIED HLLSSGKFMY LEGNADSAMS | |
| TRAIL-R3 | MARIPKTLKF VVVIVAVLLP VLAYSATTAR QEEVPQQTVA | 139 |
| GenBank Acc. | PQQQRHSFKG EECPAGSHRS EHTGACNPCT EGVDYTNASN | |
| O14798 | NEPSCFPCTV CKSDQKHKSS CTMTRDTVCQ CKEGTFRNEN | |
| 259 AA | SPEMCRKCSR CPSGEVQVSN CTSWDDIQCV EEFGANATVE | |
| | TPAAEETMNT SPGTPAPAAE ETMNTSPGTP APAAEETMTT | |
| | SPGTPAPAAE ETMTTSPGTP APAAEETMTT SPGTPASSHY | |
| | LSCTIVGIIV LIVLLIVFV | |
| TRAIL-R4 | MGLWGQSVPT ASSARAGRYP GARTASGTRP WLLDPKILKF | 140 |
| GenBank Acc. | VVFIVAVLLP VRVDSATIPR QDEVPQQTVA PQQQRRSLKE | |
| Q9UBN6 | EECPAGSHRS EYTGACNPCT EGVDYTIASN NLPSCLLCTV | |
| 386 AA | CKSGQTNKSS CTTTRDTVCQ CEKGSFQDKN SPEMCRTCRT | |
| | GCPRGMVKVS NCTPRSDIKC KNESAASSTG KTPAAEETVT | |
| | TILGMLASPY HYLIIIVVLV IILAVVVVGF SCRKKFISYL | |
| | KGICSGGGGG PERVHRVLFR RRSCPSRVPG AEDNARNETL | |
| | SNRYLQPTQV SEQEIQGQEL AELTGVTVES PEEPQRLLEQ | |
| | AEAEGCQRRR LLVPVNDADS ADISTLLDAS ATLEEGHAKE | |
| | TIQDQLVGSE KLFYEEDEAG SATSCL | |
| OPG | MNNLLCCALV FLDISIKWTT QETFPPKYLH YDEETSHQLL | 141 |
| GenBank Acc. | CDKCPPGTYL KQHCTAKWKT VCAPCPDHYY TDSWHTSDEC | |
| NP_002537 | LYCSPVCKEL QYVKQECNRT HNRVCECKEG RYLEIEFCLK | |
| 401 AA | HRSCPPGFGV VQAGTPERNT VCKRCPDGFF SNETSSKAPC | |
| | RKHTNCSVFG LLLTQKGNAT HDNICSGNSE STQKCGIDVT | |
| | LCEEAFFRFA VPTKFTPNWL SVLVDNLPGT KVNAESVERI | |
| | KRQHSSQEQT FQLLKLWKHQ NKDQDIVKKI IQDIDLCENS | |
| | VQRHIGHANL TFEQLRSLME SLPGKKVGAE DIEKTIKACK | |
| | PSDQILKLLS LWRIKNGDQD TLKGLMHALK HSKTYHFPKT | |
| | VTQSLKKTIR FLHSFTMYKL YQKLFLEMIG NQVQSVKISC L | |

TABLE 17
Other Death Receptor Sequence Information

| Protein | References |
|---|---|
| Fn14 | Genbank U42386 [Mus musculus fibroblast |
| FIN14 | growth factor inducible gene 14 (FIN14) |
| (Fibroblast | mRNA, complete cds] He et al. (2009), |
| growth factor | “Solution structure of the cysteine-rich |
| inducible 14) | domain in Fn14, a member of the tumor |
| | necrosis factor receptor superfamily.” |
| | Protein Sci. 18(3): 650-6. |
| FAS | Genbank NM_000043 [Homo sapiens Fas (TNF |
| (TNF receptor | receptor superfamily, member 6) (FAS), |
| superfamily, | transcript variant 1, mRNA] |
| member 6) | Lundin et al. (2004), “CD4+ T cells kill |
| | Id+ B-lymphoma cells: FasLigand-Fas |
| | interaction is dominant in vitro but is |
| | redundant in vivo.” Cancer Immunol. |
| | Immunother. 53(12): 1135-45. |
| LIGHT | Zhai et al. (1998). “LIGHT, a novel |
| (Lymphotoxin-like | ligand for lymphotoxin beta receptor and |
| Inducible protein | TR2/HVEM induces apoptosis and |
| that competes with | suppresses in vivo tumor formation via |
| Glycoprotein D for | gene transfer.” J. Clin. Invest. 102: |
| Herpesvirus | 1142-1151. |
| entry on T cells) | |

TABLE 18
TAS and TAA sequence information:

| Protein | References |
|---|---|
| AFP | Genbank NM_001134 [Homo sapiens alpha-fetoprotein |
| alfafetoprotein | (AFP), mRNA] |
| alphafetoprotein | Williams et al. (1977), “Tumor-associated antigen levels |
| alpha-fetoprotein | (carcinoembryonic antigen, human chorionic gonadotropin, |
| | and alpha-fetoprotein) antedating the diagnosis of cancer in |
| | the Framingham study.” J. Natl. Cancer Inst. 58(6): 1547-51. |
| CEA | Genbank M29540 [Human carcinoembryonic antigen |
| carcinoembryonic | mRNA (CEA), complete cds] |
| antigen | Williams et al. (1977), “Tumor-associated antigen levels |
| | (carcinoembryonic antigen, human chorionic gonadotropin, |
| | and alpha-fetoprotein) antedating the diagnosis of cancer in |
| | the Framingham study.” J. Natl. Cancer Inst. 58(6): 1547-51. |
| CA-125 | Genbank NM_024690 [Homo sapiens mucin 16, cell |
| cancer antigen 125 | surface associated (MUC16), mRNA] |
| carbohydrate antigen 125 | Boivin et al. (2009), “CA125 (MUC16) tumor antigen |
| also known as | selectively modulates the sensitivity of ovarian cancer cells |
| MUC16 | to genotoxic drug-induced apoptosis.” Gynecol. Oncol., |
| mucin 16 | Sep. 9, Epub ahead of print. |
| MUC1 | Genbank BC120974 [Homo sapiens mucin 1, cell surface |
| mucin 1 also known as | associated, mRNA (cDNA clone MGC: 149467 |
| epithelial tumor antigen | IMAGE: 40115473), complete cds] |
| | Acres and Limacher (2005), “MUC1 as a target antigen for |
| | cancer immunotherapy.” Expert Rev. Vaccines 4(4): 493-502. |
| glypican 3 | Genbank BC035972 [Homo sapiens glypican 3, mRNA |
| | (cDNA clone MGC: 32604 IMAGE: 4603748), complete cds] |
| | Nakatsura and Nishimura (2005), “Usefulness of the novel |
| | oncofetal antigen glypican-3 for diagnosis of |
| | hepatocellular carcinoma and melanoma.” BioDrugs 19(2): 71-7. |
| TAG-72 | Lottich et al. (1985), “Tumor-associated antigen TAG-72: |
| tumor-associated | correlation of expression in primary and metastatic breast |
| glycoprotein 72 | carcinoma lesions.” Breast Cancer Res. Treat. 6(1): 49-56. |
| tyrosinase | Genbank BC027179 [Homo sapiens tyrosinase |
| | (oculocutaneous albinism IA), mRNA (cDNA clone |
| | MGC: 9191 IMAGE: 3923096), complete cds] |
| MAA | Genbank BC144138 [Homo sapiens melanoma associated |
| melanoma-associated antigen | antigen (mutated) 1, mRNA (cDNA clone MGC: 177675 |
| | IMAGE: 9052658), complete cds] |
| | Chee et al. (1976), “Production of melanoma-associated |
| | antigen(s) by a defined malignant melanoma cell strain |
| | grown in chemically defined medium.” Cancer Res. 36(4): |
| | 1503-9. |
| MART-1 | Genbank BC014423 [Homo sapiens melan-A, mRNA |
| melanoma antigen recognized by | (cDNA clone MGC: 20165 IMAGE: 4639927), complete cds] |
| T-cells 1 | Du et al. (2003), “MLANA/MART1 and |
| also known as | SILV/PMEL17/GP100 are transcriptionally regulated by |
| MLANA | MITF in melanocytes and melanoma.” Am. J. Pathol. |
| melan-A | 163(1): 333-43. |
| gp100 | Adema et al. (1994), “Molecular characterization of the |
| | melanocyte lineage-specific antigen gp100.” J. Biol. Chem. |
| | 269(31): 20126-33. |
| | Zhai et al. (1996), “Antigen-specific tumor vaccines. |
| | Development and characterization of recombinant |
| | adenoviruses encoding MART1 or gp100 for cancer |
| | therapy.” J. Immunol. 156(2): 700-10. |
| TRP1 | Genbank AF001295 [Homo sapiens tyrosinase related |
| tyrosinase-related protein 1 | protein 1 (TYRP1) gene, complete cds] |
| | Wang and Rosenberg (1996), “Human tumor antigens |
| | recognized by T lymphocytes: implications for cancer |
| | therapy.” J. Leukoc. Biol. 60(3): 296-309. |
| TRP2 | Genbank L18967 [Homo sapiens TRP-2/dopachrome |
| tyrosinase-related protein 2 | tautomerase (Tyrp-2) mRNA, complete cds] |
| dopachrome tautomerase | Wang et al. (1996), “Identification of TRP-2 as a human |
| | tumor antigen recognized by cytotoxic T lymphocytes.” J. |
| | Exp. Med. 184(6): 2207-16. |
| MSH1 | Genbank NP_011988 [DNA-binding protein of the |
| Note: in yeast only-this protein is | mitochondria involved in repair of mitochondrial DNA, |
| not present in humans. | has ATPase activity and binds to DNA mismatches; has |
| | homology to E. coli MutS; transcription is induced during |
| | meiosis; Msh1p [Saccharomyces cerevisiae]] |
| | Foury et al. (2004), “Mitochondrial DNA mutators.” Cell. |
| | Mol. Life Sci. 61(22): 2799-811. |
| MAGE-1 | Genbank NP_004979 [melanoma antigen family A, 1 |
| MAGEA1 | [Homo sapiens]] |
| melanoma antigen family A 1 | Zakut et al. (1993), “Differential expression of MAGE-1, -2, |
| melanoma-associated antigen 1 | and -3 messenger RNA in transformed and normal |
| | human cell lines.” Cancer Res. 53(1): 5-8. |
| | Eichmuller et al. (2002), “mRNA expression of tumor- |
| | associated antigens in melanoma tissues and cell lines.” |
| | Exp. Dermatol. 11(4): 292-301. |
| MAGE-2 | Genbank L18920 [Human MAGE-2 gene exons 1-4, |
| MAGEA2 | complete cds] |
| melanoma antigen family A 2 | Zakut et al. (1993), “Differential expression of MAGE-1, -2, |
| melanoma-associated antigen 2 | and -3 messenger RNA in transformed and normal |
| | human cell lines.” Cancer Res. 53(1): 5-8. |
| MAGE-3 | Genbank U03735 [Human MAGE-3 antigen (MAGE-3) |
| MAGEA3 | gene, complete cds] |
| melanoma antigen family A 3 | Zakut et al. (1993), “Differential expression of MAGE-1, -2, |
| melanoma-associated antigen 3 | and -3 messenger RNA in transformed and normal |
| | human cell lines.” Cancer Res. 53(1): 5-8. |
| MAGE-12 | Genbank NP_005358 [melanoma antigen family A, 12 |
| MAGEA12 | [Homo sapiens]] |
| melanoma antigen family A 12 | Gibbs et al. (2000), “MAGE-12 and MAGE-6 are |
| melanoma-associated antigen 12 | frequently expressed in malignant melanoma.” Melanoma |
| | Res. 10(3): 259-64. |
| RAGE-1 | Genbank BC053536 [Homo sapiens renal tumor antigen, |
| renal tumor antigen 1 | mRNA (cDNA clone MGC: 61453 IMAGE: 5175851), |
| | complete cds] |
| | Eichmuller et al. (2002), “mRNA expression of tumor- |
| | associated antigens in melanoma tissues and cell lines.” |
| | Exp. Dermatol. 11(4): 292-301. |
| GAGE-1 | Genbank U19141 [Human GAGE-1 protein mRNA, |
| G antigen 1 | complete cds] |
| | Eichmuller et al. (2002), “mRNA expression of tumor- |
| | associated antigens in melanoma tissues and cell lines.” |
| | Exp. Dermatol. 11(4): 292-301. |
| | De Backer et al. (1999), “Characterization of the GAGE |
| | genes that are expressed in various human cancers and in |
| | normal testis.” Cancer Res. 59(13): 3157-65. |
| GAGE-2 | Genbank U19143 [Human GAGE-2 protein mRNA, |
| G antigen 2 | complete cds] |
| | De Backer et al. (1999), “Characterization of the GAGE |
| | genes that are expressed in various human cancers and in |
| | normal testis.” Cancer Res. 59(13): 3157-65. |
| BAGE | Genbank BC107038 [Homo sapiens B melanoma antigen, |
| B melanoma antigen | mRNA (cDNA clone MGC: 129548 IMAGE: 40002186), |
| | complete cds] |
| | Boel et al. (1995), “BAGE: a new gene encoding an |
| | antigen recognized on human melanomas by cytolytic T |
| | lymphocytes.” Immunity 2(2): 167-75. |
| NY-ESO-1 | Genbank BC130362 [Homo sapiens cancer/testis antigen |
| also known as | 1B, mRNA (cDNA clone MGC: 163234 |
| cancer/testis antigen 1B | IMAGE: 40146393), complete cds] |
| | Schultz-Thater et al. (2000), “NY-ESO-1 tumour |
| | associated antigen is a cytoplasmic protein detectable by |
| | specific monoclonal antibodies in cell lines and clinical |
| | specimens.” Br. J. Cancer 8(2): 204-8. |
| beta-catenin | Genbank NM_001098209 [Homo sapiens catenin |
| | (cadherin-associated protein), beta 1, 88 kDa (CTNNB1), |
| | mRNA] |
| CDCP-1 | Genbank BC021099 [Homo sapiens CUB domain |
| CUB domain containing protein 1 | containing protein 1, mRNA (cDNA clone |
| | IMAGE: 4590554), complete cds] |
| | Wortmann et al. (2009), “The cell surface glycoprotein |
| | CDCP1 in cancer--insights, opportunities, and challenges.” |
| | IUBMB Life 61(7): 723-30. |
| CDC-27 | Genbank BC011656 [Homo sapiens cell division cycle 27 |
| cell division cycle 27 homolog | homolog (S. cerevisiae), mRNA (cDNA clone MGC: 12709 |
| | IMAGE: 4301175), complete cds] |
| | Wang et al. (1999), “Cloning genes encoding MHC class |
| | II-restricted antigens: mutated CDC27 as a tumor antigen.” |
| | Science 284: 1351-4. |
| SART-1 | Genbank BC001058 [Homo sapiens squamous cell |
| squamous cell carcinoma | carcinoma antigen recognized by T cells, mRNA (cDNA |
| antigen recognized by T-cells | clone MGC: 2038 IMAGE: 3504745), complete cds] |
| | Hosokawa et al. (2005), “Cell cycle arrest and apoptosis |
| | induced by SART-1 gene transduction.” Anticancer Res. |
| | 25(3B): 1983-90. |
| EpCAM | Genbank BC014785 [Homo sapiens epithelial cell |
| epithelial cell adhesion molecule | adhesion molecule, mRNA (cDNA clone MGC: 9040 |
| | IMAGE: 3861826), complete cds] |
| | Munz et al. (2009), “The emerging role of EpCAM in |
| | cancer and stem cell signaling.” Cancer Res. 69(14): 5627-9. |
| CD20 | Genbank BC002807 [Homo sapiens membrane-spanning |
| also known as | 4-domains, subfamily A, member 1, mRNA (cDNA clone |
| membrane-spanning 4-domains, | MGC: 3969 IMAGE: 3634040), complete cds.] |
| subfamily A, member 1 | Tedder et al. (1988), “Isolation and structure of a cDNA |
| | encoding the B1 (CD20) cell-surface antigen of human B |
| | lymphocytes.” Proc. Natl. Acad. Sci. USA 85(1): 208-12. |
| CD23 | Genbank BC062591 [Homo sapiens Fc fragment of IgE, |
| also known as | low affinity II, receptor for (CD23), mRNA (cDNA clone |
| receptor for Fc fragment of IgE, | MGC: 74689 IMAGE: 5216918), complete cds] |
| low affinity II | Bund et al. (2007), “CD23 is recognized as tumor- |
| | associated antigen (TAA) in B-CLL by CD8+ autologous |
| | T lymphocytes.” Exp. Hematol. 35(6): 920-30. |
| CD33 | Genbank BC028152 [Homo sapiens CD33 molecule, |
| | mRNA (cDNA clone MGC: 40026 IMAGE: 5217182), |
| | complete cds] |
| | Peiper et al. (1988), “Molecular cloning, expression, and |
| | chromosomal localization of a human gene encoding the |
| | CD33 myeloid differentiation antigen.” Blood 72(1): 314-21. |
| EGFR | Genbank NM_005228 [Homo sapiens epidermal growth |
| epidermal growth factor | factor receptor (erythroblastic leukemia viral (v-erb-b) |
| receptor | oncogene homolog, avian) (EGFR), transcript variant 1, |
| | mRNA] |
| | Kordek et al. (1994), “Expression of a p53-protein, |
| | epidermal growth factor receptor (EGFR) and proliferating |
| | cell antigens in human gliomas.” Folia Neuropathol. 32(4): 227-8. |
| HER-2 | Genbank NM_001005862 [Homo sapiens v-erb-b2 |
| also known as | erythroblastic leukemia viral oncogene homolog 2, |
| v-erb-b2 erythroblastic leukemia | neuro/glioblastoma derived oncogene homolog (avian) |
| viral oncogene homolog 2, | (ERBB2), transcript variant 2, mRNA] |
| neuro/glioblastoma derived | Neubauer et al. (2008), “Changes in tumour biological |
| oncogene homolog (avian) | markers during primary systemic chemotherapy (PST).” |
| | Anticancer Res. 38(3B): 1797-804. |
| BTA-1 | |
| breast tumor-associated antigen 1 | |
| BTA-2 | |
| breast tumor-associated antigen 2 | |
| RCAS1 | Genbank BC022506 [Homo sapiens estrogen receptor |
| receptor-binding cancer antigen | binding site associated, antigen, 9, mRNA (cDNA clone |
| expressed on SiSo cells | MGC: 26497 IMAGE: 4815654), complete cds] |
| also known as | Giaginis et al. (2009), “Receptor-binding cancer antigen |
| estrogen receptor binding side | expressed on SiSo cells (RCAS1): a novel biomarker in the |
| associated antigen 9 | diagnosis and prognosis of human neoplasia.” Histol. |
| | Histopathol. 24(6): 761-76. |
| PLAC1 | Genbank BC022335 [Homo sapiens placenta-specific 1, |
| placenta-specific 1 | mRNA (cDNA clone MGC: 22788 IMAGE: 4769552), |
| | complete cds] |
| | Dong et al. (2008), “Plac1 is a tumor-specific antigen |
| | capable of eliciting spontaneous antibody responses in |
| | human cancer patients.” Int. J. Cancer 122(9): 2038-43. |
| syndecan | Genbank BC008765 [Homo sapiens syndecan 1, mRNA |
| | (cDNA clone MGC: 1622 IMAGE: 3347793), complete |
| | cds] |
| | Sun et al. (1997), “Large scale and clinical grade |
| | purification of syndecan-1+ malignant plasma cells.” J. |
| | Immunol. Methods 205(1): 73-9. |
| gp250 | Genbank BC137171 [Homo sapiens sortilin-related |
| also known as | receptor, L(DLR class) A repeats-containing, mRNA |
| sortilin-related receptor, L(DLR | (cDNA clone MGC: 168791 IMAGE: 9021168), complete |
| class) A repeats-containing | cds] |

### BIBLIOGRAPHY

All published patent applications identified herein are incorporated by reference in their entirety.
Agnew, Chem. Intl. Ed. Engl., 33: 183-186 (1994)
Ashkenazi, et al. J Clin Invest.; 104(2):155-62 (July 1999).
Chemotherapy Service Ed., M. C. Perry, Williams & Wilkins, Baltimore, Md. (1992)
Ausubel et al., Current Protocols in Molecular Biology (eds., Green Publishers Inc. and Wiley and Sons 1994
Degli-Esposti et al., Immunity, 7(6):813-820 (December 1997)
Degli-Esposti et al., J. Exp. Med., 186(7):1165-1170 (Oct. 6, 1997)
Janeway, Nature, 341(6242): 482-3 (Oct. 12, 1989)
Jin et al, Cancer Res., 15;64(14):4900-5 (July 2004).
Langer et al., J. Biomed. Mater. Res., 15: 167-277 (1981)
Langer, Chem. Tech., 12: 98-105 (1982)
Marsters et al., Curr. Biol., 7:1003-1006 (1997)
McFarlane et al., J. Biol. Chem., 272:25417-25420 (1997)
Mongkolsapaya et al., J. Immunol., 160:3-6 (1998)
Mordenti et al., Pharmaceut. Res., 8:1351 (1991)
Neame, et al., Protein Sci., 1(1):161-8 (1992)
Neame, P. J. and Boynton, R. E., Protein Soc. Symposium, (Meeting date 1995; 9th Meeting: Tech. Prot. Chem. VII). Proceedings pp. 401-407 (Ed., Marshak, D. R.; Publisher: Academic, San Diego, Calif.) (1996).
Offner et al., Science, 251: 430-432 (1991)
Pan et al., FEBS Letters, 424:41-45 (1998)
Pan et al., Science, 276:111-113 (1997)
Pan et al., Science, 277:815-818 (1997)
Remington's Pharmaceutical Sciences, 16th edition, Osol, A. ed. (1980)
S. G. Hymowitz, et. al., Mol. Cell. 1999 Oct.; 4(4):563-71)
Sambrook, et al. Molecular Cloning: A Laboratory Manual. Cold Spring Harbor Laboratory Press, Cold Spring Harbor, N.Y. (1989)
Schneider et al., FEBS Letters, 416:329-334 (1997)
Screaton et al., Curr. Biol., 7:693-696 (1997)
Sheridan et al., Science, 277:818-821 (1997)
Sidman et al., Biopolymers, 22: 547-556 (1983)
Sorensen et al., Gene, 152: 243-245 (1995)
Cha et. al., J Biol. Chem., 275(40):31171-7 (Oct. 6, 2000).
Murakami et al., The Molecular Basis of Cancer, Mendelsohn and Israel, eds., Chapter 1, entitled “Cell cycle regulation, oncogenes, and antineoplastic drugs” by (WB Saunders: Philadelphia, pg. 13 (1995).
Walczak et al., EMBO J., 16:5386-5387 (1997)
Wu et al., Nature Genetics, 17:141-143 (1997)
Dunn, I. S., Curr Opin Biotechnol., 7(5):547-53 (1996)
Griffiths and Duncan, Curr Opin Biotechnol. 9(1):102-8 (1998)
Marks et al., J Biol. Chem. 267(23):16007-10 (1992)
Mikawa et al. J Mol Biol., 262(1):21-30 (1996)
Ernst et al., Eur J. Biochem., 267(13):4033-9 (2000)
Benhar et al., J Mol Biol., 301(4):893-904 (2000)
Boder and Wittrup, Nat. Biotechnol., 15:553-7 (1997)
Whitehorn et al., Biotechnology, (N Y), 11:1215-9 (1995)
Schaffitzel et al., J Immunol Methods, 231(1-2):119-35 (1999)
Gates et al., J Mol Biol., 255(3):373-86 (1996).

What is claimed is:

1. A TRAIL death receptor agonist comprising a polypeptide that binds to TRAIL death receptor DR4 and comprises a C-Type Lectin Like Domain (CLTD) comprising one of the following combinations of sequences in loops 1 and 4:

| | Loop 1 | | Loop 4 |
|---|---|---|---|
| | SEQ | | SEQ |
| Loop 1 | ID NO | Loop 4 | ID NO |
| GWLEGSGW | 428 | DGGVQWRWEN | 436 |
| GYMTGVGW | 429 | DGGRSWKWEN | 437 |
| GWMEGVGW | 430 | DGGPPWRWEN | 438 |
| GWLEGSGW | 428 | DGGFPARWEN | 439 |
| GWMDGSGW | 431 | DGGRLWRWEN | 440 |
| GWMAGVGW | 290 | DGGPGLRWEN | 441 |
| GYLAGTGW | 432 | DGGRVLAWEN | 443 |
| GWLAGSGW | 433 | DGGGGWPWEN | 443 |
| GWVAGVGW | 434 | DGGGGWRWEN | 444 |
| GWIEGAGW | 435 | DGGWRSRWEN | 445 |
| GWLEGYGW | 265 | DGGAERAWEN | 446 |
| GWLEGVGW | 261 | DGGWPFSNEN | 315 |

2. The polypeptide of claim 1, wherein the at least one polypeptide that binds to a TRAIL death receptor further comprises one of the following sequences for loop 3:

| Loop 3 SEQ | SEQ ID NO |
|---|---|
| NWGDQRLAQ | 496 |
| NWADERRNQ | 497 |
| NWADKRWLQ | 498 |
| NWKDDRFNQ | 499 |
| NWLDPRMGQ | 500 |
| NWYSDYLNQ | 501 |
| NWHYqKYIQ | 502 |
| NWALDRYNQ | 503 |
| NWGRPELAQ | 504 |
| NWANPSFMQ | 505 |
| NWADERFLQ | 506 |
| NWGRELAQ | 507 |
| NWTQRHSGQ | 451 |
| NWARHINEQ | 452 |
| NWYSWPKLQ | 453 |
| NWGWSARVQ | 457 |
| NWGWMDSKQ | 458 |
| NWWFPTLSQ | 459 |
| NWGDPRWSQ | 545 |
| NWADPKWSQ | 569 |
| NWFHDRFNQ | 570 |

3. The polypeptide of claim 2 wherein Loop 1 is SEQ ID NO: 428 and Loop 4 is SEQ ID NO: 436.

4. The polypeptide of claim 3, wherein the polypeptide does not bind to a TRAIL decoy receptor, wherein the TRAIL decoy receptor is at least one of DcR1, DcR2, and circulating osteoprotegerin (OPG).

5. The polypeptide of claim 1 further comprising a polypeptide that binds to DR5.

6. The polypeptide of claim 1 further comprising a second polypeptide that binds to DR4.

7. A non-natural polypeptide comprising a trimerizing domain and at least one polypeptide according to claim 1, wherein the trimerizing domain comprises a polypeptide of SEQ ID NO: 10 having up to five amino acid substitutions at positions 10, 17, 20, 21, 24, 25, 26, 28, 29, 30, 31, 32, 33, 34, or 35, and wherein three trimerizing domains form a trimeric complex.

8. A non-natural polypeptide comprising a trimerizing domain and at least one polypeptide according to claim 1, wherein the trimerizing domain comprises a trimerizing polypeptide that is derived from a polypeptide selected from the group consisting of hTRAF3 [SEQ ID NO: 2], hMBP [SEQ ID NO: 3], hSPC300 [SEQ ID NO: 4], hNEMO [SEQ ID NO: 5], hcubilin [SEQ ID NO: 6], hThrombospondins [SEQ ID NO: 7], and neck region of human SP-D, [SEQ ID NO: 8], neck region of bovine SP-D [SEQ ID NO: 9], neck region of rat SP-D [SEQ ID NO: 11], neck region of bovine conglutinin: [SEQ ID NO: 12]; neck region of bovine collectin: [SEQ ID NO: 13]; and neck region of human SP-D: [SEQ ID NO: 14].

9. The non-natural polypeptide of claim 8 wherein the trimerizing domain is at least 85% identical to a polypeptide selected from the group consisting of hTRAF3 [SEQ ID NO: 2], hMBP [SEQ ID NO: 3], hSPC300 [SEQ ID NO: 4], hNEMO [SEQ ID NO: 5], hcubilin [SEQ ID NO: 6], hThrombospondins [SEQ ID NO: 7], and neck region of human SP-D, [SEQ ID NO: 8], neck region of bovine SP-D [SEQ ID NO: 9], neck region of rat SP-D [SEQ ID NO: 11], neck region of bovine conglutinin: [SEQ ID NO: 12]; neck region of bovine collectin: [SEQ ID NO: 13]; and neck region of human SP-D: [SEQ ID NO: 14].

10. The polypeptide of claim 7 wherein the polypeptide that binds DR4 is positioned at one of the N-terminus and the C-terminus of the trimerizing domain, and further comprising a polypeptide sequence that binds a tumor-associated antigen (TAA) or tumor-specific antigen (TSA) at the other of the N-terminus and the C-terminus.

11. The polypeptide of claim 10 wherein the polypeptide binds to a tumor-associated antigen (TAA) or tumor-specific antigen (TSA) with at least two times greater affinity than the polypeptide binds to DR4 or DR5.

12. The polypeptide of claim 7 wherein the polypeptide that binds DR4 is positioned at one of the N-terminus and the C-terminus of the trimerizing domain, and further comprising a polypeptide sequence that binds a receptor selected from the group consisting of Fn14, FAS receptor, TNF receptor, and LIGHT receptor, at the other of the N-terminus and the C-terminus.

13. A trimeric complex comprising three polypeptides of claim 7.

14. The trimeric complex of claim 13 wherein the complex further comprises three polypeptide sequences that specifically bind DR5, wherein the sequences can be the same or different.

15. A method of inducing apoptosis in a tumor cell in a patient expressing at least one of DR4 and DR5 comprising contacting the cell with the trimeric complex of claim 13.

16. The method of claim 15 wherein the trimeric complex induces caspase-dependent apoptosis.

17. A pharmaceutical composition comprising the trimeric complex of 13 and at least one pharmaceutically acceptable excipient.

18. A method for treating a cancer patient comprising administering to a patient in need thereof the pharmaceutical composition of claim 17.

19. A DR4 receptor agonist comprising the complex of claim 13.


SEQUENCE LISTING (570 sequences)

SEQ ID NO: 1 - protein, 52 aa, Artificial Sequence
  Synthetic
EPPTQKPKKIVNAKKDVVNTKMFEELKSRLDTLAQEVALLKEQQALQTVCLK

SEQ ID NO: 2 - protein, 58 aa, Artificial Sequence
  Synthetic
NTGLLESQLSRHDQMLSVHDIRLADMDLRFQVLETASYNGVLIWKIRDYKRRKQEAVM

SEQ ID NO: 3 - protein, 21 aa, Artificial Sequence
  Synthetic
AASERKALQTEMARIKKWLTF

SEQ ID NO: 4 - protein, 37 aa, Artificial Sequence
  Synthetic
FDMSCRSRLATLNEKLTALERRIEYIEARVTKGETLT

SEQ ID NO: 5 - protein, 49 aa, Artificial Sequence
  Synthetic
ADIYKADFQAERQAREKLAEKKELLQEQLEQLQREYSKLKASCQESARI

SEQ ID NO: 6 - protein, 71 aa, Artificial Sequence
  Synthetic
LTGSAQNIEFRTGSLGKIKLNDEDLSECLHQIQKNKEDIIELKGSAIGLPIYQLNSKLVDLERKFQGLQQT

SEQ ID NO: 7 - protein, 28 aa, Artificial Sequence
  Synthetic
LRGLRTIVTTLQDSIRKVTEENKELANE

SEQ ID NO: 8 - protein, 27 aa, Artificial Sequence
  Synthetic
VASLRQQVEALQGQVQHLQAAFSQYKK

SEQ ID NO: 9 - protein, 27 aa, Artificial Sequence
  Synthetic
VNALRQRVGILEGQLQRLQNAFSQYKK

SEQ ID NO: 10 - protein, 36 aa, Artificial Sequence
  Synthetic
XXXXXXXXXLXXXXXXLXXEVXXLKEXQALQTVCLX

SEQ ID NO: 11 - protein, 27 aa, Artificial Sequence
  Synthetic
SAALRQQMEALNGKLQRLEAAFSRYKK

SEQ ID NO: 12 - protein, 27 aa, Artificial Sequence
  Synthetic
VNALKQRVTILDGHLRRFQNAFSQYKK

SEQ ID NO: 13 - protein, 27 aa, Artificial Sequence
  Synthetic
VDTLRQRMRNLEGEVQRLQNIVTQYRK

SEQ ID NO: 14 - protein, 64 aa, Artificial Sequence
  Synthetic
GSPGLKGDKGIPGDKGAKGESGLPDVASLRQQVEALQGQVQHLQAAFSQYKKVELFPGGIPHRD

SEQ ID NO: 15 - DNA, 546 bp, Artificial Sequence
  Synthetic
gagtcacccactcccaaggccaagaaggctgcaaatgccaagaaagatttggtgagctcaaagatgttcgaggagctcaagaacaggatggatgtcctggcccaggaggtggccctgctgaaggagaagcaggccttacagactgtgtgcctgaagggcaccaaggtgaacttgaagtgcctcctggccttcacccaaccgaagaccttccatgaggcgagcgaggactgcatctcgcaagggggcacgctgggcaccccgcagtcagagctagagaacgaggcgctgttcgagtacgcgcgccacagcgtgggcaacgatgcgaacatctggctgggcctcaacgacatggccgcggaaggcgcctgggtggacatgaccggcggcctcctggcctacaagaactgggagacggagatcacgacgcaacccgacggcggcaaagccgagaactgcgccgccctgtctggcgcagccaacggcaagtggttcgacaagcgatgccgcgatcagttgccctacatctgccagtttgccattgtgtag

SEQ ID NO: 16 - protein, 181 aa, Artificial Sequence
  Synthetic
ESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 17 - protein, 52 aa, Artificial Sequence
  Synthetic
ESPTPKAKKAANAKKDLVSSKMFEELKNRMDVLAQEVALLKEKQALQTVCLK

SEQ ID NO: 18 - protein, 52 aa, Artificial Sequence
  Synthetic
QQNGKGRQKPAASKKDGVSLKMIEDLKAMIDNISQEVALLKEKQALQTVCLK

SEQ ID NO: 19 - protein, 52 aa, Artificial Sequence
  Synthetic
ETPTPKAKKAANAKKDAVSPKMLEELKTQLDSLAQEVALLKEQQALQTVCLK

SEQ ID NO: 20 - protein, 49 aa, Artificial Sequence
  Synthetic
QQTSSKKKGGKKDAENNAAIEELKKQIDNIVLELNLLKEQQALQSVCLK

SEQ ID NO: 21 - protein, 49 aa, Artificial Sequence
  Synthetic
QQNGKKNKQNNKDVVSMKMYEDLKKKVQNIEEDVIHLKEQQALQTICLK

SEQ ID NO: 22 - protein, 48 aa, Artificial Sequence
  Synthetic
EQSLTKRKNGKKESNSAAIEELKKQIDQIIQDLNLLKEQQALQTVCLK

SEQ ID NO: 23 - protein, 52 aa, Artificial Sequence
  Synthetic
QTSCHASKFKARKHSKRRVKEKDGDLKTQVEKLWREVNALKEMQALQTVCLR

SEQ ID NO: 24 - protein, 38 aa, Artificial Sequence
  Synthetic
KPSKSGKGKDDLRNEIDKLWREVNSLKEMQALQTVCLK

SEQ ID NO: 25 - protein, 54 aa, Artificial Sequence
  Synthetic
EPPTQKPKKIVNAKKDVVNTKMFEELKARLDTLSQEVALLKEQQALQTVSLKGS

SEQ ID NO: 26 - protein, 49 aa, Artificial Sequence
  Synthetic
EPPTQKPKKIVNAKKDVVNTKMFEELKSRLDTLAQEVALLKEQQALQTV

SEQ ID NO: 27 - protein, 47 aa, Artificial Sequence
  Synthetic
KPKKIVNAKKDVVNTKMFEELKSRLDTLSQEVALLKEQQALQTVSLK

SEQ ID NO: 28 - protein, 43 aa, Artificial Sequence
  Synthetic
IVNAKKDVVNTKMFEELKSRLDTLSQEVALLKEQQALQTVSLK

SEQ ID NO: 29 - protein, 37 aa, Artificial Sequence
  Synthetic
DVVNTKMFEELKSRLDTLSQEVALLKEQQALQTVSLK

SEQ ID NO: 30 - protein, 33 aa, Artificial Sequence
  Synthetic
TKMFEELKSRLDTLSQEVALLKEQQALQTVSLK

SEQ ID NO: 31 - protein, 29 aa, Artificial Sequence
  Synthetic
EELKSRLDTLSQEVALLKEQQALQTVSLK

SEQ ID NO: 32 - protein, 25 aa, Artificial Sequence
  Synthetic
SRLDTLSQEVALLKEQQALQTVSLK

SEQ ID NO: 33 - protein, 43 aa, Artificial Sequence
  Synthetic
KPKKIVNAKKDVVNTKMFEELKSRLDTLSQEVALLKEQQALQT

SEQ ID NO: 34 - protein, 41 aa, Artificial Sequence
  Synthetic
KPKKIVNAKKDVVNTKMFEELKSRLDTLSQEVALLKEQQAL

SEQ ID NO: 35 - protein, 38 aa, Artificial Sequence
  Synthetic
KPKKIVNAKKDVVNTKMFEELKSRLDTLSQEVALLKEQ

SEQ ID NO: 36 - protein, 34 aa, Artificial Sequence
  Synthetic
KPKKIVNAKKDVVNTKMFEELKSRLDTLSQEVAL

SEQ ID NO: 37 - protein, 31 aa, Artificial Sequence
  Synthetic
KPKKIVNAKKDVVNTKMFEELKSRLDTLSQE

SEQ ID NO: 38 - protein, 40 aa, Artificial Sequence
  Synthetic
IVNAKKDVVNTKMFEELKSRLDTLAQEVALLKEQQALQTV

SEQ ID NO: 39 - protein, 33 aa, Artificial Sequence
  Synthetic
VVNTKMFEELKSRLDTLAQEVALLKEQQALQTV

SEQ ID NO: 40 - protein, 53 aa, Artificial Sequence
  Synthetic
EPPTQKPKKIVNAKKDVVNTKMFEELKSRLDTLAQEVALLKEQQALQTVSLKG

SEQ ID NO: 41 - protein, 52 aa, Artificial Sequence
  Synthetic
EPPTQKPKKIVNAKKDVVNTKMFEELKSRLDTLAQEVALLKEQQALQTVSLK

SEQ ID NO: 42 - protein, 51 aa, Artificial Sequence
  Synthetic
EPPTQKPKKIVNAKKDVVNTKMFEELKSRLDTLAQEVALLKEQQALQTVSL

SEQ ID NO: 43 - protein, 50 aa, Artificial Sequence
  Synthetic
EPPTQKPKKIVNAKKDVVNTKMFEELKSRLDTLAQEVALLKEQQALQTVS

SEQ ID NO: 44 - protein, 49 aa, Artificial Sequence; identical to SEQ ID NO: 26
  Synthetic

SEQ ID NO: 45 - protein, 48 aa, Artificial Sequence
  Synthetic
EPPTQKPKKIVNAKKDVVNTKMFEELKSRLDTLAQEVALLKEQQALQT

SEQ ID NO: 46 - protein, 52 aa, Artificial Sequence
  Synthetic
PPTQKPKKIVNAKKDVVNTKMFEELKSRLDTLAQEVALLKEQQALQTVSLKG

SEQ ID NO: 47 - protein, 48 aa, Artificial Sequence
  Synthetic
PPTQKPKKIVNAKKDVVNTKMFEELKSRLDTLAQEVALLKEQQALQTV

SEQ ID NO: 48 - protein, 51 aa, Artificial Sequence
  Synthetic
PTQKPKKIVNAKKDVVNTKMFEELKSRLDTLAQEVALLKEQQALQTVSLKG

SEQ ID NO: 49 - protein, 50 aa, Artificial Sequence
  Synthetic
TQKPKKIVNAKKDVVNTKMFEELKSRLDTLAQEVALLKEQQALQTVSLKG

SEQ ID NO: 50 - protein, 49 aa, Artificial Sequence
  Synthetic
QKPKKIVNAKKDVVNTKMFEELKSRLDTLAQEVALLKEQQALQTVSLKG

SEQ ID NO: 51 - protein, 48 aa, Artificial Sequence
  Synthetic
KPKKIVNAKKDVVNTKMFEELKSRLDTLAQEVALLKEQQALQTVSLKG

SEQ ID NO: 52 - protein, 47 aa, Artificial Sequence
  Synthetic
PKKIVNAKKDVVNTKMFEELKSRLDTLAQEVALLKEQQALQTVSLKG

SEQ ID NO: 53 - protein, 46 aa, Artificial Sequence
  Synthetic
KKIVNAKKDVVNTKMFEELKSRLDTLAQEVALLKEQQALQTVSLKG

SEQ ID NO: 54 - protein, 45 aa, Artificial Sequence
  Synthetic
KIVNAKKDVVNTKMFEELKSRLDTLAQEVALLKEQQALQTVSLKG

SEQ ID NO: 55 - protein, 44 aa, Artificial Sequence
  Synthetic
IVNAKKDVVNTKMFEELKSRLDTLAQEVALLKEQQALQTVSLKG

SEQ ID NO: 56 - protein, 43 aa, Artificial Sequence
  Synthetic
VNAKKDVVNTKMFEELKSRLDTLAQEVALLKEQQALQTVSLKG

SEQ ID NO: 57 - protein, 42 aa, Artificial Sequence
  Synthetic
NAKKDVVNTKMFEELKSRLDTLAQEVALLKEQQALQTVSLKG

SEQ ID NO: 58 - protein, 41 aa, Artificial Sequence
  Synthetic
AKKDVVNTKMFEELKSRLDTLAQEVALLKEQQALQTVSLKG

SEQ ID NO: 59 - protein, 40 aa, Artificial Sequence
  Synthetic
KKDVVNTKMFEELKSRLDTLAQEVALLKEQQALQTVSLKG

SEQ ID NO: 60 - protein, 39 aa, Artificial Sequence
  Synthetic
KDVVNTKMFEELKSRLDTLAQEVALLKEQQALQTVSLKG

SEQ ID NO: 61 - protein, 37 aa, Artificial Sequence
  Synthetic
VVNTKMFEELKSRLDTLAQEVALLKEQQALQTVSLKG

SEQ ID NO: 62 - protein, 36 aa, Artificial Sequence
  Synthetic
VNTKMFEELKSRLDTLAQEVALLKEQQALQTVSLKG

SEQ ID NO: 63 - protein, 35 aa, Artificial Sequence
  Synthetic
VNTKMFEELKSRLDTLAQEVALLKEQQALQTVSLK

SEQ ID NO: 64 - protein, 34 aa, Artificial Sequence
  Synthetic
NTKMFEELKSRLDTLAQEVALLKEQQALQTVSLK

SEQ ID NO: 65 - protein, 33 aa, Artificial Sequence
  Synthetic
TKMFEELKSRLDTLAQEVALLKEQQALQTVSLK

SEQ ID NO: 66 - protein, 32 aa, Artificial Sequence
  Synthetic
KMFEELKSRLDTLAQEVALLKEQQALQTVSLK

SEQ ID NO: 67 - protein, 31 aa, Artificial Sequence
  Synthetic
MFEELKSRLDTLAQEVALLKEQQALQTVSLK

SEQ ID NO: 68 - protein, 33 aa, Artificial Sequence; identical to SEQ ID NO: 39
  Synthetic

SEQ ID NO: 69 - protein, 32 aa, Artificial Sequence
  Synthetic
VVNTKMFEELKSRLDTLAQEVALLKEQQALQT

SEQ ID NO: 70 - protein, 30 aa, Artificial Sequence
  Synthetic
VNTKMFEELKSRLDTLAQEVALLKEQQALQ

SEQ ID NO: 71 - protein, 35 aa, Artificial Sequence
  Synthetic
NTKMFEELKSRLDTLAQEVALLKEQQALQTVSLKG

SEQ ID NO: 72 - protein, 34 aa, Artificial Sequence
  Synthetic
TKMFEELKSRLDTLAQEVALLKEQQALQTVSLKG

SEQ ID NO: 73 - protein, 33 aa, Artificial Sequence
  Synthetic
KMFEELKSRLDTLAQEVALLKEQQALQTVSLKG

SEQ ID NO: 74 - protein, 32 aa, Artificial Sequence
  Synthetic
MFEELKSRLDTLAQEVALLKEQQALQTVSLKG

SEQ ID NO: 75 - protein, 52 aa, Artificial Sequence
  Synthetic
EGPTQKPKKIVNAKKDVVNTKMFEELKSRLDTLAQEVALLKEQQALQTVSLK

SEQ ID NO: 76 - protein, 49 aa, Artificial Sequence
  Synthetic
EGPTQKPKKIVNAKKDVVNTKMFEELKSRLDTLAQEVALLKEQQALQTV

SEQ ID NO: 77 - protein, 48 aa, Artificial Sequence
  Synthetic
EGPTQKPKKIVNAKKDVVNTKMFEELKSRLDTLAQEVALLKEQQALQT

SEQ ID NO: 78 - protein, 47 aa, Artificial Sequence
  Synthetic
EGPTQKPKKIVNAKKDVVNTKMFEELKSRLDTLAQEVALLKEQQALQ

SEQ ID NO: 79 - protein, 43 aa, Artificial Sequence
  Synthetic
IVNAKKDVVNTKMFEELKSRLDTLAQEVALLKEQQALQTVSLK

SEQ ID NO: 80 - protein, 40 aa, Artificial Sequence; identical to SEQ ID NO: 38
  Synthetic

SEQ ID NO: 81 - protein, 39 aa, Artificial Sequence
  Synthetic
IVNAKKDVVNTKMFEELKSRLDTLAQEVALLKEQQALQT

SEQ ID NO: 82 - protein, 38 aa, Artificial Sequence
  Synthetic
IVNAKKDVVNTKMFEELKSRLDTLAQEVALLKEQQALQ

SEQ ID NO: 83 - protein, 35 aa, Artificial Sequence; identical to SEQ ID NO: 63
  Synthetic

SEQ ID NO: 84 - protein, 32 aa, Artificial Sequence
  Synthetic
VNTKMFEELKSRLDTLAQEVALLKEQQALQTV

SEQ ID NO: 85 - protein, 31 aa, Artificial Sequence
  Synthetic
VNTKMFEELKSRLDTLAQEVALLKEQQALQT

SEQ ID NO: 86 - protein, 30 aa, Artificial Sequence; identical to SEQ ID NO: 70
  Synthetic

SEQ ID NO: 87 - protein, 40 aa, Artificial Sequence
  Synthetic
MIVNAKKDVVNTKMFEELKSRLDTLAQEVALLKEQQALQT

SEQ ID NO: 88 - protein, 32 aa, Artificial Sequence
  Synthetic
MVNTKMFEELKSRLDTLAQEVALLKEQQALQT

SEQ ID NO: 89 - protein, 53 aa, Artificial Sequence
  Synthetic
EPPTQKPKKIVNAKKDVVNTKMFEELKARLDTLSQEVALLKEQQALQTVSLKG

SEQ ID NO: 90 - protein, 52 aa, Artificial Sequence
  Synthetic
EPPTQKPKKIVNAKKDVVNTKMFEELKARLDTLSQEVALLKEQQALQTVSLK

SEQ ID NO: 91 - protein, 51 aa, Artificial Sequence
  Synthetic
EPPTQKPKKIVNAKKDVVNTKMFEELKARLDTLSQEVALLKEQQALQTVSL

SEQ ID NO: 92 - protein, 50 aa, Artificial Sequence
  Synthetic
EPPTQKPKKIVNAKKDVVNTKMFEELKARLDTLSQEVALLKEQQALQTVS

SEQ ID NO: 93 - protein, 49 aa, Artificial Sequence
  Synthetic
EPPTQKPKKIVNAKKDVVNTKMFEELKARLDTLSQEVALLKEQQALQTV

SEQ ID NO: 94 - protein, 52 aa, Artificial Sequence
  Synthetic
PPTQKPKKIVNAKKDVVNTKMFEELKARLDTLSQEVALLKEQQALQTVSLKG

SEQ ID NO: 95 - protein, 51 aa, Artificial Sequence
  Synthetic
PTQKPKKIVNAKKDVVNTKMFEELKARLDTLSQEVALLKEQQALQTVSLKG

SEQ ID NO: 96 - protein, 50 aa, Artificial Sequence
  Synthetic
TQKPKKIVNAKKDVVNTKMFEELKARLDTLSQEVALLKEQQALQTVSLKG

SEQ ID NO: 97 - protein, 49 aa, Artificial Sequence
  Synthetic
QKPKKIVNAKKDVVNTKMFEELKARLDTLSQEVALLKEQQALQTVSLKG

SEQ ID NO: 98 - protein, 48 aa, Artificial Sequence
  Synthetic
KPKKIVNAKKDVVNTKMFEELKARLDTLSQEVALLKEQQALQTVSLKG

SEQ ID NO: 99 - DNA, 546 bp, Artificial Sequence
  Synthetic
gagccaccaacccagaagcccaagaagattgtaaatgccaagaaagatgttgtgaacacaaagatgtttgaggagctcaagagccgtctggacaccctggcccaggaggtggccctgctgaaggagcagcaggccctgcagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggcgcccgcatcgcctacaagaactgggagactgagatcaccgcgcaacccgatggcggcaagaccgagaactgcgcggtcctgtcaggcgcggccaacggcaagtggttcgacaagcgctgccgcgatcagctgccctacatctgccagttcgggatcgtgtag

SEQ ID NO: 100 - protein, 181 aa, Artificial Sequence
  Synthetic
EPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 101 - protein, 47 aa, Artificial Sequence
  Synthetic
PKKIVNAKKDVVNTKMFEELKARLDTLSQEVALLKEQQALQTVSLKG

SEQ ID NO: 102 - protein, 46 aa, Artificial Sequence
  Synthetic
KKIVNAKKDVVNTKMFEELKARLDTLSQEVALLKEQQALQTVSLKG

SEQ ID NO: 103 - protein, 45 aa, Artificial Sequence
  Synthetic
KIVNAKKDVVNTKMFEELKARLDTLSQEVALLKEQQALQTVSLKG

SEQ ID NO: 104 - protein, 44 aa, Artificial Sequence
  Synthetic
IVNAKKDVVNTKMFEELKARLDTLSQEVALLKEQQALQTVSLKG

SEQ ID NO: 105 - protein, 43 aa, Artificial Sequence
  Synthetic
VNAKKDVVNTKMFEELKARLDTLSQEVALLKEQQALQTVSLKG

SEQ ID NO: 106 - protein, 42 aa, Artificial Sequence
  Synthetic
NAKKDVVNTKMFEELKARLDTLSQEVALLKEQQALQTVSLKG

SEQ ID NO: 107 - protein, 41 aa, Artificial Sequence
  Synthetic
AKKDVVNTKMFEELKARLDTLSQEVALLKEQQALQTVSLKG

SEQ ID NO: 108 - protein, 40 aa, Artificial Sequence
  Synthetic
KKDVVNTKMFEELKARLDTLSQEVALLKEQQALQTVSLKG

SEQ ID NO: 109 - protein, 39 aa, Artificial Sequence
  Synthetic
KDVVNTKMFEELKARLDTLSQEVALLKEQQALQTVSLKG

SEQ ID NO: 110 - protein, 37 aa, Artificial Sequence
  Synthetic
VVNTKMFEELKARLDTLSQEVALLKEQQALQTVSLKG

SEQ ID NO: 111 - protein, 36 aa, Artificial Sequence
  Synthetic
VNTKMFEELKARLDTLSQEVALLKEQQALQTVSLKG

SEQ ID NO: 112 - protein, 35 aa, Artificial Sequence
  Synthetic
VNTKMFEELKARLDTLSQEVALLKEQQALQTVSLK

SEQ ID NO: 113 - protein, 34 aa, Artificial Sequence
  Synthetic
NTKMFEELKARLDTLSQEVALLKEQQALQTVSLK

SEQ ID NO: 114 - protein, 33 aa, Artificial Sequence
  Synthetic
TKMFEELKARLDTLSQEVALLKEQQALQTVSLK

SEQ ID NO: 115 - protein, 31 aa, Artificial Sequence
  Synthetic
MFEELKARLDTLSQEVALLKEQQALQTVSLK

SEQ ID NO: 116 - protein, 71 aa, Artificial Sequence
  Synthetic
MGSHHHHHGSIQGRSPGTEPPTQKPKKIVNAKKDVVNTKMFEELKSRLDTLAQEVALLKEQQALQTVSLKG

SEQ ID NO: 117 - protein, 137 aa, Artificial Sequence
  Synthetic
ALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 118 - protein, 126 aa, Artificial Sequence
  Synthetic
NKLHAGSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCSFPA

SEQ ID NO: 119 - protein, 127 aa, Artificial Sequence
  Synthetic
KKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCAF

SEQ ID NO: 120 - protein, 123 aa, Artificial Sequence
  Synthetic
KVYWFCYGMKCYYFVMDRKTWSGCKQTCQSSSLSLLKIDDEDELKFLQLLVVPSDSCWVGLSYDNKKDWAWIDNRPSKLALNTRKYNIRDRGGCMLLSKTRLDNGNCDQVFICICGKRLDKFP

SEQ ID NO: 121 - protein, 128 aa, Artificial Sequence
  Synthetic
CPVNWVEHERSCYWFSRSGKAWADADNYCRLEDAHLVVVTSWEEQLFVQHHIGPVNTWMGLHDQNGPWKWVDGTDYETGFKNWRPEQPDDWYGHGLGGGEDCAHFTDDGRWNDDVCQRPYRWVCSTEL

SEQ ID NO: 122 - protein, 147 aa, Artificial Sequence
  Synthetic
GIPKCPEDWGASSRTSLCFKLYAKGKHEKKTWFESRDFCRALGGDLASINNKEEQQTIWRLITASGSYHKLFWLGLTYGSPSEGFTWSDGSPVSYENWAYGEPNNYQNVEYCGELKGDPTMSWNDINCEHLNNWICQIQKGQTPKPD

SEQ ID NO: 123 - protein, 129 aa, Artificial Sequence
  Synthetic
DCLSGWSSYEGHCYKAFSKYKTWEDAERVCTEQAKGAHLVSIESSGEADFVAQLVTQNMKRLDFYIWIGLRVQGKVKQCNSEWSDGSSVSYENWIEAESKTCLGLEKETDFRKWVNIYCGQQNPFVCEA

SEQ ID NO: 124 - protein, 122 aa, Artificial Sequence
  Synthetic
DCPSDWSSYEGHCYKPFSEPKNWADAENFCTQQHAGGHLVSFQSSEEADFVVKLAFQTFHSIFWMGLSNVWNQCNWQWSNAAMLRYKAWAEESYCVYFKSTNNKWRSRACRMMAQFVCEFQA

SEQ ID NO: 125 - protein, 135 aa, Artificial Sequence
  Synthetic
ARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFGKWKDVPCEDKFSFVCKFKN

SEQ ID NO: 126 - protein, 123 aa, Artificial Sequence
  Synthetic
DYEILFSDETMNYADAGTYCGSRGMALVSSAMRDSTMVKAILAFTEVKGHDYWVGADNLQDGAYNFNWNDGVSLPTDSDLWSPNEPSNPQSWQLCVQIWSKYNLLDDVGCGGARRVICEKELD

SEQ ID NO: 127 - protein, 202 aa, Homo sapiens
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 128 - protein, 202 aa, Mus musculus
MGFWGTYLLFCLFSFLSQLTAESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 129 - protein, 201 aa, Gallus gallus
MALRGACLLLCLVSLAHISVQQNGKGRQKPAASKKDGVSLKMIEDLKAMIDNISQEVALLKEKQALQTVCLKGTKIHLKCFLAFSESKTYHEASEHCISQGGTLGTPQGGEENDALYDYMRKSIGNEAEIWLGLNDMVAEGKWVDMTGSPIRYKNWETEITTQPDGGKLENCAALSGVAVGKWFDKRCKEQLPYVCQFMIV

SEQ ID NO: 130 - protein, 202 aa, Bos taurus
MELWGPCVLLCLFSLLTQVTAETPTPKAKKAANAKKDAVSPKMLEELKTQLDSLAQEVALLKEQQALQTVCLKGTKVHMKCFLAFVQAKTFHEASEDCISRGGTLGTPQTGSENDALYEYLRQSVGSEAEVWLGFNDMASEGSWVDMTGGHIAYKNWETEITAQPDGGKVENCATLSGAANGKWFDKRCRDKLPYVCQFAIV

SEQ ID NO: 131 - protein, 198 aa, Salmo salar
MRVSGVRLLFCLLLLGQSTFQQTSSKKKGGKKDAENNAAIEELKKQIDNIVLELNLLKEQQALQSVCLKGIKIIGKCFLADTAKKIYHTAYDDCIAKGGTISTPLTGDENDQLVDYVRRSIGPEEHIWLGINDMVTEGEWLDQAGTNLRFKNWETDITNQPDGGRTHNCAILSTTANGKWFDESCRVEKASVCEFNIV

SEQ ID NO: 132 - protein, 198 aa, Silurana tropicalis
MEYRRACILLCLFCFVQVTLQQNGKKNKQNNKDVVSMKMYEDLKKKVQNIEEDVIHLKEQQALQTICLKGMKIYNKCFLAFNELKTYHQASDVCFAQGGTLSTPETGDENDSLYDYVRKSIGSSAEIWIGINDMATEGTWLDLTGSPISFKHWETEITTQPDGGKQENCAALSASAIGRWFDKNCKTELPFVCQFSIV

SEQ ID NO: 133 - protein, 223 aa, Danio rerio
MRDDSDKVPSLLTDYILKGCTYAEEKMDLKAVKFLLCVICLVKSSPEQSLTKRKNGKKESNSAAIEELKKQIDQIIQDLNLLKEQQALQTVCLKGFKIPGKCFLVDTVKKDFHSANDDCIAKGGILSTPMSGHENDQLQEYVQQTVGPETHIWLGVNDMIKEGEWIDLTGSPIRFKNWESEITHQPDGGRTHNCAVLSSTANGKWFDEDCRGEKASVCQFNIV

SEQ ID NO: 134 - protein, 197 aa, Bos taurus
MAKNGLVIYILVITLLLDQTSCHASKFKARKHSKRRVKEKDGDLKTQVEKLWREVNALKEMQALQTVCLRGTKFHKKCYLAAEGLKHFHEANEDCISKGGTLVVPRSADEINALRDYGKRSLPGVNDFWLGINDMVAEGKFVDINGLAISFLNWDQAQPNGGKRENCALFSQSAQGKWSDEACHSSKRYICEFTIPQ

SEQ ID NO: 135 - protein, 166 aa, Carcharhinus springeri
SKPSKSGKGKDDLRNEIDKLWREVNSLKEMQALQTVCLKGTKIHKKCYLASRGSKSYHAANEDCIAQGGTLSIPRSSDEGNSLRSYAKKSLVGARDFWIGVNDMTTEGKFVDVNGLPITYFNWDRSKPVGGTRENCVAASTSGQGKWSDDVCRSEKRYICEYLIPV

SEQ ID NO: 136 - protein, 281 aa, Homo sapiens [sequence omitted]

SEQ ID NO: 137 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAKEKIQDLLVDSGKFIYLEDGTGSAVSLE

SEQ ID NO: 138 - protein, 440 aa, Homo sapiens [sequence omitted]

SEQ ID NO: 139 - protein, 259 aa, Homo sapiens
MARIPKTLKFVVVIVAVLLPVLAYSATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMTTSPGTPASSHYLSCTIVGIIVLIVLLIVFV

SEQ ID NO: 140 - protein, 386 aa, Homo sapiens
MGLWGQSVPTASSARAGRYPGARTASGTRPWLLDPKILKFVVFIVAVLLPVRVDSATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHYLIIIVVLVIILAVVVVGFSCRKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 141 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 142 - protein, 31 aa, Artificial Sequence
  Synthetic
KMFEELKSQLDSLAQEVALLKEQQALQTVCL

SEQ ID NO: 143 - protein, 31 aa, Artificial Sequence
  Synthetic
KMFEELKSQVDSLAQEVALLKEQQALQTVCL

SEQ ID NO: 144 - protein, 32 aa, Artificial Sequence
  Synthetic
SKMFEELKNRMDVLAQEVALLKEKQALQTVCL

SEQ ID NO: 145 - protein, 31 aa, Artificial Sequence
  Synthetic
KMFEELKNRLDVLAQEVALLKEKQALQTVCL

SEQ ID NO: 146 - protein, 31 aa, Artificial Sequence
  Synthetic
KMLEELKTQLDSLAQEVALLKEQQALQTVCL

SEQ ID NO: 147 - protein, 27 aa, Artificial Sequence
  Synthetic
DLKTQVEKLWREVNALKEMQALQTVCL

SEQ ID NO: 148 - protein, 27 aa, Artificial Sequence
  Synthetic
DLKTQVEKLWREVNALKEMQALQTVCL

SEQ ID NO: 149 - protein, 27 aa, Artificial Sequence
  Synthetic
DLKTQVEKLWREVNALKEMQALQTVCL

SEQ ID NO: 150 - protein, 27 aa, Artificial Sequence
  Synthetic
DLKTQIEKLWTEVNALKEIQALQTVCL

SEQ ID NO: 151 - protein, 28 aa, Artificial Sequence
  Synthetic
DDLKTQIDKLWREVNALKEIQALQTVCL

SEQ ID NO: 152 - protein, 27 aa, Artificial Sequence
  Synthetic
DLKTQVEKLWREVNALKEMQALQTVCL

SEQ ID NO: 153 - protein, 27 aa, Artificial Sequence
  Synthetic
DLKSQVEKLWREVNALKEMQALQTVCL

SEQ ID NO: 154 - protein, 27 aa, Artificial Sequence
  Synthetic
DLKTQVEKLWREVNALKEMQALQTVCL

SEQ ID NO: 155 - protein, 28 aa, Artificial Sequence
  Synthetic
DDLRNEIDKLWREVNSLKEMQALQTVCL

SEQ ID NO: 156 - protein, 31 aa, Artificial Sequence
  Synthetic
KMIEDLKAMIDNISQEVALLKEKQALQTVCL

SEQ ID NO: 157 - protein, 31 aa, Artificial Sequence; identical to SEQ ID NO: 156
  Synthetic

SEQ ID NO: 158 - protein, 28 aa, Artificial Sequence
  Synthetic
DDMKTQIDKLWQEVNSLKEMQALQTVCL

SEQ ID NO: 159 - protein, 28 aa, Artificial Sequence
  Synthetic
DDLKTQIDKLWREVNALKEMQALQSVCL

SEQ ID NO: 160 - protein, 28 aa, Artificial Sequence
  Synthetic
DDLKSQVEKLWREVNALKEMQALQTVCL

SEQ ID NO: 161 - protein, 28 aa, Artificial Sequence
  Synthetic
DDLKTQIDKLWREVNALKEMQALQSVCL

SEQ ID NO: 162 - protein, 28 aa, Artificial Sequence
  Synthetic
DDVRSQIEKLWQEVNSLKEMQALQTVCL

SEQ ID NO: 163 - protein, 27 aa, Artificial Sequence
  Synthetic
DLKTQIDKLWREINSLKEMQALQTVCL

SEQ ID NO: 164 - protein, 28 aa, Artificial Sequence
  Synthetic
EELRRQVSDLAQELNILKEQQALHTVCL

SEQ ID NO: 165 - protein, 31 aa, Artificial Sequence
  Synthetic
KMYEELKQKVQNIELEVIHLKEQQALQTICL

SEQ ID NO: 166 - protein, 31 aa, Artificial Sequence
  Synthetic
KMYEDLKKKVQNIEEDVIHLKEQQALQTICL

SEQ ID NO: 167 - protein, 28 aa, Artificial Sequence
  Synthetic
EELKKQIDNIVLELNLLKEQQALQSVCL

SEQ ID NO: 168 - protein, 28 aa, Artificial Sequence
  Synthetic
EELKKQIDQIIQDLNLLKEQQALQTVCL

SEQ ID NO: 169 - protein, 28 aa, Artificial Sequence
  Synthetic
EQMQKQINDIVQELNLLKEQQALQAVCL

SEQ ID NO: 170 - protein, 28 aa, Artificial Sequence
  Synthetic
EQMQKQINDIVQELNLLKEQQALQAVCL

SEQ ID NO: 171 - DNA, 27 bp, Artificial Sequence
  Synthetic
gccctccagacggtctgcctgaagggg

SEQ ID NO: 172 - DNA, 27 bp, Artificial Sequence
  Synthetic
gttgaggcccagccagatctcggcctc

SEQ ID NO: 173 - DNA, 75 bp, Artificial Sequence
  Synthetic
gaggccgagatctggctgggcctcaacnnknnknnknnknnknnknnktgggtggacatgaccggcgcgcgcatc

SEQ ID NO: 174 - DNA, 27 bp, Artificial Sequence
  Synthetic
cacgatcccgaactggcagatgtaggg

SEQ ID NO: 175 - protein, 17 aa, Artificial Sequence
  Synthetic
NWEXXXXXXXXGGXXXN

SEQ ID NO: 176 - protein, 5 aa, Artificial Sequence
  Synthetic
AAEGT

SEQ ID NO: 177 - DNA, 21 bp, Artificial Sequence
  Synthetic
nnknnknnknnknnknnknnk

SEQ ID NO: 178 - protein, 8 aa, Artificial Sequence
  Synthetic
DMAAEGTW

SEQ ID NO: 179 - protein, 6 aa, Artificial Sequence
  Synthetic
DMTGAR

SEQ ID NO: 180 - protein, 10 aa, Artificial Sequence
  Synthetic
NWETEITAQP

SEQ ID NO: 181 - protein, 7 aa, Artificial Sequence
  Synthetic
DGGKTEN

SEQ ID NO: 182 - protein, 10 aa, Artificial Sequence
  Synthetic
DMXXXXXXXW

SEQ ID NO: 183 - protein, 6 aa, Artificial Sequence
  Synthetic
DMTGAX

SEQ ID NO: 184 - protein, 7 aa, Artificial Sequence
  Synthetic
DGGATEN

SEQ ID NO: 185 - protein, 8 aa, Artificial Sequence
  Synthetic
DMXXXXXW

SEQ ID NO: 186 - protein, 6 aa, Artificial Sequence
  Synthetic
DMXXXX

SEQ ID NO: 187 - protein, 8 aa, Artificial Sequence
  Synthetic
XXXXXXXW

SEQ ID NO: 188 - protein, 10 aa, Artificial Sequence
  Synthetic
DGGXXXXXEN

SEQ ID NO: 189 - protein, 10 aa, Artificial Sequence
  Synthetic
NWXXXXXXQP

SEQ ID NO: 190 - protein, 11 aa, Artificial Sequence
  Synthetic
NWXXXXXXXQP

SEQ ID NO: 191 - protein, 12 aa, Artificial Sequence
  Synthetic
NWXXXXXXXXQP

SEQ ID NO: 192 - protein, 13 aa, Artificial Sequence
  Synthetic
NWETXXXXXXAQP

SEQ ID NO: 193 - protein, 10 aa, Artificial Sequence
  Synthetic
DGGXXXXXXN

SEQ ID NO: 194 - protein, 10 aa, Artificial Sequence
  Synthetic
NWEXXXXXXX

SEQ ID NO: 195 - protein, 7 aa, Artificial Sequence
  Synthetic
XGGXXXN

SEQ ID NO: 196 - protein, 10 aa, Artificial Sequence
  Synthetic
NWEXXXXXQP

SEQ ID NO: 197 - protein, 11 aa, Artificial Sequence
  Synthetic
DGGXXXXXXXN

SEQ ID NO: 198 - DNA, 62 bp, Artificial Sequence
  Synthetic
ggctgggcctgaacgacatgnnknnknnknnknnknnknnktgggtggatatgactggcgcc

SEQ ID NO: 199 - DNA, 60 bp, Artificial Sequence
  Synthetic
ggcggtgatctcagtttcccagttcttgtaggcgatmnnggcgccagtcatatccaccca

SEQ ID NO: 200 - DNA, 62 bp, Artificial Sequence
  Synthetic
actgggaaactgagatcaccgcccaacctgatggcggcgcaaccgagaactgcgcggtcctg

SEQ ID NO: 201 - DNA, 60 bp, Artificial Sequence
  Synthetic
ccctgcagcgcttgtcgaaccacttgccgttggcggcgccagacaggaccgcgcagttct

SEQ ID NO: 202 - DNA, 30 bp, Artificial Sequence
  Synthetic
gccgagatctggctgggcctgaacgacatg

SEQ ID NO: 203 - DNA, 23 bp, Artificial Sequence
  Synthetic
atccctgcagcgcttgtcgaacc

SEQ ID NO: 204 - DNA, 67 bp, Artificial Sequence
  Synthetic
gctgttcgaatacgcgcgccacagcgtgggcaacgatgcgaacatctggctgggcctcaacgatatg

SEQ ID NO: 205 - DNA, 64 bp, Artificial Sequence
  Synthetic
gccgccggtcatgtcgacccamnnmnnmnnmnnmnnmnnmnncatatcgttgaggcccagccag

SEQ ID NO: 206 - DNA, 89 bp, Artificial Sequence
  Synthetic
tgggtcgacatgaccggcggcnnkctggcctacaagaactgggagacggagatcacgacgcaacccgacggcggcgctgccgagaactg

SEQ ID NO: 207 - DNA, 76 bp, Artificial Sequence
  Synthetic
cagcgtttgtcgaaccacttgccgttggctgcgccagacagggcggcgcagttctcggcagcgccgccgtcgggtt

SEQ ID NO: 208 - DNA, 29 bp, Artificial Sequence
  Synthetic
gctgttcgaatacgcgcgccacagcgtgg

SEQ ID NO: 209 - DNA, 41 bp, Artificial Sequence
  Synthetic
gggcaactgatctctgcagcgtttgtcgaaccacttgccgt

SEQ ID NO: 210 - DNA, 79 bp, Artificial Sequence
  Synthetic
ggctgggcctgaacgacatgnnknnknnknnknnktgggtggatatgnnknnknnknnkatcgcctacaagaactggga

SEQ ID NO: 211 - DNA, 77 bp, Artificial Sequence
  Synthetic
gacaggacggcgcagttctcggttgcgccgccatcaggttgggcggtgatctcagtttcccagttcttgtaggcgat

SEQ ID NO: 212 - DNA, 63 bp, Artificial Sequence
  Synthetic
atccctgcagcgcttgtcgaaccacttgccgttggcggcgccagacaggacggcgcagttctc

SEQ ID NO: 213 - DNA, 85 bp, Artificial Sequence
  Synthetic
cgtctcccagttcttgtaggccagmnnmnnmnnmnncatgtcgacccamnnmnnmnnmnnmnncatatcgttgaggcccagccag

SEQ ID NO: 214 - DNA, 62 bp, Artificial Sequence
  Synthetic
gcctacaagaactgggagacggagatcacgacgcaacccgacggcggcgctgccgagaactg

SEQ ID NO: 215 - DNA, 60 bp, Artificial Sequence
  Synthetic
gagatctggctgggcctcaacnnsnnsnnsnnsnnsnnsnnstgggtggacatgactggc

SEQ ID NO: 216 - DNA, 63 bp, Artificial Sequence
  Synthetic
ttgcgcggtgatctcagtctcccagttcttgtaggcgatacgcgcgccagtcatgtccaccca

SEQ ID NO: 217 - DNA, 64 bp, Artificial Sequence
  Synthetic
gactgagatcaccgcgcaacccgatggcggcnnsnnsnnsnnsnnsgagaactgcgcggtcctg

SEQ ID NO: 218 - DNA, 60 bp, Artificial Sequence
  Synthetic
ccctgcagcgcttgtcgaaccacttgccgttggccgcgcctgacaggaccgcgcagttct

SEQ ID NO: 219 - DNA, 22 bp, Artificial Sequence
  Synthetic
gccgagatctggctgggcctca

SEQ ID NO: 220 - DNA, 33 bp, Artificial Sequence
  Synthetic
gccatggccgccttacagactgtgtgcctgaag

SEQ ID NO: 221 - DNA, 87 bp, Artificial Sequence
  Synthetic
cgtctcccagttcttgtaggccaggaggccgccggtcatgtccacccamnnmnnmnnmnnmnnmnnmnngttgaggcccagccagat

SEQ ID NO: 222 - DNA, 81 bp, Artificial Sequence
  Synthetic
gcctacaagaactgggagacggagatcacgacgcaacccgacggcggcnnknnknnknnknnkgagaactgcgccgccctg

SEQ ID NO: 223 - DNA, 38 bp, Artificial Sequence
  Synthetic
cgcacctgcggccgccacaatggcaaactggcagatgt

SEQ ID NO: 224 - DNA, 78 bp, Artificial Sequence
  Synthetic
atctggctgggcctgaacgacatggccgccgagggcacctgggtggatatgaccggcgcgcgtatcgcctacaagaac

SEQ ID NO: 225 - DNA, 62 bp, Artificial Sequence
  Synthetic
ccgccatcgggttgggcmnnmnnmnnmnnmnnmnnagtttcccagttcttgtaggcgatacg

SEQ ID NO: 226 - DNA, 57 bp, Artificial Sequence
  Synthetic
gcccaacccgatggcggcnnknnknnknnknnknnkaactgcgccgtcctgtctggc

SEQ ID NO: 227 - DNA, 54 bp, Artificial Sequence
  Synthetic
cctgcagcgcttgtcgaaccacttgccgttggcggcgccagacaggacggcgca

SEQ ID NO: 228 - DNA, 60 bp, Artificial Sequence
  Synthetic
gacatggccgcggaaggcgcctgggtcgacatgaccggcggcctgctggcctacaagaac

SEQ ID NO: 229 - DNA, 61 bp, Artificial Sequence
  Synthetic
ccgccgtcgggttgggtmnnmnnmnnmnnmnnmnnggtctcccagttcttgtaggccagca

SEQ ID NO: 230 - DNA, 57 bp, Artificial Sequence
  Synthetic
acccaacccgacggcggcnnknnknnknnknnknnkaactgcgccgccctgtctggc

SEQ ID NO: 231 - DNA, 63 bp, Artificial Sequence
  Synthetic
ctgatctctgcagcgcttgtcgaaccacttgccgttggctgcgccagacagggcggcgcagtt

SEQ ID NO: 232 - DNA, 84 bp, Artificial Sequence
  Synthetic
gccagacaggacggcgcagttmnnmnnmnngccgccmnnmnnmnnmnnmnnmnnmnnmnnttcccagttcttgtaggcgatacg

SEQ ID NO: 233 - DNA, 83 bp, Artificial Sequence
  Synthetic
gccagacagggcggcgcagttmnnmnnmnngccgccmnnmnnmnnmnnmnnmnnmnnmnnctcccagttcttgtaggccagca

SEQ ID NO: 234 - DNA, 53 bp, Artificial Sequence
  Synthetic
ccgccatcgggttgggcggtgatctcagtttcccagttcttgtaggcgatacg

SEQ ID NO: 235 - DNA, 60 bp, Artificial Sequence
  Synthetic
gcccaacccgatggcggcnnknnknnknnknnknnknnkaactgcgccgtcctgtctggc

SEQ ID NO: 236 - DNA, 52 bp, Artificial Sequence
  Synthetic
ccgccgtcgggttgggtggtgatctcggtctcccagttcttgtaggccagca

SEQ ID NO: 237 - DNA, 60 bp, Artificial Sequence
  Synthetic
acccaacccgacggcggcnnknnknnknnknnknnknnkaactgcgccgccctgtctggc

SEQ ID NO: 238 - DNA, 74 bp, Artificial Sequence
  Synthetic
ctggcgcgcgtatcgcctacaagaactggnnknnknnknnknnknnkcaacccgatggcggcgccaccgagaac

SEQ ID NO: 239 - DNA, 77 bp, Artificial Sequence
  Synthetic
ctggcgcgcgtatcgcctacaagaactggnnknnknnknnknnknnknnkcaacccgatggcggcgccaccgagaac

SEQ ID NO: 240 - DNA, 77 bp, Artificial Sequence; identical to SEQ ID NO: 239
  Synthetic

SEQ ID NO: 241 - DNA, 81 bp, Artificial Sequence
  Synthetic
cctgcagcgcttgtcgaaccacttgccgttggcggcgccagacaggacggcgcagttctcggtggcgccgccatcgggttg

SEQ ID NO: 242 - DNA, 77 bp, Artificial Sequence
  Synthetic
gttctcggcagcgccgccgtcgggttgmnnmnnmnnmnnmnnmnnccagttcttgtaggccagcaggccgccggtca

SEQ ID NO: 243 - DNA, 80 bp, Artificial Sequence
  Synthetic
gttctcggcagcgccgccgtcgggttgmnnmnnmnnmnnmnnmnnmnnccagttcttgtaggccagcaggccgccggtca

SEQ ID NO: 244 - DNA, 83 bp, Artificial Sequence
  Synthetic
gttctcggcagcgccgccgtcgggttgmnnmnnmnnmnnmnnmnnmnnmnnccagttcttgtaggccagcaggccgccggtca

SEQ ID NO: 245 - DNA, 89 bp, Artificial Sequence
  Synthetic
gacaggaccgcgcagttctcgccsmagwmcccsaagccgccmnngggttgmnnmnnmnnmnnmnnctcccagttcttgtaggcgatacg

SEQ ID NO: 246 - DNA, 66 bp, Artificial Sequence
  Synthetic
atccctgcagcgcttgtcgaaccacttgccgttggccgcgcctgacaggaccgcgcagttctcgcc

SEQ ID NO: 247 - DNA, 15 bp, Artificial Sequence
  Synthetic
nnknnknnknnknnk

SEQ ID NO: 248 - DNA, 12 bp, Artificial Sequence
  Synthetic
nnknnknnknnk

SEQ ID NO: 249 - protein, 7 aa, Artificial Sequence
  Synthetic
DMAAEGT

SEQ ID NO: 250 - DNA, 21 bp, Artificial Sequence
  Synthetic
nnsnnsnnsnnsnnsnnsnns

SEQ ID NO: 251 - DNA, 15 bp, Artificial Sequence
  Synthetic
nnsnnsnnsnnsnns

SEQ ID NO: 252 - DNA, 18 bp, Artificial Sequence
  Synthetic
nnknnknnknnknnknnk

SEQ ID NO: 253 - protein, 13 aa, Artificial Sequence
  Synthetic
TEITAQPDGGKTE

SEQ ID NO: 254 - protein, 13 aa, Artificial Sequence
  Synthetic
XXXXXXXXGGXXX

SEQ ID NO: 255 - protein, 6 aa, Artificial Sequence
  Synthetic
ETEITA

SEQ ID NO: 256 - DNA, 24 bp, Artificial Sequence
  Synthetic
nnknnknnknnknnknnknnknnk

SEQ ID NO: 257 - DNA, 22 bp, Artificial Sequence
  Synthetic
gccgagatctggctgggcctga

SEQ ID NO: 258 - protein, 5 aa, Artificial Sequence
  Synthetic
TEITA

SEQ ID NO: 259 - protein, 8 aa, Artificial Sequence
  Synthetic
GWLEGAGW

SEQ ID NO: 260 - protein, 10 aa, Artificial Sequence
  Synthetic
DGGWHWRWEN

SEQ ID NO: 261 - protein, 8 aa, Artificial Sequence
  Synthetic
GWLEGVGW

SEQ ID NO: 262 - protein, 10 aa, Artificial Sequence
  Synthetic
DGGEHWGWEN

SEQ ID NO: 263 - protein, 8 aa, Artificial Sequence
  Synthetic
GYLAGVGW

SEQ ID NO: 264 - protein, 10 aa, Artificial Sequence
  Synthetic
DGGRGFRWEN

SEQ ID NO: 265 - protein, 8 aa, Artificial Sequence
  Synthetic
GWLEGYGW

SEQ ID NO: 266 - protein, 10 aa, Artificial Sequence
  Synthetic
DGGTWWEWEN

SEQ ID NO: 267 - protein, 8 aa, Artificial Sequence
  Synthetic
GYLEGYGW

SEQ ID NO: 268 - protein, 10 aa, Artificial Sequence
  Synthetic
DGGATIAWEN

SEQ ID NO: 269 - protein, 8 aa, Artificial Sequence
  Synthetic
GWLQGVGW

SEQ ID NO: 270 - protein, 10 aa, Artificial Sequence
  Synthetic
DGGRGWPWEN

SEQ ID NO: 271 - protein, 8 aa, Artificial Sequence
  Synthetic
GYLAGYGW

SEQ ID NO: 272 - protein, 10 aa, Artificial Sequence
  Synthetic
DGGPSIWREN

SEQ ID NO: 273 - protein, 8 aa, Artificial Sequence
  Synthetic
GYIEGTGW

SEQ ID NO: 274 - protein, 10 aa, Artificial Sequence
  Synthetic
DGGSNWAWEN

SEQ ID NO: 275 - protein, 8 aa, Artificial Sequence
  Synthetic
GYMSGYGW

SEQ ID NO: 276 - protein, 10 aa, Artificial Sequence
  Synthetic
DGGMMARWEN

SEQ ID NO: 277 - protein, 8 aa, Artificial Sequence
  Synthetic
GFMVGRGW

SEQ ID NO: 278 - protein, 10 aa, Artificial Sequence
  Synthetic
DGGSMWPWEN

SEQ ID NO: 279 - protein, 8 aa, Artificial Sequence
  Synthetic
MVTRPPYW

SEQ ID NO: 280 - protein, 10 aa, Artificial Sequence
  Synthetic
DGGWVMSFEN

SEQ ID NO: 281 - protein, 8 aa, Artificial Sequence
  Synthetic
PFRVPQWW

SEQ ID NO: 282 - protein, 10 aa, Artificial Sequence
  Synthetic
DGGYGPVQEN

SEQ ID NO: 283 - protein, 10 aa, Artificial Sequence
  Synthetic
DGGWQWRWEN

SEQ ID NO: 284 - protein, 8 aa, Artificial Sequence
  Synthetic
GYLDGVGW

SEQ ID NO: 285 - protein, 10 aa, Artificial Sequence
  Synthetic
DGGQGCRWEN

SEQ ID NO: 286 - protein, 8 aa, Artificial Sequence
  Synthetic
VLRLAWSW

SEQ ID NO: 287 - protein, 10 aa, Artificial Sequence
  Synthetic
DGGKRNGCEN

SEQ ID NO: 288 - protein, 8 aa, Artificial Sequence
  Synthetic
WLSLFSPW

SEQ ID NO: 289 - protein, 10 aa, Artificial Sequence
  Synthetic
DGGRGVRGEN

SEQ ID NO: 290 - protein, 8 aa, Artificial Sequence
  Synthetic
GWMAGVGW

SEQ ID NO: 291 - protein, 10 aa, Artificial Sequence
  Synthetic
DGGRRLPWEN

SEQ ID NO: 292 - protein, 8 aa, Artificial Sequence
  Synthetic
SYRLHYGW

SEQ ID NO: 293 - protein, 10 aa, Artificial Sequence
  Synthetic
DGGRRWLGEN

SEQ ID NO: 294 - protein, 8 aa, Artificial Sequence
  Synthetic
IWPLRFRW

SEQ ID NO: 295 - protein, 10 aa, Artificial Sequence
  Synthetic
DGGFVTRKEN

SEQ ID NO: 296 - protein, 8 aa, Artificial Sequence
  Synthetic
WQLYYRYW

SEQ ID NO: 297 - protein, 10 aa, Artificial Sequence
  Synthetic
DGGVGCMVEN

SEQ ID NO: 298 - protein, 8 aa, Artificial Sequence
  Synthetic
RCLQGVGW

SEQ ID NO: 299 - protein, 8 aa, Artificial Sequence
  Synthetic
GCTQGQGW

SEQ ID NO: 300 - protein, 10 aa, Artificial Sequence
  Synthetic
DGGKKWKWEN

SEQ ID NO: 301 - protein, 8 aa, Artificial Sequence
  Synthetic
GFLQGNGW

SEQ ID NO: 302 - protein, 10 aa, Artificial Sequence
  Synthetic
DGGMWDRWEN

SEQ ID NO: 303 - protein, 8 aa, Artificial Sequence
  Synthetic
GVLQRGGW

SEQ ID NO: 304 - protein, 10 aa, Artificial Sequence
  Synthetic
DGGPGGEREN

SEQ ID NO: 305 - protein, 9 aa, Artificial Sequence
  Synthetic
PFRVLQQWW

SEQ ID NO: 306 - protein, 11 aa, Artificial Sequence
  Synthetic
DGGCGPVQQEN

SEQ ID NO: 307 - protein, 9 aa, Artificial Sequence
  Synthetic
PFRGPQQWW

SEQ ID NO: 308 - protein, 10 aa, Artificial Sequence
  Synthetic
DGGYGPVGEN

SEQ ID NO: 309 - protein, 9 aa, Artificial Sequence
  Synthetic
ARFAMWQQW

SEQ ID NO: 310 - protein, 10 aa, Artificial Sequence
  Synthetic
DGGRAGVGEN

SEQ ID NO: 311 - protein, 8 aa, Artificial Sequence
  Synthetic
GWLQGYGW

SEQ ID NO: 312 - protein, 11 aa, Artificial Sequence
  Synthetic
DGGQQIGWGEN

SEQ ID NO: 313 - protein, 8 aa, Artificial Sequence
  Synthetic
AWRSWLNW

SEQ ID NO: 314 - protein, 11 aa, Artificial Sequence
  Synthetic
DGGREQQRREN

SEQ ID NO: 315 - protein, 10 aa, Artificial Sequence
  Synthetic
DGGWPFSNEN

SEQ ID NO: 316 - protein, 8 aa, Artificial Sequence
  Synthetic
GWLMGTGW

SEQ ID NO: 317 - protein, 10 aa, Artificial Sequence
  Synthetic
DGGWWNRWEN

SEQ ID NO: 318 - protein, 8 aa, Artificial Sequence
  Synthetic
VRRMGFHW

SEQ ID NO: 319 - protein, 10 aa, Artificial Sequence
  Synthetic
DGGRVAVGEN

SEQ ID NO: 320 - protein, 8 aa, Artificial Sequence
  Synthetic
RYHVQALW

SEQ ID NO: 321 - protein, 10 aa, Artificial Sequence
  Synthetic
DGGRVRPREN

SEQ ID NO: 322 - protein, 8 aa, Artificial Sequence
  Synthetic
IQCSPPLW

SEQ ID NO: 323 - protein, 10 aa, Artificial Sequence
  Synthetic
DGGAVQQQEN

SEQ ID NO: 324 - protein, 8 aa, Artificial Sequence
  Synthetic
GLARQQGW

SEQ ID NO: 325 - protein, 10 aa, Artificial Sequence
  Synthetic
DGGKGRPREN

SEQ ID NO: 326 - protein, 8 aa, Artificial Sequence
  Synthetic
GWLSGVGW

SEQ ID NO: 327 - protein, 10 aa, Artificial Sequence
  Synthetic
DGGWAHAWEN

SEQ ID NO: 328 - protein, 10 aa, Artificial Sequence
  Synthetic
DGGGGVRWEN

SEQ ID NO: 329 - protein, 8 aa, Artificial Sequence
  Synthetic
GWLSGYGW

SEQ ID NO: 330 - protein, 10 aa, Artificial Sequence
  Synthetic
DGGRVWSWEN

SEQ ID NO: 331 - protein, 8 aa, Artificial Sequence
  Synthetic
GLLSDWWW

SEQ ID NO: 332 - protein, 10 aa, Artificial Sequence
  Synthetic
DGGGNQSREN

SEQ ID NO: 333 - protein, 8 aa, Artificial Sequence
  Synthetic
QWVAFWSW

SEQ ID NO: 334 - protein, 10 aa, Artificial Sequence
  Synthetic
DGGSAVSGEN

SEQ ID NO: 335 - protein, 8 aa, Artificial Sequence
  Synthetic
PYTSWGLW

SEQ ID NO: 336 - protein, 10 aa, Artificial Sequence
  Synthetic
DGGVGGRGEN

SEQ ID NO: 337 - protein, 8 aa, Artificial Sequence
  Synthetic
VARWLLKW

SEQ ID NO: 338 - protein, 10 aa, Artificial Sequence
  Synthetic
DGGMCKPCEN

SEQ ID NO: 339 - protein, 8 aa, Artificial Sequence
  Synthetic
GFLAGVGW

SEQ ID NO: 340 - protein, 10 aa, Artificial Sequence
  Synthetic
DGGWWTRWEN

SEQ ID NO: 341 - protein, 8 aa, Artificial Sequence
  Synthetic
GYLQGSGW

SEQ ID NO: 342 - protein, 10 aa, Artificial Sequence
  Synthetic
DGGWKTRWEN

SEQ ID NO: 343 - protein, 8 aa, Artificial Sequence
  Synthetic
VRHWLQLW

SEQ ID NO: 344 - protein, 10 aa, Artificial Sequence
  Synthetic
DGGGWWKGEN

SEQ ID NO: 345 - protein, 8 aa, Artificial Sequence
  Synthetic
RATLRPRW

SEQ ID NO: 346 - protein, 5 aa, Artificial Sequence
  Synthetic
DGGKN

SEQ ID NO: 347 - protein, 8 aa, Artificial Sequence
  Synthetic
RAMLRSRW

SEQ ID NO: 348 - protein, 10 aa, Artificial Sequence
  Synthetic
DGGRWFQGKN

SEQ ID NO: 349 - protein, 8 aa, Artificial Sequence
  Synthetic
RALFRPRW

SEQ ID NO: 350 - protein, 10 aa, Artificial Sequence
  Synthetic
DGGPWYLKEN

SEQ ID NO: 351 - protein, 8 aa, Artificial Sequence
  Synthetic
RAVLRPRW

SEQ ID NO: 352 - protein, 10 aa, Artificial Sequence
  Synthetic
DGGWVLGGKN

SEQ ID NO: 353 - protein, 8 aa, Artificial Sequence
  Synthetic
RAWLRPRW

SEQ ID NO: 354 - protein, 10 aa, Artificial Sequence
  Synthetic
DGGTLVSGEN

SEQ ID NO: 355 - protein, 8 aa, Artificial Sequence
  Synthetic
RVIRRSMW

SEQ ID NO: 356 - protein, 10 aa, Artificial Sequence
  Synthetic
DGGQKWMAEN

SEQ ID NO: 357 - protein, 8 aa, Artificial Sequence
  Synthetic
RVLQRPVW

SEQ ID NO: 358 - protein, 10 aa, Artificial Sequence
  Synthetic
DGGMVWSMEN

SEQ ID NO: 359 - protein, 8 aa, Artificial Sequence
  Synthetic
RVQLRPRW

SEQ ID NO: 360 - protein, 10 aa, Artificial Sequence
  Synthetic
EGGFRRHAKN

SEQ ID NO: 361 - protein, 8 aa, Artificial Sequence
  Synthetic
RVVRLSEW

SEQ ID NO: 362 - protein, 10 aa, Artificial Sequence
  Synthetic
DGGMLWAMEN

SEQ ID NO: 363 - protein, 8 aa, Artificial Sequence
  Synthetic
RVISAPVW

SEQ ID NO: 364 - protein, 10 aa, Artificial Sequence
  Synthetic
DGGQQWAMEN

SEQ ID NO: 365 - protein, 8 aa, Artificial Sequence
  Synthetic
RVLRRPQW

SEQ ID NO: 366 - protein, 10 aa, Artificial Sequence
  Synthetic
NGGDWRIPEN

SEQ ID NO: 367 - protein, 8 aa, Artificial Sequence
  Synthetic
RVMMRPRW

SEQ ID NO: 368 - protein, 10 aa, Artificial Sequence
  Synthetic
DGGMWGAMEN

SEQ ID NO: 369 - protein, 8 aa, Artificial Sequence
  Synthetic
RVMRRVLW

SEQ ID NO: 370 - protein, 10 aa, Artificial Sequence
  Synthetic
DGGRRETMKN

SEQ ID NO: 371 - protein, 8 aa, Artificial Sequence
  Synthetic
RVMRRPLW

SEQ ID NO: 372 - protein, 10 aa, Artificial Sequence
  Synthetic
DGGRGQQWEN

SEQ ID NO: 373 - protein, 8 aa, Artificial Sequence
  Synthetic
RVMRRREW

SEQ ID NO: 374 - protein, 10 aa, Artificial Sequence
  Synthetic
DGAQLMALEN

SEQ ID NO: 375 - protein, 8 aa, Artificial Sequence
  Synthetic
RVWRRSLW

SEQ ID NO: 376 - protein, 10 aa, Artificial Sequence
  Synthetic
DGGHLVKQKN

SEQ ID NO: 377 - protein, 8 aa, Artificial Sequence
  Synthetic
KRRWYGGW

SEQ ID NO: 378 - protein, 10 aa, Artificial Sequence
  Synthetic
DGGVNTVREN

SEQ ID NO: 379 - protein, 8 aa, Artificial Sequence
  Synthetic
KRVWYRGW

SEQ ID NO: 380 - protein, 10 aa, Artificial Sequence
  Synthetic
DGGMRRRREN

SEQ ID NO: 381 - protein, 8 aa, Artificial Sequence
  Synthetic
AVIRRPLW

SEQ ID NO: 382 - protein, 10 aa, Artificial Sequence
  Synthetic
DGGMKYTMEN

SEQ ID NO: 383 - protein, 8 aa, Artificial Sequence
  Synthetic
ELVTSRLW

SEQ ID NO: 384 - protein, 10 aa, Artificial Sequence
  Synthetic
DGGVMQLGEN

SEQ ID NO: 385 - protein, 8 aa, Artificial Sequence
  Synthetic
ELGTSRLW

SEQ ID NO: 386 - protein, 8 aa, Artificial Sequence
  Synthetic
FRGWLRWW

SEQ ID NO: 387 - protein, 10 aa, Artificial Sequence
  Synthetic
DDGARVLAEN

SEQ ID NO: 388 - protein, 8 aa, Artificial Sequence
  Synthetic
GRLKGIGW

SEQ ID NO: 389 - protein, 10 aa, Artificial Sequence
  Synthetic
DGGRPQWGEN

SEQ ID NO: 390 - protein, 8 aa, Artificial Sequence
  Synthetic
GVWQSFPW

SEQ ID NO: 391 - protein, 10 aa, Artificial Sequence
  Synthetic
DGGLGYLREN

SEQ ID NO: 392 - protein, 8 aa, Artificial Sequence
  Synthetic
HLVSLAPW

SEQ ID NO: 393 - protein, 10 aa, Artificial Sequence
  Synthetic
DGGGMHQGKN

SEQ ID NO: 394 - protein, 8 aa, Artificial Sequence
  Synthetic
HIFIDWGW

SEQ ID NO: 395 - protein, 10 aa, Artificial Sequence
  Synthetic
DGGVMTMGEN

SEQ ID NO: 396 - protein, 8 aa, Artificial Sequence
  Synthetic
PVMRGVTW

SEQ ID NO: 397 - protein, 10 aa, Artificial Sequence
  Synthetic
DGGRSWVWEN

SEQ ID NO: 398 - protein, 8 aa, Artificial Sequence
  Synthetic
QLVTVGPW

SEQ ID NO: 399 - protein, 10 aa, Artificial Sequence
  Synthetic
DGGVMHRTEN

SEQ ID NO: 400 - protein, 8 aa, Artificial Sequence
  Synthetic
QLVVQMGW

SEQ ID NO: 401 - protein, 10 aa, Artificial Sequence
  Synthetic
DGGWMTVGEN

SEQ ID NO: 402 - protein, 8 aa, Artificial Sequence
  Synthetic
VAIRRSVW

SEQ ID NO: 403 - protein, 10 aa, Artificial Sequence
  Synthetic
DGGERAHSEN

SEQ ID NO: 404 - protein, 8 aa, Artificial Sequence
  Synthetic
WVMRRPLW

SEQ ID NO: 405 - protein, 10 aa, Artificial Sequence
  Synthetic
DGGSMGWREN

SEQ ID NO: 406 - protein, 8 aa, Artificial Sequence
  Synthetic
WRSMVVWW

SEQ ID NO: 407 - protein, 10 aa, Artificial Sequence
  Synthetic
DGGKHTLGEN

SEQ ID NO: 408 - protein, 8 aa, Artificial Sequence
  Synthetic
ELRTDGLW

SEQ ID NO: 409 - protein, 10 aa, Artificial Sequence
  Synthetic
DGGVMRRSEN

SEQ ID NO: 410 - protein, 13 aa, Artificial Sequence
  Synthetic
ACFPIMTLHCGGG

SEQ ID NO: 411 - DNA, 4779 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctaagtgacgatatcctgacctaaggtacctaagtgacgatatcctgacctaactgcagggatcaattgccctacatctgccagttcgggatcgtggcggccgcaggtgcgccggtgccgtatccggatccgctggaaccgcgtgccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 412 - DNA, 10975 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 413 - DNA, 4649 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttctgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 414 - DNA, 10972 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacgtgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 415 - DNA, 10972 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccaggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 416 - DNA, 10969 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagtgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 417 - DNA, 10975 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtcagcctgaaggggaccaaggtgcacatgaaaagctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 418 - DNA, 10927 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 419 - DNA, 4641 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttctgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccgcagttcgaaaaataactcgagataaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 420 - DNA, 11011 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccccagttcgagaagtgactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 421 - DNA, 4101 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctaagtgacgatatcctgacctaaggtacctaagtgacgatatcctgacctaactgcagggatcaattgccctacatctgccagttcgggatcgtggcggccgcaggtgcgccggtgccgtatccggatccgctggaaccgcgtgccgcacaggctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 422 - DNA, 77 bp, Artificial Sequence
  Synthetic
tgggcctgaacgacatggccgccgagggcacctgggtggatatgactggcgcgcgtatcgcctacaagaactgggag

SEQ ID NO: 423 - DNA, 59 bp, Artificial Sequence
  Synthetic
gttgcgccgccatcgggttgmnnmnnmnnmnnmnnctcccagttcttgtaggcgatacg

SEQ ID NO: 424 - DNA, 44 bp, Artificial Sequence
  Synthetic
caacccgatggcggcgcaaccgagaactgcgccgtcctgtctgg

SEQ ID NO: 425 - DNA, 73 bp, Artificial Sequence
  Synthetic
tgtagggcaattgatccctgcagcgcttgtcgaaccacttgccmnnmnnmnngccagacaggacggcgcagtt

SEQ ID NO: 426 - DNA, 31 bp, Artificial Sequence
  Synthetic
gccgagatctggctgggcctgaacgacatgg

SEQ ID NO: 427 - protein, 160 aa, Artificial Sequence
  Synthetic
MELWGALLCLFSLQVTAKAKKKKDVSKMEELKQIDLLKEQQALQTVCLKGTKIHKCFLAFTQKTFHEASEDCISQGGTLSTPQGDENDALYRSVGNEAIWLGNDMAAEGWVDMTGSIYKNWETEITQPDGGKENCAALSANGKWFDKCRDELPYVCQFIV

SEQ ID NO: 428 - protein, 8 aa, Artificial Sequence
  Synthetic
GWLEGSGW

SEQ ID NO: 429 - protein, 8 aa, Artificial Sequence
  synthetic
GYMTGVGW

SEQ ID NO: 430 - protein, 8 aa, Artificial Sequence
  Synthetic
GWMEGVGW

SEQ ID NO: 431 - protein, 8 aa, Artificial Sequence
  synthetic
GWMDGSGW

SEQ ID NO: 432 - protein, 8 aa, Artificial Sequence
  synthetic
GYLAGTGW

SEQ ID NO: 433 - protein, 8 aa, Artificial Sequence
  synthetic
GWLAGSGW

SEQ ID NO: 434 - protein, 8 aa, Artificial Sequence
  Sythetic
GWVAGVGW

SEQ ID NO: 435 - protein, 8 aa, Artificial Sequence
  Synthetic
GWIEGAGW

SEQ ID NO: 436 - protein, 10 aa, Artificial Sequence
  Synthetic
DGGVQWRWEN

SEQ ID NO: 437 - protein, 10 aa, Artificial Sequence
  Synthetic
DGGRSWKWEN

SEQ ID NO: 438 - protein, 10 aa, Artificial Sequence
  Synthetic
DGGPPWRWEN

SEQ ID NO: 439 - protein, 10 aa, Artificial Sequence
  Synthetic
DGGFPARWEN

SEQ ID NO: 440 - protein, 10 aa, Artificial Sequence
  Synthetic
DGGRLWRWEN

SEQ ID NO: 441 - protein, 10 aa, Artificial Sequence
  Synthetic
DGGPGLRWEN

SEQ ID NO: 442 - protein, 10 aa, Artificial Sequence
  Synthetic
DGGRVLAWEN

SEQ ID NO: 443 - protein, 10 aa, Artificial Sequence
  Synthetic
DGGGGWPWEN

SEQ ID NO: 444 - protein, 10 aa, Artificial Sequence
  Synthetic
DGGGGWRWEN

SEQ ID NO: 445 - protein, 10 aa, Artificial Sequence
  Synthetic
DGGWRSRWEN

SEQ ID NO: 446 - protein, 10 aa, Artificial Sequence
  Synthetic
DGGAERAWEN

SEQ ID NO: 447 - DNA, 61 bp, Artificial Sequence
  Synthetic
gagcgtgggcaacgaggccgagatctggctgggcctcaacggttggctggaaggcgtgggt

SEQ ID NO: 448 - DNA, 70 bp, Artificial Sequence
  Synthetic
ccagttcttgtaggcgatacgcgcgccagtcatatccacccaacccacgccttccagccaaccgttgagg

SEQ ID NO: 449 - DNA, 66 bp, Artificial Sequence
  Synthetic
atcgcctacaagaactggnnknnknnknnknnknnkcaacccgatggcggttggccgttcagcaac

SEQ ID NO: 450 - DNA, 72 bp, Artificial Sequence
  Synthetic
cgcttgtcgaaccacttgccgttggcggcgccagacaggacggcgcagttctcgttgctgaacggccaaccg

SEQ ID NO: 451 - protein, 9 aa, Artificial Sequence
  Synthetic
NWTQRHSGQ

SEQ ID NO: 452 - protein, 9 aa, Artificial Sequence
  Synthetic
NWARHINEQ

SEQ ID NO: 453 - protein, 9 aa, Artificial Sequence
  Synthetic
NWYSWPKLQ

SEQ ID NO: 454 - protein, 9 aa, Artificial Sequence
  Synthetic
NWSKVRLEQ

SEQ ID NO: 455 - protein, 9 aa, Artificial Sequence
  Synthetic
NWVAKDHEQ

SEQ ID NO: 456 - protein, 9 aa, Artificial Sequence
  synthetic
NWNSNVVLQ

SEQ ID NO: 457 - protein, 9 aa, Artificial Sequence
  Synthetic
NWGWSARVQ

SEQ ID NO: 458 - protein, 9 aa, Artificial Sequence
  synthetic
NWGWMDSKQ

SEQ ID NO: 459 - protein, 9 aa, Artificial Sequence
  Synthetic
NWWFPTLSQ

SEQ ID NO: 460 - protein, 9 aa, Artificial Sequence
  Synthetic
NWEHPEPWQ

SEQ ID NO: 461 - protein, 9 aa, Artificial Sequence
  Synthetic
NWEPPEPLQ

SEQ ID NO: 462 - protein, 9 aa, Artificial Sequence
  Synthetic
NWHPQGDRQ

SEQ ID NO: 463 - protein, 9 aa, Artificial Sequence
  Synthetic
NWSTAQNGQ

SEQ ID NO: 464 - protein, 9 aa, Artificial Sequence
  Synthetic
NWLDVTKTQ

SEQ ID NO: 465 - protein, 9 aa, Artificial Sequence
  Synthetic
NWAISDERQ

SEQ ID NO: 466 - protein, 9 aa, Artificial Sequence
  Synthetic
NWAEVPFFQ

SEQ ID NO: 467 - protein, 9 aa, Artificial Sequence
  Synthetic
NWWSYWDTQ

SEQ ID NO: 468 - protein, 9 aa, Artificial Sequence
  Synthetic
NWAAVTMEQ

SEQ ID NO: 469 - protein, 9 aa, Artificial Sequence
  Synthetic
NWRVPSLRQ

SEQ ID NO: 470 - protein, 9 aa, Artificial Sequence
  Synthetic
NWSLSWHPQ

SEQ ID NO: 471 - protein, 9 aa, Artificial Sequence
  Synthetic
NWIWSRIEQ

SEQ ID NO: 472 - protein, 9 aa, Artificial Sequence
  Synthetic
NWAAFPVEQ

SEQ ID NO: 473 - protein, 9 aa, Artificial Sequence
  Synthetic
NWGSTGEKQ

SEQ ID NO: 474 - protein, 9 aa, Artificial Sequence
  Synthetic
NWGEVIAPQ

SEQ ID NO: 475 - protein, 9 aa, Artificial Sequence
  Synthetic
NWFAEFFLQ

SEQ ID NO: 476 - protein, 9 aa, Artificial Sequence
  Synthetic
NWGRRRNLQ

SEQ ID NO: 477 - protein, 9 aa, Artificial Sequence
  Synthetic
NWGSYGPFQ

SEQ ID NO: 478 - protein, 9 aa, Artificial Sequence
  Synthetic
NWGTHISSQ

SEQ ID NO: 479 - protein, 9 aa, Artificial Sequence
  Synthetic
NWGTGVMGQ

SEQ ID NO: 480 - protein, 9 aa, Artificial Sequence
  synthetic
NWGGSISAQ

SEQ ID NO: 481 - protein, 9 aa, Artificial Sequence
  Synthetic
NWGGEVLLQ

SEQ ID NO: 482 - protein, 9 aa, Artificial Sequence
  Synthetic
NWSEDRPGQ

SEQ ID NO: 483 - protein, 9 aa, Artificial Sequence
  Synthetic
NWVYRPGMQ

SEQ ID NO: 484 - protein, 9 aa, Artificial Sequence
  Synthetic
NWVNHGVGQ

SEQ ID NO: 485 - protein, 9 aa, Artificial Sequence
  Synthetic
NWQPGLWRQ

SEQ ID NO: 486 - protein, 9 aa, Artificial Sequence
  Synthetic
NWQVHARSQ

SEQ ID NO: 487 - protein, 9 aa, Artificial Sequence
  Synthetic
NWAMHYYWQ

SEQ ID NO: 488 - protein, 9 aa, Artificial Sequence
  Synthetic
NWDAPVSGQ

SEQ ID NO: 489 - protein, 9 aa, Artificial Sequence
  Synthetic
NWFIPADRQ

SEQ ID NO: 490 - protein, 9 aa, Artificial Sequence
  synthetic
NWYVRSEGQ

SEQ ID NO: 491 - protein, 10 aa, Artificial Sequence
  Synthetic
NWEHPEPWHQ

SEQ ID NO: 492 - DNA, 61 bp, Artificial Sequence
  Synthetic
gagcgtgggcaacgaggccgagatctggctgggcctcaacggttggctggaaggctctggt

SEQ ID NO: 493 - DNA, 70 bp, Artificial Sequence
  Synthetic
ccagttcttgtaggcgatacgcgcgccagtcatatccacccaaccagagccttccagccaaccgttgagg

SEQ ID NO: 494 - DNA, 66 bp, Artificial Sequence
  Synthetic
atcgcctacaagaactggnnknnknnknnknnknnkcaacccgatggcggtgttcagtggaggtgg

SEQ ID NO: 495 - DNA, 72 bp, Artificial Sequence
  Synthetic
cgcttgtcgaaccacttgccgttggcggcgccagacaggacggcgcagttctcccacctccactgaacaccg

SEQ ID NO: 496 - protein, 9 aa, Artificial Sequence
  Synthetic
NWGDQRLAQ

SEQ ID NO: 497 - protein, 9 aa, Artificial Sequence
  Synthetic
NWADERRNQ

SEQ ID NO: 498 - protein, 9 aa, Artificial Sequence
  Synthetic
NWADKRWLQ

SEQ ID NO: 499 - protein, 9 aa, Artificial Sequence
  Synthetic
NWADKRWLQ

SEQ ID NO: 500 - protein, 9 aa, Artificial Sequence
  Synthetic
NWLDPRMGQ

SEQ ID NO: 501 - protein, 9 aa, Artificial Sequence
  Synthetic
NWYSDYLNQ

SEQ ID NO: 502 - protein, 9 aa, Artificial Sequence
  Synthetic
NWHYQKYIQ

SEQ ID NO: 503 - protein, 9 aa, Artificial Sequence
  Synthetic
NWALDRYNQ

SEQ ID NO: 504 - protein, 9 aa, Artificial Sequence
  Synthetic
NWGRPELAQ

SEQ ID NO: 505 - protein, 9 aa, Artificial Sequence
  Synthetic
NWANPSFMQ

SEQ ID NO: 506 - protein, 9 aa, Artificial Sequence
  Synthetic
NWADERFLQ

SEQ ID NO: 507 - protein, 9 aa, Artificial Sequence
  Synthetic
NWGRRELAQ

SEQ ID NO: 508 - DNA, 60 bp, Artificial Sequence
  Synthetic
ttcgcaattcctttagtggtacctttctattctcactctgctagcatggccgccctccag

SEQ ID NO: 509 - DNA, 20 bp, Artificial Sequence
  Synthetic
agtctatgcggcacgcggtt

SEQ ID NO: 510 - protein, 36 aa, Artificial Sequence
  Synthetic
GGTGGAGCTAGCGTTGTGAACACAAAGATGTTTGAG

SEQ ID NO: 511 - protein, 39 aa, Artificial Sequence
  Synthetic
GTGCACTGCGGCCGCCTTCAGGCAGACCGTCTGGAGGGC

SEQ ID NO: 512 - DNA, 64 bp, Artificial Sequence
  Synthetic
nnknnknnknnknnknnknnknnknnknnknnknnknnknnknnkggtggcggttcggctgaag

SEQ ID NO: 513 - DNA, 20 bp, Artificial Sequence
  Synthetic
agtctatgcggcacgcggtt

SEQ ID NO: 514 - DNA, 27 bp, Artificial Sequence
  Synthetic
aacctggtacctttctattctcactcc

SEQ ID NO: 515 - protein, 15 aa, Artificial Sequence
  Synthetic
FYPSVCLTSCASIQR

SEQ ID NO: 516 - protein, 15 aa, Artificial Sequence
  Synthetic
MHMTPPYLCRWGCAT

SEQ ID NO: 517 - protein, 15 aa, Artificial Sequence
  Synthetic
VVMNGPFLCRTPCLV

SEQ ID NO: 518 - protein, 15 aa, Artificial Sequence
  Synthetic
QGPTIMGPYLCTYGC

SEQ ID NO: 519 - protein, 15 aa, Artificial Sequence
  Synthetic
GGCLPYLTCRMGSVT

SEQ ID NO: 520 - protein, 15 aa, Artificial Sequence
  Synthetic
QMNCRPILTCKHRTL

SEQ ID NO: 521 - protein, 15 aa, Artificial Sequence
  Synthetic
QEGWTFSCMPYLTCR

SEQ ID NO: 522 - protein, 15 aa, Artificial Sequence
  Synthetic
WTASSKFCSRPFLTC

SEQ ID NO: 523 - protein, 17 aa, Artificial Sequence
  Synthetic
TKIDDNALVITQKARWR

SEQ ID NO: 524 - protein, 15 aa, Artificial Sequence
  Synthetic
MHMTPPYLCRWGCAT

SEQ ID NO: 525 - protein, 14 aa, Artificial Sequence
  Synthetic
HMTPPYLCRWGCAT

SEQ ID NO: 526 - protein, 13 aa, Artificial Sequence
  Synthetic
MTPPYLCRWGCAT

SEQ ID NO: 527 - protein, 12 aa, Artificial Sequence
  Synthetic
TPPYLCRWGCAT

SEQ ID NO: 528 - protein, 11 aa, Artificial Sequence
  Synthetic
PPYLCRWGCAT

SEQ ID NO: 529 - protein, 10 aa, Artificial Sequence
  Synthetic
PYLCRWGCAT

SEQ ID NO: 530 - protein, 9 aa, Artificial Sequence
  Synthetic
YLCRWGCAT

SEQ ID NO: 531 - protein, 15 aa, Artificial Sequence
  Synthetic
MHMTAPYLCRWGCAT

SEQ ID NO: 532 - protein, 15 aa, Artificial Sequence
  synthetic
MHMTPAYLCRWGCAT

SEQ ID NO: 533 - protein, 15 aa, Artificial Sequence
  Synthetic
MHMTPPALCRWGCAT

SEQ ID NO: 534 - protein, 15 aa, Artificial Sequence
  Synthetic
MHMTPPYACRWGCAT

SEQ ID NO: 535 - protein, 15 aa, Artificial Sequence
  Synthetic
MHMTPPYLCAWGCAT

SEQ ID NO: 536 - protein, 15 aa, Artificial Sequence
  Synthetic
MHMTPPYLCRAGCAT

SEQ ID NO: 537 - protein, 15 aa, Artificial Sequence
  Synthetic
MHMTPPYLCRWACAT

SEQ ID NO: 538 - protein, 15 aa, Artificial Sequence
  Synthetic
MHMTPPYLCRWGCAA

SEQ ID NO: 539 - protein, 9 aa, Artificial Sequence
  Synthetic
NWYDPVYDQ

SEQ ID NO: 540 - protein, 9 aa, Artificial Sequence
  Synthetic
NWASEVFQQ

SEQ ID NO: 541 - protein, 9 aa, Artificial Sequence
  Synthetic
NWADARWDQ

SEQ ID NO: 542 - protein, 9 aa, Artificial Sequence
  Synthetic
NWADDRWNQ

SEQ ID NO: 543 - protein, 9 aa, Artificial Sequence
  Synthetic
NWAYSKWNQ

SEQ ID NO: 544 - protein, 9 aa, Artificial Sequence
  synthetic
NWANQRWNQ

SEQ ID NO: 545 - protein, 9 aa, Artificial Sequence
  Synthetic
NWGDPRWSQ

SEQ ID NO: 546 - protein, 9 aa, Artificial Sequence
  Synthetic
NWANLRFNQ

SEQ ID NO: 547 - protein, 9 aa, Artificial Sequence
  Synthetic
NWADPTWSQ

SEQ ID NO: 548 - protein, 9 aa, Artificial Sequence
  Synthetic
NWGDSRFMQ

SEQ ID NO: 549 - protein, 9 aa, Artificial Sequence
  Synthetic
NWGNPRWGQ

SEQ ID NO: 550 - protein, 9 aa, Artificial Sequence
  Synthetic
NWGTPRLAQ

SEQ ID NO: 551 - protein, 9 aa, Artificial Sequence
  synthetic
NWAPGVVAQ

SEQ ID NO: 552 - protein, 9 aa, Artificial Sequence
  Synthetic
NWGHGDLWQ

SEQ ID NO: 553 - protein, 9 aa, Artificial Sequence
  Synthetic
NWYNASFFQ

SEQ ID NO: 554 - protein, 9 aa, Artificial Sequence
  Synthetic
NWGDARFGQ

SEQ ID NO: 555 - protein, 9 aa, Artificial Sequence
  Synthetic
NWAEARLWQ

SEQ ID NO: 556 - protein, 9 aa, Artificial Sequence
  Synthetic
NWAEARWWQ

SEQ ID NO: 557 - protein, 9 aa, Artificial Sequence
  Synthetic
NWAVDTFNQ

SEQ ID NO: 558 - protein, 9 aa, Artificial Sequence
  synthetic
NWARDIFNQ

SEQ ID NO: 559 - protein, 9 aa, Artificial Sequence
  Synthetic
NWGGWLADQ

SEQ ID NO: 560 - protein, 9 aa, Artificial Sequence
  Synthetic
NWGDARWAQ

SEQ ID NO: 561 - protein, 9 aa, Artificial Sequence
  Synthetic
NWADERWSQ

SEQ ID NO: 562 - protein, 9 aa, Artificial Sequence
  synthetic
NWADERWSQ

SEQ ID NO: 563 - protein, 15 aa, Artificial Sequence
  synthetic
XXXXXPXLXRXGXXX

SEQ ID NO: 564 - DNA, 10987 bp, Artificial Sequence
  synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagggtttaaactttagcttaccgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgacaagctttccagctagcggtggaggttcggttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccccagttcgagaagtgactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 565 - DNA, 4644 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttcttggagccacccccagttcgagaagggttacccctacgacgtgcccgactacgccgccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtaactcgagataaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 566 - DNA, 11014 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccacctggagccacccccagttcgagaagggttacccctacgacgtgcccgactacgccgccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 567 - DNA, 4588 bp, Artificial Sequence
  synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagtggtacctttctattctcactctgctagcgttgtgaacacaaagatgtttgaggagctcaagagccgtctggacaccctggcccaggaggtggccctgctgaaggagcagcaggccctccagacggtctgcctgaaggcggccgcaggtgcgccggtgccgtatccggatccgctggaaccgcgtgccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 568 - DNA, 11287 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggnccgggcctcggttccagaagggagaggagcccgccaaggcgcgcaagagagcgggctgcctcgcagtccgagccggagagggagcgcgagccgcgccggccccggacggcctccgaaaccatggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagaaatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaaccgggccgtgttgcggccccggtgggtggacatgactggcgcgcgtatcgcctacaagaactgggagactgagatcaccgcgcaacccgatggcggctgggtgttgggggggaagaactgcgcggtcctgtcaggcgcggccaacggcaagtggttcgacaagcgctgcagggatcaattgccctacatctgccagttcgggatcgtgtgactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 569 - protein, 9 aa, Artificial Sequence
  Synthetic
NWADPKWSQ

SEQ ID NO: 570 - protein, 9 aa, Artificial Sequence
  Synthetic
NWFHDRFNQ